(19) United States
(12) Patent Application Publication
(10) Pub. No.: US 2014/0205537 A1
(43) Pub. Date: Jul. 24, 2014

(54) LRRK2 INHIBITORS

(71) Applicants: Pierre-Yves Bounaud, San Diego, CA (US); Vicki Nienaber, La Jolla, CA (US); Ruo W. Steensma, La Jolla, CA (US); John A. Lowe, III, Stonington, CT (US)

(72) Inventors: Pierre-Yves Bounaud, San Diego, CA (US); Vicki Nienaber, La Jolla, CA (US); Ruo W. Steensma, La Jolla, CA (US); John A. Lowe, III, Stonington, CT (US)

(73) Assignee: ZENOBIA THERAPEUTICS, INC., San Diego, CA (US)

(21) Appl. No.: 14/129,099

(22) PCT Filed: Jun. 22, 2012
(86) PCT No.: PCT/US2012/043757

§ 371 Date: Mar. 27, 2014

Related U.S. Application Data
(60) Provisional application No. 61/501,103, filed on Jun. 24, 2011.
(60) Provisional application No. 61/613,847, filed on Mar. 21, 2012.

(51) Int. Cl. C07D 403/06; C07D 417/06; C07D 401/14; C07D 413/06; C07D 405/14; C07D 471/04; C07D 401/06

(52) U.S. Cl. CPC C07D 403/06; C07D 471/04; C07D 417/06; C07D 401/06; C07D 413/06; C07D 405/14; C07D 401/14
USPC 424/1.65; 548/364.7; 514/406; 548/312.1; 514/397; 546/113; 514/300; 548/159; 514/367; 546/174; 514/314; 548/217; 514/375; 544/139; 514/235.2; 435/184

(57) ABSTRACT

Provided herein are compounds that inhibit or partially inhibit the activity of leucine rich repeat kinases. Also provided herein are methods of treatment of CNS disorders comprising administration of inhibitors of leucine rich repeat kinases.

### CROSS-REFERENCE

This application claims the benefit of U.S. Provisional Application No. 61/501,103, filed Jun. 24, 2011 and U.S. Provisional Application No. 61/613,847, filed Mar. 21, 2012, which are incorporated herein by reference in their entireties.

### BACKGROUND OF THE INVENTION

Neurodegenerative disorders in an ageing population have profound socio-economic effects and represent a large health care burden.

### SUMMARY OF THE INVENTION

Targeted therapies that alter the course of disease have been very successful in a number of disease areas (e.g. oncology, anti-viral, anti-infective, anti-inflammatory). However, in the central nervous system (CNS) disease area, targeted therapies have not been very successful to date and generally do not alter the course of the disease. Described herein is a targeted, disease modifying approach for treatment of CNS disorders.

In one aspect provided herein are compounds, or salt thereof, of formula (I-Z-1):

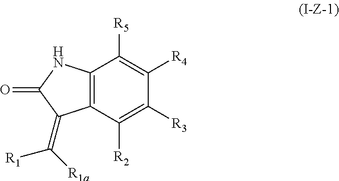

wherein
R₁ is pyrazole, imidazole, triazole, triazolone, indole, benzimidazole, azabenzimidazole, azaindole, benzothiazole or benzoxazole, where R₁ is optionally substituted with one, two, three, four, or five R₆;
   each R₆ is independently hydroxy, halo, alkyl, carbocyclyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₁ₐ is hydrogen, optionally substituted alkyl, optionally substituted heteroalkyl, optionally substituted cycloalkyl or optionally substituted heteroalicyclyl;
each of R₂, R₃, and R₄ is independently hydrogen, hydroxy, halo, optionally substituted alkyl, optionally substituted cycloalkyl, optionally substituted cycloalkyloxy, optionally substituted alkoxy, optionally substituted aryl, optionally substituted heteroaryl, or optionally substituted heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R₅ is hydrogen, halo, alkyl, haloalkyl;
   R′ and R″ taken together with the nitrogen to which they are attached form a ring structure that optionally includes an additional heteroatom selected from N or O and is optionally substituted;
   provided that at least one of R₂, R₃ and R₄ is not hydrogen; and
   further provided that when R₁ is indol-3-yl, R₃ is not Cl, and when R₁ is imidazo-2-yl, R₃ is not Cl;
where alkyl, alkoxy, cycloalkyloxy, heteroalkyl, carbocyclyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, cycloalkyl and heteroalicyclyl.

In some embodiments of formula (I-Z-1), R₁ is an imidazole, wherein the imidazole has a structure of:

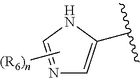

(imidazol-5-yl) or

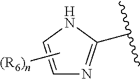

(imidazol-2-yl); and n is 0, 1, 2, or 3, where R₁, R₁ₐ, R₂, R₃, R₄, R₅ and R₆ are as described below and herein.

In some embodiments of formula (I-Z-1), R₁ is an imidazole, wherein the imidazole has a structure of:

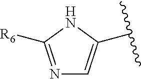

(imidazol-5-yl) or

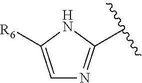

(imidazol-2-yl); and
where R₁, R₁ₐ, R₂, R₃, R₄, R₅ and R₆ are as described below and herein. In some embodiments of formula (I-Z-1) described above, R₁ is imidazol-5-yl. In some embodiments of formula (I-Z-1) described above, R₁ is imidazol-2-yl.

In one aspect, provided herein is a compound, or salt thereof, of formula (I-Z-2):

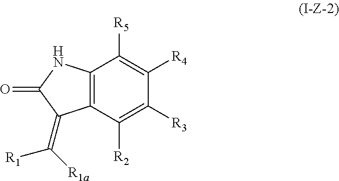

wherein
R₁ is pyrazole, imidazol-5-yl, triazolyl, triazolonyl, indoly-2-yl, indol-4-yl, indol-5-yl, indole-6-yl, indol-7-yl, benzimidazolyl, azabenzimidazolyl, azaindolyl, benzothiazolyl or benzoxazolyl, where R₁ is optionally substituted with one, two, three, four, or five R₆;
   each R₆ is independently hydroxy, halo, optionally substituted alkyl, optionally substituted alkoxy, optionally substituted cycloalkyl, optionally substituted aryl, optionally substituted heteroaryl, optionally substituted heteroalkyl optionally substituted heteroalicyclyl, optionally substituted alkylcycloalkyl, optionally substituted alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₁ₐ is hydrogen, optionally substituted alkyl, optionally substituted heteroalkyl, optionally substituted cycloalkyl or optionally substituted heteroalicyclyl;
each of R₂ and R₄ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₃ is hydroxy, halo, optionally substituted alkyl, optionally substituted cycloalkyl, optionally substituted alkoxy, optionally substituted cycloalkyloxy, optionally substituted aryl, optionally substituted heteroaryl, or optionally substituted heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₅ is hydrogen, halo, haloalkyl or alkyl; and
   each of R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted.

In an embodiment of compounds of formula (I-Z-1) or formula (I-Z-2), alkyl, alkoxy, heteroalkyl, cycloalkyl, cycloalkyloxy, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, cycloalkyl and heteroalicyclyl.

In some embodiments, compounds of formula (I-Z-2), or salt thereof, have the structure of formula (I-Z-2-a), formula (I-Z-2-b), formula (I-Z-2-c) or formula (I-Z-2-d):

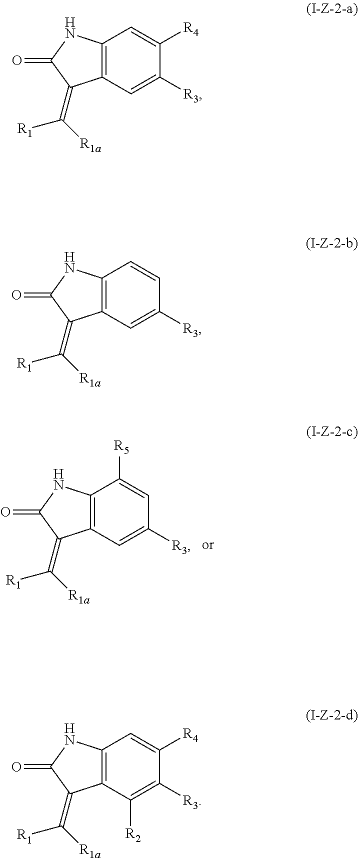

In a specific embodiment, compounds of formula (I-Z-2), or salt thereof, have the structure of formula (I-Z-2-a). In a specific embodiment, compounds of formula (I-Z-2), or salt thereof, have the structure of formula (I-Z-2-b). In a specific embodiment, compounds of formula (I-Z-2), or salt thereof, have the structure of formula (I-Z-2-c). In a specific embodiment, compounds of formula (I-Z-2), or salt thereof, have the structure of formula (I-Z-2-d).

For any preceding compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is hydrogen. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is optionally substituted alkyl. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is C₁-C₃ alkyl. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is methyl.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is an optionally substituted alkyl or optionally substituted heteroalkyl. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is an optionally substituted cycloalkyl or optionally substituted hetroalicyclyl. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is an optionally substituted heteroalicyclyl. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is an optionally substituted cycloalkyl.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ is pyrazolyl, imidazol-5-yl, triazolyl, triazolonyl, indoly-2-yl, indol-4-yl, indol-5-yl, indole-6-yl, indol-7-yl, benzimidazolyl, or azaindolyl where R₁ is optionally substituted with one or two R₆.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ is pyrazolyl, imidazol-5-yl, triazolyl, or triazolonyl, where R₁ is optionally substituted with one or two R₆. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ is pyrazolyl, or imidazol-5-yl, where R₁ is optionally substituted with one or two R₆. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ is indoly-2-yl, indol-4-yl, indol-5-yl, indole-6-yl, indol-7-yl, benzimidazolyl, or azaindolyl where R₁ is optionally substituted with one or two R₆.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ is imidazol-5-yl and has a structure of:

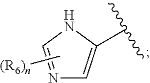

and
n is 0, 1, 2, or 3.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ is imidazol-5-yl, optionally substituted with one R₆.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ and R₁ are as described above, and R₆ is independently hydroxy, halo, haloalkyl, haloalkoxy, alkyl, cycloalkyl, alkoxy, aryl, heteroaryl, heteroalkyl, optionally substituted heteroalicyclyl, alkylcycloalkyl, alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″, where cycloalkyl, aryl, heteroaryl and heteroalicyclyl are optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, cycloalkyl and heteroalicyclyl, and alkyl and alkoxy are optionally substituted with one or two groups selected from oxo, hydroxy, amino, cyano, aminoalkyl, aminodialkyl, heteroalkyl, S(O)₂R, NR′C(O)R″, C(O)NR′R″, cycloalkyl and heteroalicyclyl; and where R, R′ and R″ are independently hydrogen, alkyl, haloalkyl, cycloalkyl, aryl, heteroaryl, or heteroalkyl.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ and R₁ are as described above, and R₆ is independently hydroxy, halo, optionally substituted C₁-C₆alkyl, optionally substituted C₂-C₆ heteroalkyl, optionally substituted C₃-C₇cycloalkyl, optionally substituted C₁-C₆alkoxy, optionally substituted aryl, optionally substituted heteroaryl, optionally substituted C₃-C₇heteroalicyclyl, optionally substituted C₁-C₃alkyl-C₃-C₇cycloalkyl, optionally substituted C₁-C₃alkyl-C₃-C₇heteroalicyclyl, or NR′R″, where alkyl, alkoxy, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, SO₂(C₁-C₃alkyl), NR′C(O)R″, C(O)NR′R″, C₃-C₆cycloalkyl, C₃-C₆heteroalicyclyl and C₂-C₆heteroalkyl; and where R′ and R″ are independently hydrogen, C₁-C₆alkyl, haloC₁-C₆alkyl, C₃-C₇cycloalkyl, aryl, heteroaryl, or C₁-C₆heteroalkyl.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ and R₁ are as described above, and R₆ is independently hydroxy, halo, optionally substituted C₁-C₆alkyl, optionally substituted C₂-C₆ heteroalkyl, optionally substituted C₃-C₇cycloalkyl, optionally substituted C₁-C₆alkoxy, optionally substituted aryl, optionally substituted heteroaryl, optionally substituted C₃-C₇heteroalicyclyl, optionally substituted C₁-C₃alkyl-C₃-C₇cycloalkyl, optionally substituted C₁-C₃alkyl-C₃-C₇heteroalicyclyl, or NR′R″, where alkyl, alkoxy, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, SO₂(C₁-C₃alkyl), C₃-C₆cycloalkyl, C₃-C₆heteroalicyclyl and C₂-C₆heteroalkyl; and where R′ and R″ are independently hydrogen, C₁-C₆alkyl, haloC₁-C₆alkyl, C₃-C₇cycloalkyl, aryl, heteroaryl, or C₁-C₆heteroalkyl.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ and R₁ are as described above, and R₆ is independently hydroxy, halo, haloC₁-C₆alkyl, haloC₁-C₆alkoxy, C₁-C₆alkyl, optionally substituted C₂-C₆ heteroalkyl, optionally substituted C₃-C₇cycloalkyl, C₁-C₆alkoxy, optionally substituted aryl, optionally substituted heteroaryl, optionally substituted C₃-C₇heteroalicyclyl, optionally substituted C₁-C₃alkyl-C₃-C₇cycloalkyl, or optionally substituted C₁-C₃alkyl-C₃-C₇heteroalicyclyl, where heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, SO₂(C₁-C₃alkyl), C₃-C₆cycloalkyl, and C₃-C₇heteroaliyclyl, and alkyl and alkoxy are optionally substituted with one or two groups selected from oxo, hydroxy, amino, cyano, amino(C₁-C₆alkyl), amino(C₁-C₆alkyl)₂, SO₂ (C₁-C₃alkyl), C₃-C₆cycloalkyl, and C₃-C₇heteroaliyclyl.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ and R₁ are as described above, and R₆ is independently hydroxy, F, Cl, Br, I, CH₂F, CHF₂, CF₃, CH₂CH₂F, CH₂CHF₂, CH₂CF₃, methyl, ethyl, propyl, butyl, pentyl, hexyl, iso-propyl, cyclopropyl, cyclobutyl, cyclopentyl, cyclohexyl, methoxy, ethoxy, propoxy, iso-propoxy, phenyl, pyridyl, OCH₂F, OCHF₂, OCF₃, OC(═O)Me, CO₂Me, CO₂Et, CO₂H, NHC(O)Me, C(O)NMe₂, C(O)NH₂, C(O)NHMe, SO₂Me, SO₂Et, or SO₂NMe₂.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ and R₁ are as described above, and R₆ is independently optionally substituted C₁-C₆alkyl, optionally substituted C₂-C₆heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂ (C₁-C₃alkyl), C₃-C₆cycloalkyl, and C₃-C₇heteroaliyclyl; and R′ and R″ are independently selected from hydrogen and C₁-C₆ alkyl.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ and R₁ are as described above, and R₆ is methyl, ethyl, propyl, isopropyl, butyl, isobutyl, tert-butyl, pentyl, neo-pentyl, or isobutyl; or R₆ is tetrahydropyranyl, piperidinyl, piperazinyl, morpholinyl, pyrrolidinyl, pyrrolyl, phenyl, pyridinyl, or pyrimidinyl, where tetrahydropyranyl, piperidinyl, piperazinyl, pyrrolidinyl, pyrrolyl, phenyl, pyridinyl, or pyrimidinyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂ (C₁-C₃alkyl), C₃-C₆cycloalkyl, and C₃-C₇heteroaliyclyl. In some related embodiments, R₁ₐ and R₁ are as described above, and R₆ is an optionally substituted C₃-C₇heteroalicyclyl selected from tetrahydropyranyl, piperidinyl, piperazinyl, morpholinyl, and pyrrolidinyl, which are optionally substituted with one or two groups selected from oxo, hydroxy, F, Cl, Br, I, CH₂F, CHF₂, CF₃, CH₂CH₂F, CH₂CHF₂, CH₂CF₃, methyl, ethyl, propyl, butyl, pentyl, hexyl, iso-propyl, cyclopropyl, cyclobutyl, methoxy, ethoxy, propoxy, iso-propoxy, OCF₃, OCHF₂, OCH₂CH₂NH₂, NHCH₂CH₂OH, OCH₂CH₂N(CH₃)₂, and NHCH₂CH₂OCH₃.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁ and R₆ are as described above, and
R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
   each of R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁ and R₆ are as described above, and R₂ is hydrogen, hydroxy, F, Cl, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, methoxy, ethoxy, iso-propoxy, OCF₃, C(O)NMe₂, or SO₂Me.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁ and R₆ are as described above, and R₂ is hydrogen.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is halo, haloalkyl, haloalkoxy, alkyl, or alkoxy. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is haloor alkoxy. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is haloor optionally substituted alkoxy. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is haloor optionally substituted alkoxy or optionally substituted cycloalkyloxy.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and
R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
   each of R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is hydroxy, CH₂F, CHF₂, CF₃, CH₂CH₂F, CH₂CHF₂, CH₂CF₃, methyl, ethyl, methoxy, ethoxy, propoxy, iso-propyl, iso-propoxy, cyclopropyl, cyclobutyl, cyclopentyl, OCF₃, C(O)NMe₂, or SO₂Me. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is methoxy, ethoxy, propoxy, or iso-propoxy. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is methoxy, ethoxy, propoxy, iso-propoxy, butoxy, or isobutoxy. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is F or Cl. For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆ and R₂ are as described above, and R₃ is cyclopropyloxy or cyclobutyloxy.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆, R₂ and R₃ are as described above, and R₄ is hydrogen, hydroxy, halo, haloC₁-C₆alkyl, C₁-C₆alkyl or C₁-C₆alkoxy.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆, R₂ and R₃ are as described above, and R₄ is hydrogen, hydroxy, F, Cl, Br, I, CH₂F, CHF₂, CF₃, CH₂CH₂F, CH₂CHF₂, CH₂CF₃, methyl, methoxy, ethyl, ethoxy, propyl, propoxy, iso-propyl or isopropoxy.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆, R₂ and R₃ are as described above, and R₄ is hydrogen or fluoro.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆, R₂ and R₃ are as described above, and R₄ is C₁-C₃alkoxy.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆, R₂, R₃ and R₄ are as described above, and R₅ is hydrogen, halo, alkyl or haloalkyl.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆, R₂, R₃ and R₄ are as described above, and R₅ is hydrogen or halo.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₁, R₆, R₂, R₃ and R₄ are as described above, and R₅ is hydrogen or fluoro.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₂, R₄ and R₅ are as described above, and
R₁ is imidazol-5-yl optionally substituted with one or two R₆;
R₆ is independently optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl; and
R₃ is hydroxy, halo, haloalkyl, haloalkoxy, alkyl, or alkoxy.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ, R₂, R₃ and R₅ are as described above, and
R₁ is imidazol-5-yl optionally substituted with one or two R₆;
R₆ is independently optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl; and
R₄ is hydrogen, halo, haloalkyl, haloalkoxy, alkyl, or alkoxy.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is as described above, and
R₁ is imidazol-5-yl optionally substituted with one or two R₆;
R₆ is independently optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl; and
R₃ is hydroxy, halo, haloalkyl, haloalkoxy, alkyl, or alkoxy;
R₄ is hydrogen, hydroxy, halo, haloalkyl, haloalkoxy, alkyl, or alkoxy; and
R₂ and R₅ are hydrogen.

For any compound of formula (I-Z-1) or formula (I-Z-2), formula (I-Z-2-a), formula (I-Z-2-b), formula (I-Z-2-c) or formula (I-Z-2-d), or salt thereof, in one embodiment, R₁ₐ is as described above, and
R₁ is imidazol-5-yl optionally substituted with one or two R₆;
R₆ is independently optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl; and
R₃ is hydroxy, halo, haloalkyl, haloalkoxy, alkyl, or alkoxy;
R₅ is hydrogen, or halo; and
R₂ and R₄ are hydrogen.

In another aspect, provided herein is a compound, or salt thereof, of Formula (I-Z-3-a):

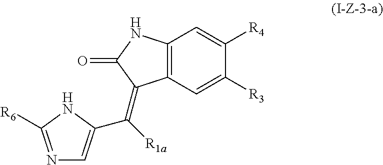

wherein
R₁ₐ is hydrogen, optionally substituted C₁-C₆ alkyl, optionally substituted C₃-C₇ cycloalkyl or optionally substituted C₃-C₇ heteroalicyclyl;
R₆ is optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, or optionally substituted heteroaryl;
R₃ is halo, alkoxy or haloalkoxy;
R₄ is hydrogen or halo;
where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl.

In another aspect, provided herein is a compound, or salt thereof, of Formula (I-Z-3-b):

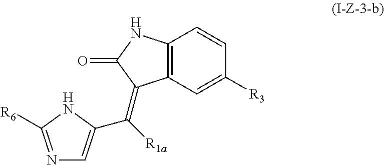

wherein
R₁ₐ is hydrogen, optionally substituted C₁-C₆ alkyl, optionally substituted C₃-C₇ cycloalkyl or optionally substituted C₃-C₇ heteroalicyclyl;
R₆ is optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, or optionally substituted heteroaryl;
R₃ is halo, alkoxy or haloalkoxy;
where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl.

In another aspect, provided herein is a compound, or salt thereof, of Formula (I-Z-3-c):

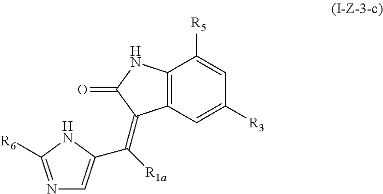

wherein
R₁ₐ is hydrogen, optionally substituted C₁-C₆ alkyl, optionally substituted C₃-C₇ cycloalkyl or optionally substituted C₃-C₇ heteroalicyclyl;
R₆ is optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, or optionally substituted heteroaryl;
R₃ is halo, alkoxy or haloalkoxy;
R₅ is hydrogen or halo;
where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl.

In another aspect, provided herein is a compound, or salt thereof, of Formula (I-Z-3-d):

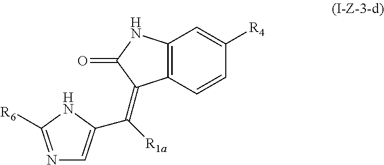

wherein
R₁ₐ is hydrogen, optionally substituted C₁-C₆ alkyl, optionally substituted C₃-C₇ cycloalkyl or optionally substituted C₃-C₇ heteroalicyclyl;
R₆ is optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, or optionally substituted heteroaryl;
R₄ is halo, alkoxy or haloalkoxy;
where alkyl, heteroalkyl, cycloalkyl, aryl, heteroaryl and heteroalicyclyl are optionally substituted with one or two groups selected from oxo, hydroxy, amino, cyano, halo,

C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl.

For any compound of formula (I-Z-3-a), formula (I-Z-3-b), formula (I-Z-3-c) or formula (I-Z-3-d), or salt thereof, in one embodiment, R₁ₐ is H. For any compound of formula (I-Z-3-a), formula (I-Z-3-b), formula (I-Z-3-c) or formula (I-Z-3-d), or salt thereof, in one embodiment, R₁ₐ is methyl. For any compound of formula (I-Z-3-a), formula (I-Z-3-b), formula (I-Z-3-c) or formula (I-Z-3-d), or salt thereof, in one embodiment, R₁ₐ is optionally substituted C₁-C₆ alkyl. For any compound of formula (I-Z-3-a), formula (I-Z-3-b), formula (I-Z-3-c) or formula (I-Z-3-d), or salt thereof, in one embodiment, R₁ₐ is optionally substituted C₃-C₇ cycloalkyl. For any compound of formula (I-Z-3-a), formula (I-Z-3-b), formula (I-Z-3-c) or formula (I-Z-3-d), or salt thereof, in one embodiment, R₁ₐ is optionally substituted C₃-C₇ heteroalicyclyl.

For any compound of formula (I-Z-3-a), formula (I-Z-3-b), formula (I-Z-3-c) or formula (I-Z-3-d), or salt thereof, in one embodiment, R₁ₐ is as described above and R₆ is methyl, ethyl, propyl, isopropyl, butyl, isobutyl, tert-butyl, pentyl, neo-pentyl, or isobutyl. For any compound of formula (I-Z-3-a), formula (I-Z-3-b), formula (I-Z-3-c) or formula (I-Z-3-d), or salt thereof, in one embodiment, R₁ₐ is as described above and R₆ is tetrahydropyranyl, piperidinyl, piperazinyl, morpholinyl, pyrrolidinyl, pyrrolyl, phenyl, pyridinyl, or pyrimidinyl, where tetrahydropyranyl, piperidinyl, piperazinyl, pyrrolidinyl, pyrrolyl, phenyl, pyridinyl, or pyrimidinyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl) and C₃-C₇heteroaliyclyl. In some related embodiments, R₁ₐ and is as described above, and R₆ is an optionally substituted C₃-C₇heteroalicyclyl selected from tetrahydropyranyl, piperidinyl, piperazinyl, morpholinyl, and pyrrolidinyl, which are optionally substituted with one or two groups selected from oxo, hydroxy, F, Cl, Br, I, CH₂F, CHF₂, CF₃, CH₂CH₂F, CH₂CHF₂, CH₂CF₃, methyl, ethyl, propyl, butyl, pentyl, hexyl, iso-propyl, cyclopropyl, cyclobutyl, methoxy, ethoxy, propoxy, iso-propoxy, OCF₃, OCHF₂, OCH₂CH₂NH₂, NHCH₂CH₂OH, OCH₂CH₂N(CH₃)₂, and NHCH₂CH₂OCH₃.

For any compound of formula (I-Z-3-a), R₁ₐ and R₆ are as described above, and in some cases, R₃ is halo, O(C₁-C₆alkyl) or O(C₁-C₆)haloalkyl; or R₃ is F, Cl, methoxy, ethoxy, propoxy, butoxy, isopropoxy, isobutyloxy or OCF₃. For any compound of formula (I-Z-3-a), R₁ₐ, R₆ and R₃ are as described above, and in some cases, R₄ is hydrogen or F or Cl.

For any compound of formula (I-Z-3-b), R₁ₐ and R₆ are as described above, and in some cases, R₃ is halo, O(C₁-C₆alkyl) or O(C₁-C₆)haloalkyl; or R₃ is F, Cl, methoxy, ethoxy, propoxy, butoxy, isopropoxy, isobutyloxy or OCF₃.

For any compound of formula (I-Z-3-c), R₁ₐ and R₆ are as described above, and in some cases, R₃ is halo, O(C₁-C₆alkyl) or O(C₁-C₆)haloalkyl; or R₃ is F, Cl, methoxy, ethoxy, propoxy, butoxy, isopropoxy, isobutyloxy or OCF₃. For any compound of formula (I-Z-3-a), R₁ₐ, R₆ and R₃ are as described above, and in some cases, R₅ is hydrogen or F or Cl.

For any compound of formula (I-Z-3-d), R₁ₐ and R₆ are as described above, and in some cases, R₄ is halo, O(C₁-C₆alkyl) or O(C₁-C₆)haloalkyl; or R₄ is F, Cl, methoxy, ethoxy, propoxy, butoxy, isopropoxy, isobutyloxy or OCF₃.

In one aspect, provided herein is a compound selected from
(3Z)-3-[(3-methyl-1H-pyrazol-5-yl)methylene]indolin-2-one
(3Z)-5-methoxy-3-[(2-methyl-1H-imidazol-5-yl)methylene]indolin-2-one
(3Z)-3-(1H-pyrrolo[2,3-b]pyridin-2-ylmethylene)indolin-2-one
(3E)-3-(1,3-benzoxazol-2-ylmethylene)indolin-2-one
(3Z)-6-methoxy-3-[(2-methyl-1H-imidazol-5-yl)methylene]indolin-2-one
(3E)-3-(1,3-benzothiazol-2-ylmethylene)indolin-2-one
(3E)-3-(3-quinolylmethylene)indolin-2-one
(3Z)-5-chloro-3-[(2-methyl-1H-imidazol-5-yl)methylene]indolin-2-one
(3Z)-3-[(2-methyl-1H-imidazol-5-yl)methylene]-5-(trifluoromethoxy)indolin-2-one
(3Z)-3-[(2-isopropyl-1H-imidazol-5-yl)methylene]-5-methoxy-indolin-2-one
(3Z)-5-ethoxy-3-[(2-methyl-1H-imidazol-5-yl)methylene]indolin-2-one
(3Z)-3-[(2-methyl-1H-imidazol-5-yl)methylene]-5-methylsulfonyl-indolin-2-one
(3Z)-3-[(2-ethyl-1H-imidazol-5-yl)methylene]-5-methoxy-indolin-2-one
(3Z)-5-methoxy-3-[[2-(methoxymethyl)-1H-imidazol-5-yl]methylene]indolin-2-one
(3Z)-3-[(2-tert-butyl-1H-imidazol-5-yl)methylene]-5-methoxy-indolin-2-one
(3Z)-3-[[2-(2,6-difluorophenyl)-1H-imidazol-5-yl]methylene]-5-methoxy-indolin-2-one
(3Z)-5-isopropoxy-3-[(2-isopropyl-1H-imidazol-5-yl)methylene]indolin-2-one
(3Z)-3-[(2-isobutyl-1H-imidazol-5-yl)methylene]-5-isopropoxy-indolin-2-one
(3Z)-5-isopropoxy-3-[[2-(morpholinomethyl)-1H-imidazol-5-yl]methylene]indolin-2-one
(3Z)-5-methoxy-3-[[2-(2-methoxyethyl)-1H-imidazol-5-yl]methylene]indolin-2-one
(3Z)-5-isopropoxy-3-[(5-isopropyl-4H-1,2,4-triazol-3-yl)methylene]indolin-2-one
(3Z)-5-isopropoxy-3-[(2-morpholino-1H-imidazol-5-yl)methylene]indolin-2-one
(3Z)-5-hydroxy-3-[(2-tetrahydropyran-4-yl-1H-imidazol-5-yl)methylene]indolin-2-one
(3Z)-5-ethyl-3-[(2-tetrahydropyran-4-yl-1H-imidazol-5-yl)methylene]indolin-2-one
(3Z)-5-isopropoxy-3-[(2-tetrahydropyran-4-yl-1H-imidazol-5-yl)methylene]indolin-2-one
(3Z)-3-[[2-[1-(2-fluoroethyl)-4-piperidyl]-1H-imidazol-5-yl]methylene]-5-isopropoxy-indolin-2-one
(3Z)-5-chloro-3-[(2-tetrahydropyran-4-yl-1H-imidazol-5-yl)methylene]indolin-2-one
(3Z)-5-chloro-3-[[2-[1-(2-fluoroethyl)-4-piperidyl]-1H-imidazol-5-yl]methylene]indolin-2-one
(3Z)-5-(2-fluoroethoxy)-3-[(2-tetrahydropyran-4-yl-1H-imidazol-5-yl)methylene]indolin-2-one
(3Z)-3-[[2-(dimethylamino)-1H-imidazol-5-yl]methylene]-5-isopropoxy-indolin-2-one
(3Z)-7-fluoro-5-isopropoxy-3-[(2-morpholino-1H-imidazol-5-yl)methylene]indolin-2-one
(3Z)-6-fluoro-5-isopropoxy-3-[(2-morpholino-1H-imidazol-5-yl)methylene]indolin-2-one
(3Z)-4-fluoro-5-isopropoxy-3-[(2-morpholino-1H-imidazol-5-yl)methylene]indolin-2-one
(3Z)-5-isopropoxy-3-[1-(2-morpholino-1H-imidazol-5-yl)ethylidene]indolin-2-one
(3Z)-6-fluoro-5-isopropoxy-3-[(2-tetrahydropyran-4-yl-1H-imidazol-5-yl)methylene]indolin-2-one
(3Z)-6-fluoro-5-isopropoxy-3-[1-(2-morpholino-1H-imidazol-5-yl)ethylidene]indolin-2-one
(3Z)-6-fluoro-3-[[2-[1-(2-fluoroethyl)-4-piperidyl]-1H-imidazol-5-yl]methylene]-5-isopropoxy-indolin-2-one and
(3Z)-3-[[2-(4-ethylpiperazin-1-yl)-1H-imidazol-5-yl]methylene]-6-fluoro-5-isopropoxy-indolin-2-one.

In one aspect, provided herein is a compound of structure
(3Z)-5-isopropoxy-3-[(5-tetrahydropyran-4-yl-1H-imidazol-2-yl)methylene]indolin-2-one.

In one aspect, provided herein is a compound selected from
(3Z)-5-methoxy-3-[(2-methyl-1H-imidazol-5-yl)methylene]indolin-2-one
(3Z)-6-methoxy-3-[(2-methyl-1H-imidazol-5-yl)methylene]indolin-2-one
(3Z)-5-chloro-3-[(2-methyl-1H-imidazol-5-yl)methylene]indolin-2-one
(3Z)-3-[(2-methyl-1H-imidazol-5-yl)methylene]-5-(trifluoromethoxy)indolin-2-one
(3Z)-3-[(2-isopropyl-1H-imidazol-5-yl)methylene]-5-methoxy-indolin-2-one
(3Z)-5-ethoxy-3-[(2-methyl-1H-imidazol-5-yl)methylene]indolin-2-one
(3Z)-3-[(2-methyl-1H-imidazol-5-yl)methylene]-5-methylsulfonyl-indolin-2-one
(3Z)-3-[(2-ethyl-1H-imidazol-5-yl)methylene]-5-methoxy-indolin-2-one
(3Z)-5-methoxy-3-[[2-(methoxymethyl)-1H-imidazol-5-yl]methylene]indolin-2-one
(3Z)-3-[(2-tert-butyl-1H-imidazol-5-yl)methylene]-5-methoxy-indolin-2-one
(3Z)-3-[[2-(2,6-difluorophenyl)-1H-imidazol-5-yl]methylene]-5-methoxy-indolin-2-one
(3Z)-5-isopropoxy-3-[(2-isopropyl-1H-imidazol-5-yl)methylene]indolin-2-one
(3Z)-3-[(2-isobutyl-1H-imidazol-5-yl)methylene]-5-isopropoxy-indolin-2-one
(3Z)-5-isopropoxy-3-[[2-(morpholinomethyl)-1H-imidazol-5-yl]methylene]indolin-2-one
(3Z)-5-methoxy-3-[[2-(2-methoxyethyl)-1H-imidazol-5-yl]methylene]indolin-2-one
(3Z)-5-isopropoxy-3-[(2-morpholino-1H-imidazol-5-yl)methylene]indolin-2-one
(3Z)-5-hydroxy-3-[(2-tetrahydropyran-4-yl-1H-imidazol-5-yl)methylene]indolin-2-one
(3Z)-5-ethyl-3-[(2-tetrahydropyran-4-yl-1H-imidazol-5-yl)methylene]indolin-2-one
(3Z)-5-isopropoxy-3-[(2-tetrahydropyran-4-yl-1H-imidazol-5-yl)methylene]indolin-2-one
(3Z)-3-[[2-[1-(2-fluoroethyl)-4-piperidyl]-1H-imidazol-5-yl]methylene]-5-isopropoxy-indolin-2-one
(3Z)-5-chloro-3-[(2-tetrahydropyran-4-yl-1H-imidazol-5-yl)methylene]indolin-2-one
(3Z)-5-chloro-3-[[2-[1-(2-fluoroethyl)-4-piperidyl]-1H-imidazol-5-yl]methylene]indolin-2-one
(3Z)-5-(2-fluoroethoxy)-3-[(2-tetrahydropyran-4-yl-1H-imidazol-5-yl)methylene]indolin-2-one
(3Z)-3-[[2-(dimethylamino)-1H-imidazol-5-yl]methylene]-5-isopropoxy-indolin-2-one
(3Z)-7-fluoro-5-isopropoxy-3-[(2-morpholino-1H-imidazol-5-yl)methylene]indolin-2-one
(3Z)-6-fluoro-5-isopropoxy-3-[(2-morpholino-1H-imidazol-5-yl)methylene]indolin-2-one
(3Z)-4-fluoro-5-isopropoxy-3-[(2-morpholino-1H-imidazol-5-yl)methylene]indolin-2-one
(3Z)-5-isopropoxy-3-[1-(2-morpholino-1H-imidazol-5-yl)ethylidene]indolin-2-one
(3Z)-6-fluoro-5-isopropoxy-3-[(2-tetrahydropyran-4-yl-1H-imidazol-5-yl)methylene]indolin-2-one
(3Z)-6-fluoro-5-isopropoxy-3-[1-(2-morpholino-1H-imidazol-5-yl)ethylidene]indolin-2-one
(3Z)-6-fluoro-3-[[2-[1-(2-fluoroethyl)-4-piperidyl]-1H-imidazol-5-yl]methylene]-5-isopropoxy-indolin-2-one and
(3Z)-3-[[2-(4-ethylpiperazin-1-yl)-1H-imidazol-5-yl]methylene]-6-fluoro-5-isopropoxy-indolin-2-one.

In another aspect, provided herein is a pharmaceutical composition comprising any compound of any formula described above, and herein (e.g., compounds of formula (I), formula (II), formula (I-D-1), formula (I-D-2), formula (I-I-1), formula (I-I-2), formula (I-Z-1), formula (I-Z-2), formula (I-Z-3-a), formula (I-Z-3-b), formula (I-Z-3-c), or formula (I-Z-3-d)), and a pharmaceutically acceptable carrier, excipient, or binder.

Also provided herein is a method of treating an individual suffering from or susceptible to a neurodegenerative disease comprising administration of any compound of any formula described above, and herein, (e.g., compounds of formula (I), formula (II), formula (I-D-1), formula (I-D-2), formula (I-I-1), formula (I-I-2), formula (I-Z-1), formula (I-Z-2), formula (I-Z-3-a), formula (I-Z-3-b), formula (I-Z-3-c), or formula (I-Z-3-d)), to the individual in need thereof. In some of such embodiments, the neurodegenerative disease is selected from Parkinson's disease, Huntington's disease, Alzheimer's disease, multiple sclerosis or amyotrophic lateral sclerosis. In some embodiments, the neurodegenerative disease is Parkinson's disease.

Provided herein is a method for inhibiting a leucine-rich repeat kinase-2 (LRRK2) kinase, the method comprising contacting an LRRK2 kinase with a compound of any formula described above, and herein (e.g., compounds of formula (I), formula (II), formula (I-D-1), formula (I-D-2), formula (I-I-1), formula (I-I-2), formula (I-Z-1), formula (I-Z-2), formula (I-Z-3-a), formula (I-Z-3-b), formula (I-Z-3-c), or formula (I-Z-3-d)).

Provided herein is a method for treating a disorder or condition that is treated by inhibiting LRRK2 activity in a subject in need of treatment thereof, the method comprising administering to the subject a therapeutically effective amount of any compound of any formula described above, and herein (e.g., compounds of formula (I), formula (II), formula (I-D-1), formula (I-D-2), formula (I-I-1), formula (I-I-2), formula (I-Z-1), formula (I-Z-2), formula (I-Z-3-a), formula (I-Z-3-b), formula (I-Z-3-c), or formula (I-Z-3-d)).

In some embodiments of the methods described above, the compound is radiolabeled.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows percent cell death upon treatment with a compound of Formula I in a neuroprotection assay which is described in Example 4. Primary neurons were transfected with G2019S mutant (or wild-type) LRRK2 and treated with a compound of Formula I at the indicated concentrations. GFP is a control. The compound shows a neuroprotective effect in this assay.

### DETAILED DESCRIPTION OF THE INVENTION

Neurodegenerative disorders commonly afflict individuals over the age of 65. While current therapeutic approaches allow for management of symptoms early in the disease course, there is an inevitable, and fatal, progression of disease.

For example, Parkinson's disease is a common late-onset neurodegenerative disease affecting nearly 2% of individuals over the age of 65 years old. There is no cure for Parkinson's disease (PD) although symptoms can be managed early in the disease course through dopamine replacement. The oral drug, L-DOPA, a dopamine precursor, identified 50 years ago, remains the front-line treatment for PD (˜80% market share). This medication can be an effective symptomatic treatment in the early stages of the disease. However, L-DOPA is not a cure, and does not alter the underlying neurobiology of the disease. It only decreases the severity of some of the motor signs, and becomes less effective over time. For advanced disease, deep brain stimulation, in which a battery operated device is implanted to stimulate the affected areas of the brain can improve some of the motor symptoms. However, such treatment while effective, is highly invasive and has been shown to cause more adverse events than drug therapy.

Accordingly, there is an unmet need for neuroprotective therapy for patients suffering from debilitating neurodegenerative diseases. Described herein are novel methods for treatment of CNS disorders including neurodegenerative disorders. Also provided herein is a small molecule therapeutic approach for treatment of neurodegenerative disorders comprising targeting an enzyme that has been shown to be mutated in individuals suffering from neurodegenerative disorders. Mutations in the LRRK enzymes cause increased kinase activity; such increased kinase activity is associated with neurodegenerative disorders.

### Leucine Rich Repeat Kinases (LRRKs)

LRRK2 is a part of the leucine rich repeat kinase family and is a very large multidomain protein of 2527 amino acids, with two enzymatic domains: a protein kinase domain and a catalytic domain termed Roc (Ras in complex proteins), belonging to the Ras/GTPase family. There is also a WD40 domain and a COR domain (C terminal of Roc), an ankyrin repeat region, and a leucine-rich repeat (LRR), consisting of twelve repetitions of a 22-28 amino acid motif.

Point mutations are present in almost all of the identified domains. For instance the I1122V mutation is in the LRR domain; the R1441C mutation is in the Roc domain; the Y1699C mutation is in the COR domain, and the 12020T and G2019S mutations are in the kinase domain. The features of distribution of mutations in several different domains, and the lack of deletions or truncations, along with dominant inheritance, are consistent with a gain of function mechanism.

Different autosomal dominant point mutations within the gene encoding for the Leucine Rich Repeat protein Kinase-2 (LRRK2) can predispose humans to develop late-onset Parkinson's disease (PD, OMIM accession number 609007), with a clinical appearance indistinguishable from idiopathic PD. Data indicates that mutations in LRRK2 can be relatively frequent, not only accounting for 5-10% of familial PD, but are also found in a significant proportion of sporadic PD cases. Little is known about how LRRK2 is regulated in cells, what are its physiological substrates and how mutations in LRRK2 cause or increase risk of PD. In mammals there are two isoforms of the LRRK protein kinase, LRRK1 (2038 residues) and LRRK2 (2527 residues). They belong to a protein family that has also been termed Roco. Thus far mutations in LRRK2, but not LRRK1 have been linked to PD.

The most prevalent mutant form of LRRK2 (G2019S) accounting for approximately 6% of familial PD and 3% of sporadic PD cases in Europe, comprises an amino acid substitution of Gly2019 located within the conserved DYG-Mg2+-binding motif, in subdomain-VII of the kinase domain, to a Ser residue. There are suggestions that this mutation moderately enhances, approximately 2-3-fold, the autophosphorylation of LRRK2, as well as its ability to phosphorylate myelin basic protein. These findings suggest that over-activation of LRRK2 predisposes humans to develop PD. The study of LRRK2 has been hampered by the difficulty in expressing active recombinant enzyme and by the lack of a robust quantitative assay.

Accordingly, provided herein are modulators of LRRK kinases. In some embodiments, a modulator of LRRK is LRRK agonist. In some other embodiments, a modulator of LRRK is an inhibitor or partial inhibitor of a LRRK. In some embodiments, the compounds described herein are selective inhibitors of LRRK2. In some embodiments, the compounds described herein are pan inhibitors of the LRRKs. In some embodiments, high potency LRRK2 kinase inhibitors that cross the blood brain barrier provide a therapeutic advance in treatment of CNS disorders including neurodegenerative disorders. In some embodiments, the small molecule inhibitors of LRRK2 provided herein and the methods of treatment described herein slow down, stop or reverse the progression of a neurodegenerative disease. In some embodiments, compounds of formula (I), formula (II), formula (I-D-1), formula (I-D-2), formula (I-I-1), formula (I-I-2), formula (I-Z-1), formula (I-Z-2), formula (I-Z-3-a), formula (I-Z-3-b), formula (I-Z-3-c), or formula (I-Z-3-d), inhibit or partially inhibit the activity of mutated LRRK-2 (e.g., G2019S mutated form of LRRK-2). As shown in Example 3, Table 3, the compounds described herein are selective for LRKKs over other kinases.

The section headings used herein are for organizational purposes only and are not to be construed as limiting the subject matter described.

### CERTAIN DEFINITIONS

Unless defined otherwise, all technical and scientific terms used herein have the same meaning as is commonly understood by one of skill in the art to which the claimed subject matter belongs. In the event that there is a plurality of definitions for terms herein, those in this section prevail.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/or” unless stated otherwise. Furthermore, use of the term “including” as well as other forms, such as “include”, “includes”, and “included” is not limiting.

Definition of standard chemistry terms may be found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology, within the skill of the art are employed. Unless specific definitions are provided, the nomenclature employed herein are the standard definitions. Standard techniques can be used for chemical syntheses, chemical analyses, pharmaceutical preparation, formulation, and delivery, and treatment of individuals. Reactions and purification techniques can be performed e.g., using kits of manufacturer's specifications or as commonly accomplished in the art or as described herein. The foregoing techniques and procedures can be generally performed of conventional methods well known in the art and as described in various general and more specific references that are cited and discussed throughout the present specification. Throughout the specification, groups and substituents thereof can be chosen by one skilled in the field to provide stable moieties and compounds.

Where substituent groups are specified by their conventional chemical formulas, written from left to right, they equally encompass the chemically identical substituents that would result from writing the structure from right to left. As a non-limiting example, —CH₂O— is equivalent to —OCH₂—.

Unless otherwise noted, the use of general chemical terms, such as, though not limited to, “alkyl,” “amine,” “aryl,” can include their optionally substituted forms. For example, “alkyl,” as used herein, includes optionally substituted alkyl.

The terms “moiety”, “substituent”, “group” and “chemical group”, as used herein refer to a specific segment or functional group of a molecule. Chemical substituents are often recognized chemical entities embedded in or appended to a molecule.

The term “reactant,” as used herein, refers to a nucleophile or electrophile used to create covalent linkages.

The term “bond” or “single bond” refers to a chemical bond between two atoms, or two moieties when the atoms joined by the bond are considered to be part of larger substructure.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined below. For example, “optionally substituted aryl” means either unsubstituted aryl (e.g., a phenyl) or a substituted aryl (e.g., phenyl with 1-6 substitutents as described below). Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

In certain non-limiting examples, “optionally substituted” indicates that the group is optionally substituted with alkyl, alkenyl, alkynyl, heteroalkyl, heteroalkenyl, heteroalkynyl, haloalkyl, haloalkenyl, haloalkynyl, perhaloalkyl, halo, cycloalkyl, cycloalkenyl, heteroalicycl, aryl, heteroaryl, carbocycl, heterocycl, hydroxy, alkoxy, cyano, cyanoalkyl, carboxyl, sulfhydryl, amino, an amino acid, fused cycloalkyl, spiro cycloalkyl, fused heteroaryl, fused aryl, sulfonyl, sulfinyl, sulfonamidyl, sulfamidyl, phoshonate ester, amido, ether, alkylester, oxo, or combinations thereof. In specific instances, a group designated as “optionally substituted” indicates that the group is optionally substituted with hydrogen, hydroxy, nitro, cyano, methylthiol, thiol, azido, methyl, ethyl, propyl, iso-propyl, n-butyl, iso-butyl, sec-butyl, tert-butyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-pentyl, iso-pentyl, neo-pentyl, tert-amyl, hexyl, heptyl, octyl. ethenyl (—CH═CH₂), 1-propenyl (—CH₂CH═CH₂), isopropenyl [—C(CH₃)═CH₂], butenyl, 1,3-butadienyl, ethynyl, 2-propynyl, 2-butyryl, 1,3-butadiynyl, fluoro, chloro, bromo, iodo, fluoromethyl, difluoromethyl, trifluoromethyl, chloromethyl, dichloromethyl, trichloromethyl, bromomethyl, dibromomethyl, tribromomethyl, 1-chloro-1-fluoro-1-iodoethyl, fluroethyl, bromo ethyl, chloroethyl, iodoethyl, fluropropyl, bromopropyl, chloropropyl, iodopropyl, fluoroethenyl, chloroethenyl, bromo ethenyl, iodoethenyl, fluoro ethynyl chloro ethynyl, bromo ethynyl, iodoethynyl, trrifluoroethenyl, trichloroethenyl, tribromoethenyl, trifluoropropynyl, trichloropropynyl, tribromopropynyl, cyclopropyl, cyclobutyl, cyclopentyl, cyclohexyl, cycloheoptyl, spirocyclopropyl, spirocyclobutyl, spirocyclopentyl, pyridinyl, pyranyl, tetrahydrofuranyl, thiofuranyl, aziridinyl, oxiranyl, oxaziridinyl, dioxiranyl, azetidinyl, oxazyl, oxetanyl, theitanyl, pyrrolidinyl, oxolanyl, thiolanyl, oxazolidinyl, thiazolidinyl, decalinyl, bicyclo[2.2.1]heptyl, adamantly, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, quinolizinyl, cyclohexenyl, cyclopentadienyl, bicyclo[2.2.1]hept-2-ene, methoxy, ethoxy, n-propoxy, isopropoxy, n-butoxy, iso-butoxy, sec-butoxy, tert-butoxy, furanyl, thienyl, acridinyl, phenyl, benzyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazinyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl, pyridyl-N-oxide, methyl sulfonyl, ethyl sulfonyl, aminosulfonyl, trifluoromethyl sulfonyl, phosphinic acid, carboxylic acid, amido, amino, methylamine, ethylamine, dimethylamine, diethylamine, aminoethyldimethylamine, aminoethyldiethylamine, methylester, ethylester, propylester, isopropylester, butylester, or combinations thereof.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms, as well as the ranges C₁-C₂ and C₁-C₃. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “lower” as used herein in combination with terms such as alkyl, alkenyl or alkynyl, (i.e. “lower alkyl”, “lower alkenyl” or “lower alkynyl”) refers to an optionally substituted straight-chain, or optionally substituted branched-chain saturated hydrocarbon monoradical having from one to about six carbon atoms, more preferably one to three carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl.

The terms “heteroatom” or “hetero” as used herein, alone or in combination, refer to an atom other than carbon or hydrogen. In some embodiments, heteroatoms are independently selected from among oxygen, nitrogen, sulfur, phosphorous, silicon, selenium and tin but are not limited to these atoms. In embodiments in which two or more heteroatoms are present, the two or more heteroatoms can be the same as each another, or some or all of the two or more heteroatoms can each be different from the others.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “aliphatic” as used herein, alone or in combination, refers to an optionally substituted, straight-chain or branched-chain, non-cyclic, saturated, partially unsaturated, or fully unsaturated nonaromatic hydrocarbon. Thus, the term collectively includes alkyl, alkenyl and alkynyl groups.

The term “alkoxy” refers to optionally substituted O-alkyl groups where alkyl is as defined above. Examples of alkoxy include methoxy, ethoxy, n-propoxy, isopropoxy, n-butoxy, iso-butoxy, sec-butoxy, tert-butoxy and the like. Examples of substituted alkoxy include haloalkoxy, OCH₂(cyclopropyl) and the like.

The term “cycloalkyloxy” refers to optionally substituted O-cycloalkyl groups where cycloalkyl is as defined herein. Examples include O-(cyclopropyl), O-(cyclobutyl) or the like.

The term “heteroalkyl”, as used herein, alone or in combination, refers to optionally substituted alkyl structures, as described above, in which one or more of the skeletal chain carbon atoms (and any associated hydrogen atoms, as appropriate) are each independently replaced with a heteroatom (i.e. an atom other than carbon, such as though not limited to oxygen, nitrogen, sulfur, silicon, phosphorous, tin or combinations thereof), or heteroatomic group such as though not limited to —O—O—, —S—S—, —O—S—, —S—O—, ═N—N═, —N═N—, —N═N—NH—, —P(O)₂—, —O—P(O)₂—, —P(O)₂—O—, —S(O)—, —S(O)₂—, —SnH₂— and the like. Certain non-limiting examples of heteroalkyl groups include —CH₂OCH₂CH₂NH₂, —CH₂CH₂NHCH₂CH₂OH, —CH₂CH₂N(Me)CH₂CH₂OH, —CH₂OCH₂CH₂N(CH₃)₂, —CH₂NHCH₂CH₂OCH₃, —CH₂NHCH₂CH(cyclopropyl)CH₂OCH₃ and the like.

The terms “haloalkyl”, as used herein, alone or in combination, refer to optionally substituted alkyl groups, as defined above, in which one or more hydrogen atoms is replaced by fluorine, chlorine, bromine or iodine atoms, or combinations thereof. In some embodiments two or more hydrogen atoms may be replaced with halogen atoms that are the same as each another (e.g. difluoromethyl); in other embodiments two or more hydrogen atoms may be replaced with halogen atoms that are not all the same as each other (e.g. 1-chloro-1-fluoro-1-iodoethyl). Non-limiting examples of haloalkyl groups are fluoromethyl and bromoethyl. A non-limiting example of a haloalkenyl group is bromoethenyl. A non-limiting example of a haloalkynyl group is chloroethynyl.

The term haloalkoxy refers to optionally substituted O-haloalkyl groups where haloalkyl is as defined above.

The term “perhalo” as used herein, alone or in combination, refers to groups in which all of the hydrogen atoms are replaced by fluorines, chlorines, bromines, iodines, or combinations thereof. Thus, as a non-limiting example, the term “perhaloalkyl” refers to an alkyl group, as defined herein, in which all of the H atoms have been replaced by fluorines, chlorines, bromines or iodines, or combinations thereof. A non-limiting example of a perhaloalkyl group is bromo, chloro, fluoromethyl.

The term “carbon chain” as used herein, alone or in combination, refers to any alkyl, alkenyl, alkynyl, heteroalkyl, heteroalkenyl or heteroalkynyl group, which is linear, cyclic, or any combination thereof. If the chain is part of a linker and that linker comprises one or more rings as part of the core backbone, for purposes of calculating chain length, the “chain” only includes those carbon atoms that compose the bottom or top of a given ring and not both, and where the top and bottom of the ring(s) are not equivalent in length, the shorter distance shall be used in determining the chain length. If the chain contains heteroatoms as part of the backbone, those atoms are not calculated as part of the carbon chain length.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. Rings can be optionally substituted. Rings can form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “fused” as used herein, alone or in combination, refers to cyclic structures in which two or more rings share one or more bonds.

The term “cycloalkyl” as used herein, alone or in combination, refers to an optionally substituted, saturated, hydrocarbon monoradical ring, containing from three to about fifteen ring carbon atoms or from three to about ten ring carbon atoms, though may include additional, non-ring carbon atoms as substituents (e.g. methylcyclopropyl). Whenever it appears herein, a numerical range such as “C₃-C₆ cycloalkyl” or “C₃₋₆ cycloalkyl”, means that the cycloalkyl group may consist of 3 carbon atoms, 4 carbon atoms, 5 carbon atoms or 6 carbon atoms, i.e., is cyclopropyl, cyclobutyl, cyclopentyl or cycloheptyl, although the present definition also covers the occurrence of the term “cycloalkyl” where no numerical range is designated. The term includes fused, non-fused, bridged and spiro radicals. A fused cycloalkyl may contain from two to four fused rings where the ring of attachment is a cycloalkyl ring, and the other individual rings may be alicyclic, heterocyclic, aromatic, heteroaromatic or any combination thereof. Examples include, but are not limited to cyclopropyl, cyclopentyl, cyclohexyl, decalinyl, and bicyclo[2.2.1]heptyl and adamantyl ring systems. Illustrative examples include, but are not limited to the following moieties:

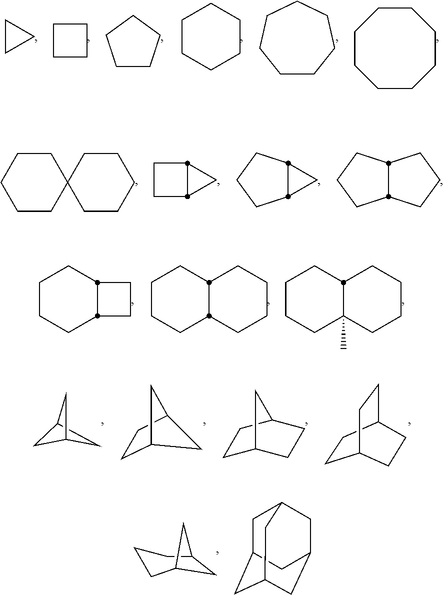

and the like.

The term “cycloalkenyl” as used herein, alone or in combination, refers to an optionally substituted hydrocarbon non-aromatic, monoradical ring, having one or more carbon-carbon double-bonds and from three to about twenty ring carbon atoms, three to about twelve ring carbon atoms, or from three to about ten ring carbon atoms. The term includes fused, non-fused, bridged and spiro radicals. A fused cycloalkenyl may contain from two to four fused rings where the ring of attachment is a cycloalkenyl ring, and the other individual rings may be alicyclic, heterocyclic, aromatic, heteroaromatic or any combination thereof. Fused ring systems may be fused across a bond that is a carbon-carbon single bond or a carbon-carbon double bond. Examples of cycloalkenyls include, but are not limited to cyclohexenyl, cyclopentadienyl and bicyclo[2.2.1]hept-2-ene ring systems. Illustrative examples include, but are not limited to the following moieties:

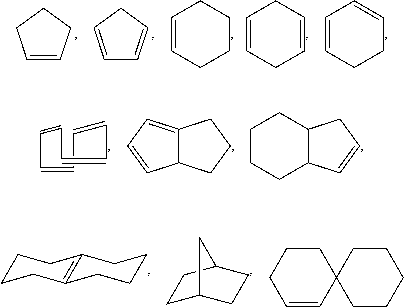

and the like.

The terms “alicyclyl” or “alicyclic” as used herein, alone or in combination, refer to an optionally substituted, saturated, partially unsaturated, or fully unsaturated nonaromatic hydrocarbon ring systems containing from three to about twenty ring carbon atoms, three to about twelve ring carbon atoms, or from three to about ten ring carbon atoms. Thus, the terms collectively include cycloalkyl and cycloalkenyl groups.

The terms “non-aromatic heterocyclyl” and “heteroalicyclyl” as used herein, alone or in combination, refer to optionally substituted, saturated, partially unsaturated, or fully unsaturated nonaromatic ring monoradicals containing from three to about twenty ring atoms, where one or more of the ring atoms are an atom other than carbon, independently selected from among oxygen, nitrogen, sulfur, phosphorous, silicon, selenium and tin but are not limited to these atoms. In embodiments in which two or more heteroatoms are present in the ring, the two or more heteroatoms can be the same as each another, or some or all of the two or more heteroatoms can each be different from the others. The terms include fused, non-fused, bridged and spiro radicals. A fused non-aromatic heterocyclic radical may contain from two to four fused rings where the attaching ring is a non-aromatic heterocycle, and the other individual rings may be alicyclic, heterocyclic, aromatic, heteroaromatic or any combination thereof. Fused ring systems may be fused across a single bond or a double bond, as well as across bonds that are carbon-carbon, carbon-hetero atom or hetero atom-hetero atom. The terms also include radicals having from three to about twelve skeletal ring atoms, as well as those having from three to about ten skeletal ring atoms. Attachment of a non-aromatic heterocyclic subunit to its parent molecule can be via a heteroatom or a carbon atom. Likewise, additional substitution can be via a heteroatom or a carbon atom. As a non-limiting example, an imidazolidine non-aromatic heterocycle may be attached to a parent molecule via either of its N atoms (imidazolidin-1-yl or imidazolidin-3-yl) or any of its carbon atoms (imidazolidin-2-yl, imidazolidin-4-yl or imidazolidin-5-yl). In certain embodiments, non-aromatic heterocycles contain one or more carbonyl or thiocarbonyl groups such as, for example, oxo- and thio-containing groups. Examples include, but are not limited to pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Illustrative examples of heterocycloalkyl groups, also referred to as non-aromatic heterocycles, include:

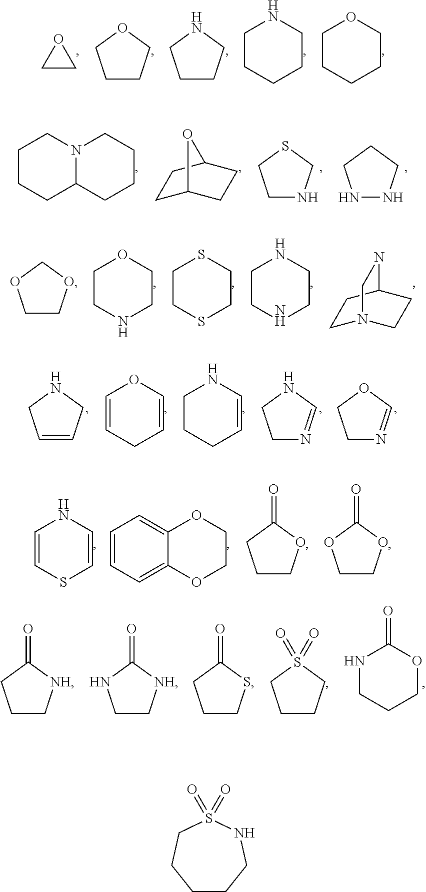

and the like.

The terms also include all ring forms of the carbohydrates, including but not limited to the monosaccharides, the disaccharides and the oligosaccharides.

The term “aromatic” as used herein, refers to a planar, cyclic or polycyclic, ring moiety having a delocalized π-electron system containing 4n+2π electrons, where n is an integer. Aromatic rings can be formed by five, six, seven, eight, nine, or more than nine atoms. Aromatics can be optionally substituted and can be monocyclic or fused-ring polycyclic. The term aromatic encompasses both all carbon containing rings (e.g., phenyl) and those rings containing one or more heteroatoms (e.g., pyridine).

The term “aryl” as used herein, alone or in combination, refers to an optionally substituted aromatic hydrocarbon radical of six to about twenty ring carbon atoms, and includes fused and non-fused aryl rings. A fused aryl ring radical contains from two to four fused rings where the ring of attachment is an aryl ring, and the other individual rings may be alicyclic, heterocyclic, aromatic, heteroaromatic or any combination thereof. Further, the term aryl includes fused and non-fused rings containing from six to about twelve ring carbon atoms, as well as those containing from six to about ten ring carbon atoms. A non-limiting example of a single ring aryl group includes phenyl; a fused ring aryl group includes naphthyl, phenanthrenyl, anthracenyl, azulenyl; and a non-fused bi-aryl group includes biphenyl.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidiazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazinyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl and the like, and their oxides, such as for example pyridyl-N-oxide. Illustrative examples of heteroaryl groups include the following moieties:

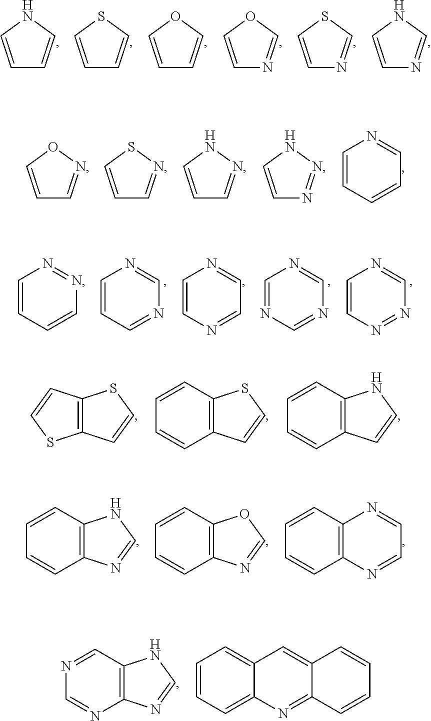

and the like.

The term “heterocyclyl” or “heterocycle” as used herein, alone or in combination, refers collectively to heteroalicyclyl and heteroaryl groups. Herein, whenever the number of carbon atoms in a heterocycle is indicated (e.g., C₁-C₆ heterocycle), at least one non-carbon atom (the heteroatom) must be present in the ring. Designations such as “C₁-C₆ heterocycle” refer only to the number of carbon atoms in the ring and do not refer to the total number of atoms in the ring. Designations such as “4-6 membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). For heterocycles having two or more heteroatoms, those two or more heteroatoms can be the same or different from one another. Heterocycles can be optionally substituted. Non-aromatic heterocyclic groups include groups having only three atoms in the ring, while aromatic heterocyclic groups must have at least five atoms in the ring. Bonding (i.e. attachment to a parent molecule or further substitution) to a heterocycle can be via a heteroatom or a carbon atom.

The term “carbocyclyl” as used herein, alone or in combination, refers collectively to alicyclyl and aryl groups; i.e. all carbon, covalently closed ring structures, which may be saturated, partially unsaturated, fully unsaturated or aromatic. Carbocyclic rings can be formed by three, four, five, six, seven, eight, nine, or more than nine carbon atoms. Carbocycles can be optionally substituted. The term distinguishes carbocyclic from heterocyclic rings in which the ring backbone contains at least one atom which is different from carbon. Carbocyclyl includes cycloalkyl and cycloalkenyl.

The terms “halogen”, “halo” or “halide” as used herein, alone or in combination refer to fluoro, chloro, bromo and iodo.

The term “hydroxy” as used herein, alone or in combination, refers to the monoradical —OH.

The term “carbonyl” as used herein, alone or in combination, refers to the diradical —C(═O)—, which may also be written as —C(O)—.

An “oxo” substituent refers to a (═O) substituent.

The terms “carboxy” or “carboxyl” as used herein, alone or in combination, refer to the moiety —C(O)OH, which may also be written as —COOH.

The term “sulfonyl” as used herein, alone or in combination, refers to the diradical —S(═O)₂—.

The terms “sulfonamide”, “sulfonamido” and “sulfonamidyl” as used herein, alone or in combination, refer to the diradical groups —S(═O)₂—NH— and —NH—S(═O)₂—.

The terms “sulfamide”, “sulfamido” and “sulfamidyl” as used herein, alone or in combination, refer to the diradical group —NH—S(═O)₂—NH—.

It is to be understood that in instances where two or more radicals are used in succession to define a substituent attached to a structure, the first named radical is considered to be terminal and the last named radical is considered to be attached to the structure in question. Thus, for example, the radical arylalkyl is attached to the structure in question by the alkyl group.

The term “patient”, “subject” or “individual” are used interchangeably. As used herein, they refer to individuals suffering from a disorder, and the like, encompasses mammals and non-mammals. None of the terms require that the individual be under the care and/or supervision of a medical professional. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the individual is a mammal. In preferred embodiments, the individual is a human.

The terms “treat, “treating” or “treatment,” and other grammatical equivalents as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease).

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In preferred embodiments, the compounds and compositions described herein are administered orally.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in a disease. An appropriate “effective” amount may differ from one individual to another. An appropriate “effective” amount in any individual case may be determined using techniques, such as a dose escalation study.

The term “acceptable” as used herein, with respect to a formulation, composition or ingredient, means having no persistent detrimental effect on the general health of the individual being treated.

The term “pharmaceutically acceptable” as used herein, refers to a material, such as a carrier or diluent, which does not abrogate the biological activity or properties of the compounds described herein, and is relatively nontoxic, i.e., the material may be administered to an individual without causing undesirable biological effects or interacting in a deleterious manner with any of the components of the composition in which it is contained.

The term “prodrug” as used herein, refers to a drug precursor that, following administration to an individual and subsequent absorption, is converted to an active, or a more active species via some process, such as conversion by a metabolic pathway. Thus, the term encompasses any derivative of a compound, which, upon administration to a recipient, is capable of providing, either directly or indirectly, a compound of this invention or a pharmaceutically active metabolite or residue thereof. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they may be easier to administer than the parent drug. They may, for instance, be bioavailable by oral administration whereas the parent is not. Particularly favored derivatives or prodrugs are those that increase the bioavailability of the compounds of this invention when such compounds are administered to an individual (e.g. by allowing an orally administered compound to be more readily absorbed into the blood) or which enhance delivery of the parent compound to a biological compartment (e.g. the brain or lymphatic system).

The term “pharmaceutically acceptable salt” as used herein, refers to salts that retain the biological effectiveness of the free acids and bases of the specified compound and that are not biologically or otherwise undesirable. Compounds described herein may possess acidic or basic groups and therefore may react with any of a number of inorganic or organic bases, and inorganic and organic acids, to form a pharmaceutically acceptable salt. These salts can be prepared in situ during the final isolation and purification of the compounds of the invention, or by separately reacting a purified compound in its free base form with a suitable organic or inorganic acid, and isolating the salt thus formed.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, excipients and the like.

The term “carrier” as used herein, refers to relatively nontoxic chemical compounds or agents that facilitate the incorporation of a compound into cells or tissues.

As used herein, the term “inhibitor” refers to a compound which inhibits or partially inhibits one or more kinases described herein. For example, the term “LRRK-2 inhibitor” refers to a compound which inhibits or partially inhibits the LRRK-2 receptor and/or reduces the signaling effect.

As used herein, the term “LRRK2-mediated disease” or a “disorder or disease or condition mediated by inappropriate LRRK2 activity” refers to any disease state mediated or modulated by LRRK2 kinase mechanisms. Such disease states include, but are not limited to, Parkinson's disease, Alzheimer's disease, Huntingtons diseases, and the like, or other neurodegenerative disease or a condition associated with aberrantly increased activity of LRRKs.

As used herein, the term “isotopic variant” refers to a compound that contains unnatural proportions of isotopes at one or more of the atoms that constitute such compound. In some embodiments, an “isotopic variant” of a compound contains one or more non-radioactive isotopes, such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art. In certain embodiments, the disclosed compounds include the preparation of isotopic variants with radioisotopes, in the instance for example, where the resulting compounds is used for drug and/or substrate tissue distribution studies. The radioactive isotopes tritium, i.e. ³H, and carbon-14, i.e. ¹⁴C, are particularly useful for this purpose in view of their ease of incorporation and ready means of detection. In other embodiments, compounds are prepared that are substituted with positron emitting isotopes, such as ¹¹C, ¹⁸F, ¹⁵O and ¹³N, and are useful in Positron Emission Topography (PET) studies for examining substrate receptor occupancy.

All isotopic variants of the compounds provided herein, radioactive or not, are intended to be encompassed within the scope of the contemplated compounds.

### Compounds

Provided herein in some embodiments are compounds, or salts, tautomers, or prodrugs thereof, of Formula (II):

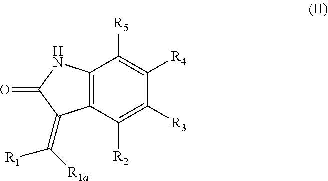

wherein
R₁ is a monocyclic or bicyclic heterocycle optionally substituted with one, two, three, four, or five R₆;
R₁ₐ is hydrogen, alkyl, heteroalkyl, cycloalkyl, or heteroalicyclyl;
each R₆ is independently hydroxy, halo, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₂, R₃, R₄, and R₅ are independently hydrogen, hydroxy, halo, alkyl, cycloalkyl, alkoxy, cycloalkyloxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted;
   where cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, cycloalkyl and heteroalicyclyl.

Provided herein in some embodiments are compounds, or salts, tautomers, or prodrugs thereof, of Formula (I):

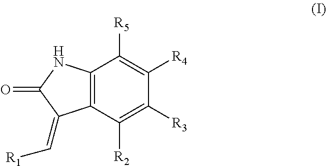

wherein
R₁ is a monocyclic or bicyclic heterocycle optionally substituted with one, two, three, four, or five R₆;
each R₆ is independently hydroxy, halo, haloalkyl, haloalkoxy, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
each R₂, R₃, R₄, and R₅ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted;
   where cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, cycloalkyl and heteroalicyclyl, and alkyl and alkoxy are independently, at each occurrence, optionally substituted with one or two groups selected from oxo, hydroxy, or amino.

Certain embodiments provided herein describe a compound of formula (I) having the structure of formula (I-A):

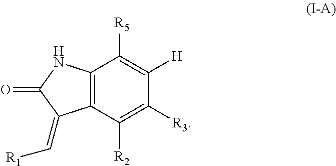

Provided herein in certain embodiments, is a compound represented by Formula (I-A), wherein R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₃ is C₁-C₆alkyl, C₁-C₆alkoxy, chloro or fluoro; and R₄ is chloro or fluoro.

Some embodiments provided herein describe a compound of formula (I) having the structure of formula (I-B):

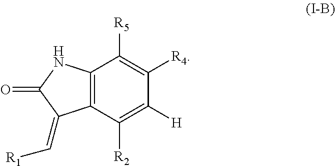

Some embodiments provided herein describe a compound of formula (I) having the structure of formula (I-C):

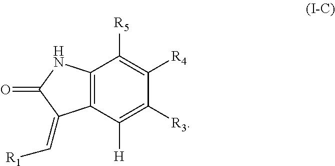

Some embodiments provided herein describe a compound of formula (I) having the structure of formula (I-D):

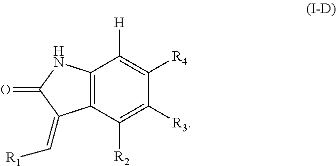

Provided herein in certain embodiments, is a compound represented by Formula (I-D), wherein R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₃ is hydrogen, C₁-C₆alkyl, C₁-C₆alkoxy, chloro or fluoro; and R₄ is hydrogen, chloro or fluoro. In certain specific embodiments, R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₃ is hydrogen; and R₄ is fluoro.

Other embodiments provided herein describe a compound of Formula (I-D), wherein R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₂ is hydrogen, C₁-C₆alkyl, C₁-C₆alkoxy, chloro or fluoro; and R₄ is hydrogen, chloro or fluoro. In certain specific embodiments, R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₂ is hydrogen; and R₄ is fluoro.

Some embodiments provided herein describe a compound of formula (I) having the structure of formula (I-E):

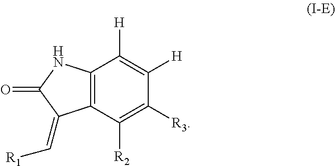

Provided herein in certain embodiments, is a compound represented by Formula (I-E), wherein R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₃ is C₁-C₆alkyl, C₁-C₆alkoxy, chloro or fluoro. In certain specific embodiments, R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₃ is fluoro.

In some embodiments of compounds described herein, the compound of formula (I) has the structure of formula (I-F):

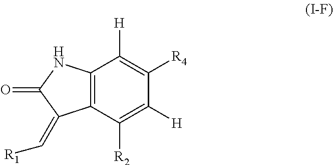

wherein
R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; and
R₄ is fluoro or chloro.

Some embodiments provided herein describe a compound of formula (I) having the structure of formula (I-F):

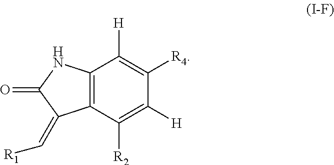

Provided herein in certain embodiments, is a compound represented by Formula (I-F), wherein R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₄ is C₁-C₆alkyl, C₁-C₆alkoxy, chloro or fluoro. In certain specific embodiments, R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₄ is fluoro.

Some embodiments provided herein describe a compound of formula (I) having the structure of formula (I-G):

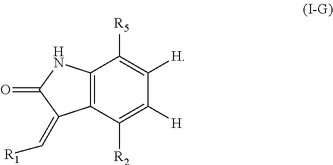

Provided herein in certain embodiments, is a compound represented by Formula (I-G), wherein R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₅ is C₁-C₆alkyl, C₁-C₆alkoxy, chloro or fluoro. In certain specific embodiments, R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₅ is fluoro.

In some embodiments of compounds described herein, the compound of formula (I) has the structure of formula (I-H):

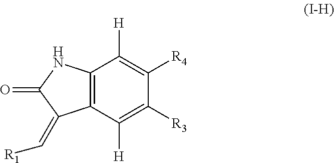

wherein
R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″, where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; and
R₄ is fluoro or chloro.

Some embodiments provided herein describe a compound of formula (I) having the structure of formula (I-H):

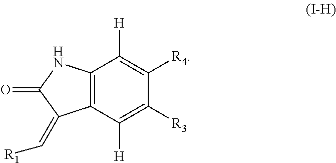

Certain embodiments provided herein describe a compound of Formula (I-H), wherein R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₄ is C₁-C₆alkyl, C₁-C₆alkoxy, chloro or fluoro. In certain specific embodiments, R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; and R₄ is fluoro.

Some embodiments provided herein describe a compound of formula (I) having the structure of formula (I-I):

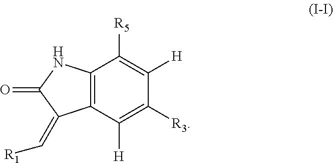

Certain embodiments provided herein describe a compound of Formula (I-I), wherein R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; R₅ is chloro or fluoro. In certain specific embodiments, R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; and R₅ is fluoro.

Some embodiments provided herein describe a compound of formula (I) having the structure of formula (I-J):

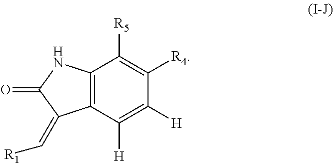

In some embodiments of compounds described herein, the compound of formula (I) has the structure formula (I-K):

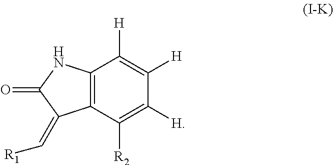

In some embodiments, R₂ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments, R₂ is hydroxy, F, Cl, CH₂F, CHF₂, CF₃, CH₂CH₂F, CH₂CHF₂, CH₂CF₃, methyl, ethyl, propyl, iso-propyl, cyclopropyl, methoxy, ethoxy, iso-propoxy, OCF₃, C(O)NMe₂, or SO₂Me. In some embodiments, R₂ is C(O)NMe₂ or SO₂Me.

In some embodiments, R₂ is S(O)₂NR′R″; and
R′ and R″ taken together with the nitrogen to which they are attached form a ring structure that optionally includes an additional heteroatom selected from N or O and is optionally substituted.

Some embodiments provided herein describe a compound of formula (I) having the structure of formula (I-K):

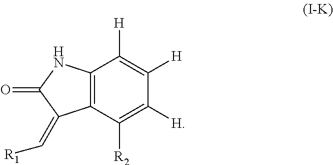

Provided herein in certain embodiments, is a compound represented by Formula (I-K), wherein R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted.

In some embodiments, the compound of formula (I) has the structure formula (I-L):

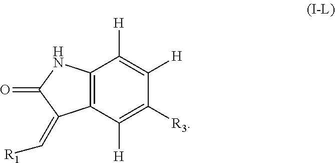

In some embodiments, R₃ is hydroxy, halo, haloC₁-C₆alkyl, haloC₁-C₆alkoxy, C₁-C₆alkyl, C₃-C₆cycloalkyl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments, R₃ is CF₃, CH₂CF₃, methyl, ethyl, iso-propyl, or cyclopropyl. In some embodiments, R₃ is methoxy, ethoxy, propoxy, iso-propoxy, or OCF₃, In some embodiments, R₃ is C(O)NMe₂ or SO₂Me.

In some embodiments, R₃ is C(O)NR′R″ or S(O)₂NR′R″; and
R′ and R″ taken together with the nitrogen to which they are attached form a ring structure that optionally includes an additional heteroatom selected from N or O and is optionally substituted.

Some embodiments provided herein describe a compound of formula (I) having the structure of formula (I-L):

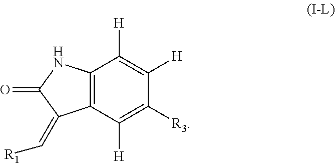

Certain embodiments provided herein describe a compound of Formula (I-L), wherein R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted.

Some embodiments provided herein describe a compound of formula (I) having the structure of formula (I-M):

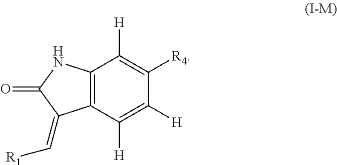

Certain embodiments provided herein describe a compound of Formula (I-M), wherein hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted.

In some embodiments, the compound of formula (I) has the structure formula (I-N):

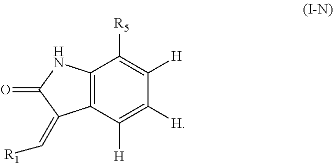

In some embodiments, R₅ is hydroxy, halo, haloC₁-C₃alkyl, or C₁-C₃alkyl. In some embodiments, R₅ is F or Cl. In some embodiments, R₁ is pyrazole, imidazole, triazole, triazolone, or benzimidazole, where R₁ is optionally substituted with one or two R₆. In some embodiments, R₁ is imidazole, triazole, triazolone, or benzimidazole, where R₁ is optionally substituted with one or two R₆.

Some embodiments provided herein describe a compound of formula (I) having the structure of formula (I-N):

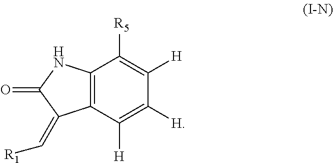

Provided herein in some embodiments is compound represented by any one of formulas (II), (I), (I-A), (I-B), (I-D), (LE), (I-F), (I-G), or (I-K), wherein R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes a heteroatom selected from N or O and is optionally substituted. In certain embodiments, R₂ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; where R′ and R″ taken together with the nitrogen to which they are attached form a ring structure that optionally includes an additional heteroatom selected from N or O and is optionally substituted. In certain specific embodiments, R₂ is hydroxy, F, Cl, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, C(O)NMe₂, or SO₂Me. In specific embodiments, R₂ is methoxy, ethoxy, iso-propoxy, or OCF₃, In further or additional embodiments, R₂ is C(O)NMe₂ or SO₂Me.

In some embodiments provided herein is a compound represented by any one of formulas (II), (I), (I-A), (I-B), (I-D), (I-E), (I-F), (I-G), or (I-K), wherein R₂ is S(O)₂NR′R″; where R′ and R″ taken together with the nitrogen to which they are attached form a ring structure that optionally includes an additional heteroatom selected from N or O and is optionally substituted. In some embodiments, the R′ and R″ taken together with the nitrogen to which they are attached form a four-membered, five-membered, six-membered, seven-membered, eight-membered, or nine-membered ring structure that is optionally substituted.

Provided herein in certain embodiments is a compound represented by any one of formulas (II), (I), (I-A), (I-C), (I-D), (I-E), (I-H), (I-I), or (I-L), wherein R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted.

Also provided herein in some embodiments is a compound represented by any one of formulas (II), (I), (I-A), (I-C), (I-D), (I-E), (I-H), (I-I), or (I-L), wherein R₃ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; where R′ and R″ taken together with the nitrogen to which they are attached form a ring structure that optionally includes an additional heteroatom selected from N or O and is optionally substituted. In certain embodiments, R₃ is hydroxy, CH₂F, CHF₂, CF₃, CH₂CH₂F, CH₂CHF₂, CH₂CF₃, methyl, ethyl, iso-propyl, cyclopropyl, cyclobutyl, cyclopentyl, methoxy, ethoxy, propoxy, iso-propoxy, OCF₃, C(O)NMe₂, or SO₂Me. In certain specific embodiments, R₃ is CF₃, CH₂CF₃, methyl, ethyl, iso-propyl, cyclopropyl, methoxy, ethoxy, propoxy, iso-propoxy, OCF₃, C(O)NMe₂, or SO₂Me. In further or additional embodiments, R₃ is C(O)NR′R″ or S(O)₂NR′R″; where R′ and R″ taken together with the nitrogen to which they are attached form a ring structure that optionally includes an additional heteroatom selected from N or O and is optionally substituted.

In some embodiments, provided herein is a compound represented by any one of formulas (I), (I-B), (I-C), (I-D), (I-F), (I-H), (I-J), or (I-M), wherein R₄ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted. In certain embodiments, R₄ is S(O)₂NR′R″; where R′ and R″ taken together with the nitrogen to which they are attached form a ring structure that optionally includes an additional heteroatom selected from N or O and is optionally substituted. In certain specific embodiments, R₄ is fluoro. In further or alternative embodiments, R₄ is chloro.

In some embodiments, provided herein is a compound represented by any one of formulas (II), (I), (I-A), (I-B), (I-C), (I-G), (I-I), (I-J), or (I-N), wherein R₅ is hydroxy, halo, haloC₁-C₃alkyl, or C₁-C₃alkyl. In certain embodiments, R₅ is hydroxy, F, Cl, Br, I, CH₂F, CHF₂, CF₃, CH₂CH₂F, CH₂CHF₂, CH₂CF₃, methyl, ethyl, propyl, or iso-propyl. In specific embodiments, R₅ is hydroxy, F, Cl, CF₃, or methyl. In further or additional embodiments, R₅ is hydroxy, F, or Cl. In further or alternative embodiments, R₅ is F. In further or alternative embodiments, R₅ is Cl.

In some embodiments, provided herein is a compound represented by any one of formulas (II), (I), (I-A), (I-B), (I-C), (I-D), (I-E), (I-F), (I-G), (I-H), (I-I), (I-J), (I-K), (I-L), (I-M), or (I-N), wherein R₁ is any heterocycle. In other embodiments, R₁ is a heterocycle containing one, two, three, or four nitrogen heteroatoms. In specific embodiments, R₁ is a heterocycle containing one nitrogen heteroatom. In specific embodiments, R₁ is a heterocycle containing two nitrogen heteroatoms. In specific embodiments, R₁ is a heterocycle containing three nitrogen heteroatoms. In certain embodiments, R₁ is a monocyclic heterocycle. In other embodiments, R₁ is a bicyclic heterocyle. In further or alternative embodiments, R₁ is a tricyclic heterocyle.

In further or additional embodiments, provided herein is a compound represented by any one of formulas (II), (I), (I-A), (I-B), (I-C), (I-D), (I-E), (I-F), (I-G), (I-H), (I-I), (I-J), (I-K), (I-L), (I-M), or (I-N), wherein R₁ is a heterocycle optionally substituted with R₆. In certain specific instances, R₁ is substituted with one R₆. In other specific embodiments, R₁ is substituted with two R₆. In certain embodiments, R₁ is substituted with three R₆. In particular embodiments, R₁ is substituted with four R₆. In some embodiments, R₁ is substituted with five R₆.

Certain embodiments, provided herein is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is any heteroaryl. In other embodiments, R₁ is a heteroaryl containing one, two, three, or four nitrogen heteroatoms. In specific embodiments, R₁ is a monocyclic, bicyclic, or tricyclic heteroaryl. In further or additional embodiments, R₁ is a heterocycle optionally substituted with one, two, three, four or five R₆.

In certain embodiments, provided herein is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is a heterocycle, wherein the heterocycle is pyrazole, pyrrole, imidazole, triazole, triazolone, indole, benzimidazole, azabenzimidazole, or azaindole. In further or alternative embodiments, R₁ is pyrazole, pyrrole, imidazole, triazole, triazolone, indole, benzimidazole, azabenzimidazole, or azaindole, where R₁ is optionally substituted. In other embodiments, R₁ is pyrazole, imidazole, triazole, triazolone, or benzimidazole, where R₁ is optionally substituted with one or two R₆. In some embodiments, R₁ is imidazole, triazole, triazolone, or benzimidazole, where R₁ is optionally substituted with one or two R₆.

Provided herein in some embodiments is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is imidazole optionally substituted with one, two, or three R₆. In certain specific embodiments, R₁ is imidazole substituted with one R₆. In other specific embodiments, R₁ is imidazole substituted with two R₆. In some embodiments, the imidazole is C-bound. In other embodiments, the imidazole is N-bound. In certain specific embodiments, the R₁ is an imidazole, wherein the imidazole has a structure of:

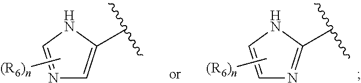

and

n is 0, 1, 2, or 3.

Provided herein in some embodiments is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is pyrazole optionally substituted with one, two, or three R₆. In certain specific embodiments, R₁ is pyrazole substituted with one R₆. In other specific embodiments, R₁ is pyrazole substituted with two R₆. In some embodiments, the pyrazole is C-bound. In other embodiments, the pyrazole is N-bound. In certain specific embodiments, the R₁ is a pyrazole, wherein the pyrazole has a structure of:

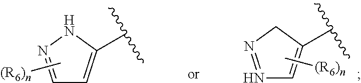

and

n is 0, 1, 2, or 3.

Provided herein in some embodiments is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is pyrrole optionally substituted with one, two, three, or four R₆. In certain specific embodiments, R₁ is pyrrole substituted with one R₆. In other specific embodiments, R₁ is pyrrole substituted with two R₆. In certain embodiments, R₁ is pyrrole substituted with three R₆. In some embodiments, the pyrrole is C-bound. In other embodiments, the pyrrole is N-bound. In certain specific embodiments, the R₁ is a pyrrole, wherein the pyrrole has a structure of:

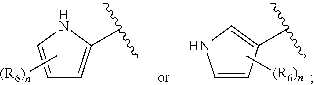

and

n is 0, 1, 2, 3, or 4.

Provided herein in some embodiments is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is triazole optionally substituted with one, two, or three R₆. In certain specific embodiments, R₁ is triazole substituted with one R₆. In other specific embodiments, R₁ is triazole substituted with two R₆. In some embodiments, the triazole is C-bound. In other embodiments, the triazole is N-bound. In certain specific embodiments, the R₁ is a triazole, wherein the triazole has a structure of:

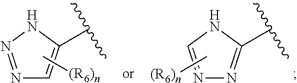

and

n is 0, 1, or 2.

Provided herein in some embodiments is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is triazolone optionally substituted with one, two, or three R₆. In certain specific embodiments, R₁ is triazolone substituted with one R₆. In other specific embodiments, R₁ is triazolone substituted with two R₆. In some embodiments, the triazolone is C-bound. In other embodiments, the triazolone is N-bound. In certain specific embodiments, the R₁ is a triazolone, wherein the triazolone has a structure of:

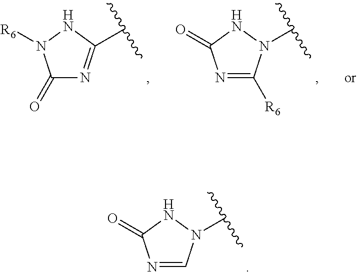

Provided herein in some embodiments is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is benzimidazole optionally substituted with one, two, three, four or five R₆. In certain specific embodiments, R₁ is benzimidazole substituted with one R₆. In other specific embodiments, R₁ is benzimidazole substituted with two R₆. In certain specific embodiments, R₁ is benzimidazole substituted with three R₆. In some embodiments, the benzimidazole is C-bound. In other embodiments, the benzimidazole is N-bound. In certain specific embodiments, the R₁ is a benzimidazole, wherein the benzimidazole has a structure of:

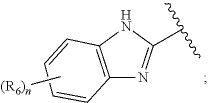

and

n is 0, 1, 2, 3, 4, or 5.

Provided herein in some embodiments is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is indole optionally substituted with one, two, three, four or five R₆. In certain specific embodiments, R₁ is indole substituted with one R₆. In other specific embodiments, R₁ is indole substituted with two R₆. In certain embodiments, R₁ is indole substituted with three R₆. In some embodiments, the indole is C-bound. In other embodiments, the indole is N-bound. In certain specific embodiments, the R₁ is an indole, wherein the indole has a structure of:

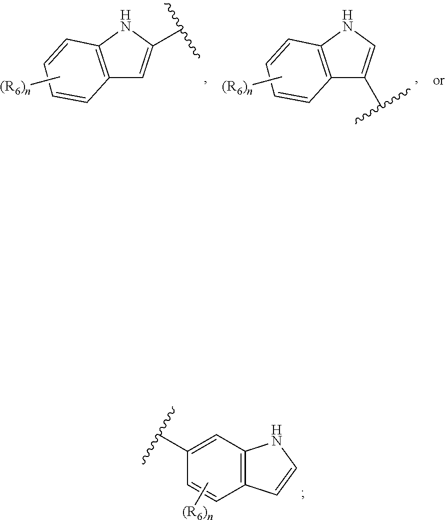

and

n is 0, 1, 2, 3, 4, or 5.

Provided herein in some embodiments is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₁ is azaindole optionally substituted with one, two, three, four or five R₆. In certain specific embodiments, R₁ is azaindole substituted with one R₆. In other specific embodiments, R₁ is azaindole substituted with two R₆. In certain embodiments, R₁ is azaindole substituted with three R₆. In some embodiments, the azaindole is C-bound. In other embodiments, the azaindole is N-bound. In certain specific embodiments, the R₁ is an azaindole, wherein the azaindole has a structure of:

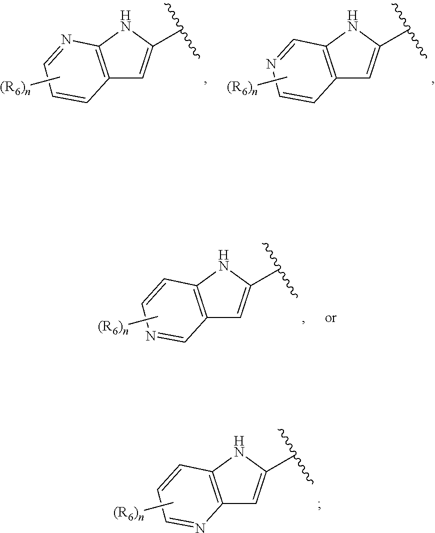

and

n is 0, 1, 2, 3, 4, or 5.

Provided herein in some embodiments is a compound represented by any one of formulas (II), (I), (I-A), (I-B), (I-C), (I-D), (I-E), (I-F), (I-G), (I-H), (I-I), (I-J), (I-K), (I-L), (I-M), or (I-N), wherein R₁ is azabenzimidazole optionally substituted with one, two, three, or four R₆. In certain specific embodiments, R₁ is azabenzimidazole substituted with one R₆. In other specific embodiments, R₁ is azabenzimidazole substituted with two R₆. In some embodiments, the azabenzimidazole is C-bound. In other embodiments, the azabenzimidazole is N-bound. In certain specific embodiments, the R₁ is an azabenzimidazole, wherein the azabenzimidazole has a structure of:

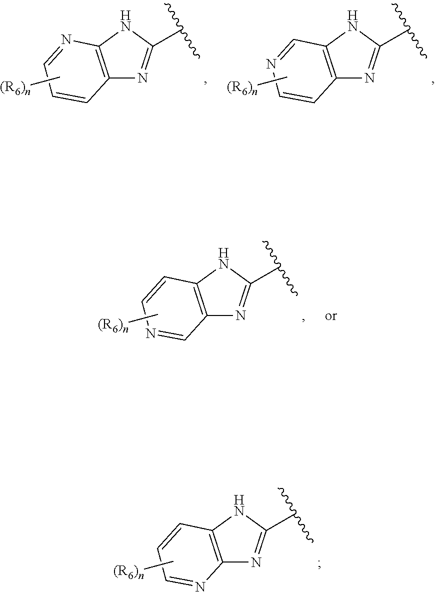

and

n is 0, 1, 2, 3, or 4.

In some embodiments, provided herein is a compound represented by any one of formulas (II), (I), (I-A), (I-B), (I-C), (I-D), (I-E), (I-F), (I-G), (I-H), (I-I), (I-J), (I-K), (I-L), (I-M), or (I-N), wherein R₆ is independently hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted. In certain embodiments, each R₆ is independently hydroxy, F, Cl, Br, I, CH₂F, CHF₂, CF₃, CH₂CH₂F, CH₂CHF₂, CH₂CF₃, methyl, ethyl, propyl, iso-propyl, butyl, sec-butyl, iso-butyl, tert-butyl, pentyl, hexyl, cyclopropyl, cyclobutyl, cyclopentyl, cyclohexyl, methoxy, ethoxy, propoxy, iso-propoxy, butoxy, phenyl, pyridyl, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, or SO₂NMe₂. In specific embodiments, R₆ is independently methyl, ethyl, propyl, iso-propyl, butyl, phenyl, SO₂Me, or C(O)NMe₂.

In further or alternative embodiments, provided herein is a compound represented by any one of formulas (II), (I), (I-A), (I-B), (I-C), (I-D), (LE), (I-F), (I-G), (I-H), (I-I), (I-J), (I-K), (I-L), (I-M), or (I-N), wherein R₆ is NR′R″, NR′C(O)R″, NRC(O)NR′R″, NR′S(O)₂R″, C(O)NR′R″, or S(O)₂NR′R″, where R′ and R″ taken together with the nitrogen to which they are attached form a ring structure that optionally includes an additional heteroatom selected from N or O and is optionally substituted. In some embodiments, the R′ and R″ taken together with the nitrogen to which they are attached form a four-membered, five-membered, six-membered, seven-membered, eight-membered, or nine-membered ring structure that is optionally substituted.

In some embodiments, provided herein is a compound represented by any one of formulas (I) or (I-D), wherein R₃ is hydrogen; R₅ is hydrogen; and R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted. In certain specific embodiments, R₃ is hydrogen; R₅ is hydrogen; and R₂ is S(O)₂NR′R″; and R′ and R″ taken together with the nitrogen to which they are attached form a ring structure that optionally includes an additional heteroatom selected from N or O and is optionally substituted. In further or alternative embodiments, R₃ is hydrogen; R₅ is hydrogen; and R₂ hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, or SO₂NMe₂.

Provided herein in further or alternative embodiments, is a compound represented by any one of formulas (I) or (I-A), wherein R₂ is hydrogen; R₅ is hydrogen; and R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted. In further or alternative embodiments, R₂ is hydrogen; R₅ is hydrogen; and R₃ is hydroxy, CH₂F, CHF₂, CF₃, CH₂CH₂F, CH₂CHF₂, CH₂CF₃, methyl, ethyl, iso-propyl, cyclopropyl, cyclobutyl, cyclopentyl, methoxy, ethoxy, propoxy, iso-propoxy, OCF₃, C(O)NMe₂, or SO₂Me.

Provided herein in further or alternative embodiments, is a compound represented by any one of formulas (I) or (I-A), wherein R₂ is hydrogen; R₃ is hydrogen; and R₅ is hydroxy, halo, haloC₁-C₃alkyl, or C₁-C₃alkyl. In certain specific embodiments, R₂ is hydrogen; R₃ is hydrogen; and R₅ is hydroxy, F, or Cl.

In one embodiment, a compound of formula (I-Z-1) or formula (I-Z-2) is a compound of formula (I-D-1). In another aspect provided herein are compounds, or salt thereof, of formula (I-D-1):

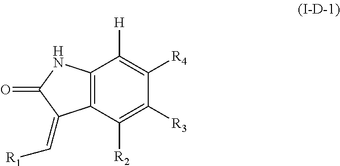

wherein
R₁ is pyrazole, imidazole, triazole, triazolone, indole, benzimidazole, azabenzimidazole, azaindole, benzothiazole or benzoxazole, where R₁ is optionally substituted with one, two, three, four, or five R₆;
   each R₆ is independently hydroxy, halo, haloalkyl, haloalkoxy, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
each of R₂, R₃, and R₄ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted;
   provided that at least one of R₂, R₃ and R₄ is not hydrogen; and
   further provided that when R₁ is indol-3-yl, R₃ is not Cl, and when R₁ is imidazo-2-yl, R₃ is not Cl;
where cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, cycloalkyl and heteroalicyclyl, and alkyl and alkoxy are independently, at each occurrence, optionally substituted with one or two groups selected from oxo, hydroxy, or amino.

In some embodiments of formula (I-D-1), R₁ is pyrazole, imidazole, triazole, triazolone, or benzimidazole, where R₁ is optionally substituted with one or two R₆. In some embodiments of formula (I-D-1), R₁ is pyrazole, triazole, triazolone, azaindole, benzimidazole, benzothiazole or benzoxazole, where R₁ is optionally substituted with one or two R₆. In some embodiments of formula (I-D-1), R₁ is imidazole, triazole, triazolone, or benzimidazole, where R₁ is optionally substituted with one or two R₆. In some embodiments of formula (I-D-1), R₁ is imidazole optionally substituted with one, two, or three R₆. In some embodiments of formula (I-D-1), R₁ is imidazole optionally substituted with one R₆. In some embodiments of formula (I-D-1), R₁ is imidazole optionally substituted with two R₆.

In some embodiments of formula (I-D-1), the imidazole has a structure of:

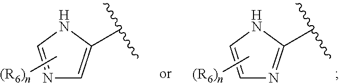

and
n is 0, 1, 2, or 3.

In some embodiments of formula (I-D-1), R₁ is pyrazole optionally substituted with one, two, or three R₆.

In some embodiments of formula (I-D-1), R₁ is pyrazole optionally substituted with one R₆. In some embodiments of formula (I-D-1), R₁ is pyrazole optionally substituted with two R₆.

In some embodiments of formula (I-D-1), the pyrazole has a structure of:

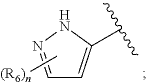

and
n is 0, 1, 2, or 3.

In some embodiments of formula (I-D-1), R₁ is triazole optionally substituted with one or two R₆. In some embodiments of formula (I-D-1), R₁ is triazole optionally substituted with one R₆. In some embodiments of formula (I-D-1), R₁ is triazole optionally substituted with two R₆.

In some embodiments of formula (I-D-1), the triazole has a structure of:

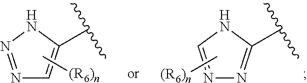

and
n is 0, 1, or 2.

In some embodiments of formula (I-D-1), R₁ is triazolone optionally substituted with one or two R₆. In some embodiments of formula (I-D-1), R₁ is triazolone optionally substituted with one R₆. In some embodiments of formula (I-D-1), R₁ is triazolone optionally substituted with two R₆.

In some embodiments, the triazolone has a structure of:

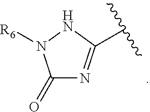

In some embodiments of formula (I-D-1), R₁ is benzimidazole optionally substituted with one, two, three, four or five R₆. In some embodiments of formula (I-D-1), R₁ is benzimidazole optionally substituted with one R₆. In some embodiments of formula (I-D-1), R₁ is benzimidazole optionally substituted with two R₆.

In some embodiments, the benzimidazole has a structure of:

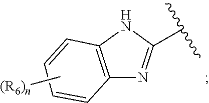

and
n is 0, 1, 2, 3, 4, or 5.

In some embodiments of formula (I-D-1), R₁ is indole optionally substituted with one, two, three, four or five R₆. In some embodiments of formula (I-D-1), R₁ is indole optionally substituted with one R₆. In some embodiments of formula (I-D-1), R₁ is indole optionally substituted with two R₆.

In some embodiments, the indole has a structure of:

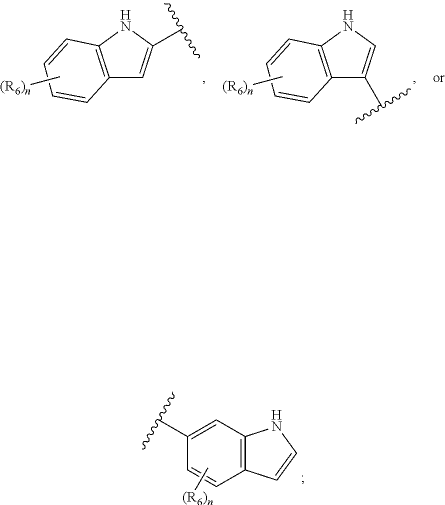

and
n is 0, 1, 2, 3, 4, or 5.

In some embodiments of formula (I-D-1), R₁ is azaindole optionally substituted with one, two, three, four or five R₆. In some embodiments of formula (I-D-1), R₁ is azaindole optionally substituted with one R₆. In some embodiments of formula (I-D-1), R₁ is azaindole optionally substituted with two R₆.

In some embodiments, the azaindole has a structure of:

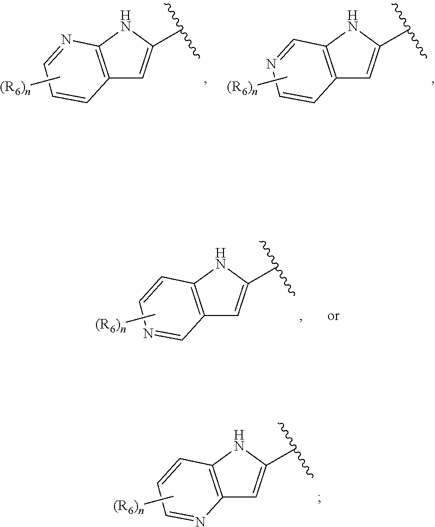

and
n is 0, 1, 2, 3, 4, or 5.

In some embodiments of compounds described above, R₆ is independently hydroxy, F, Cl, Br, I, CH₂F, CHF₂, CF₃, CH₂CH₂F, CH₂CHF₂, CH₂CF₃, methyl, ethyl, propyl, butyl, pentyl, hexyl, iso-propyl, cyclopropyl, cyclobutyl, cyclopentyl, cyclohexyl, methoxy, ethoxy, propoxy, iso-propoxy, phenyl, pyridyl, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, or SO₂NMe₂.

In some embodiments of compounds described above, R₆ is independently hydroxy, F, Cl, CF₃, CH₂CF₃, methyl, ethyl, propyl, butyl, iso-propyl, methoxy, ethoxy, propoxy, iso-propoxy, OCF₃, NHC(O)Me, C(O)NMe₂, C(O)NHMe, SO₂Me, or SO₂NMe₂. In some embodiments of compounds described above, R₆ is independently methyl, ethyl, butyl, SO₂Me, or C(O)NMe₂. In some embodiments of compounds described above, R₆ is SO₂Me, or C(O)NMe₂.

In some embodiments of compounds described above, R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of compounds described above, R₂ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments of compounds described above, R₂ is hydroxy, F, Cl, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, methoxy, ethoxy, iso-propoxy, OCF₃, C(O)NMe₂, or SO₂Me.

In some embodiments of compounds described herein, R₂ is S(O)₂NR′R″; and
R′ and R″ taken together with the nitrogen to which they are attached form a ring structure that optionally includes an additional heteroatom selected from N or O and is optionally substituted.

In some embodiments of compounds described above, R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of compounds described above, R₃ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, butoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments of compounds described above, R₃ is hydroxy, CH₂F, CHF₂, CF₃, CH₂CH₂F, CH₂CHF₂, CH₂CF₃, methyl, ethyl, methoxy, ethoxy, propoxy, iso-propyl, cyclopropyl, cyclobutyl, cyclopentyl, iso-propoxy, OCF₃, C(O)NMe₂, or SO₂Me. In some embodiments of compounds described above, R₃ is CF₃, CH₂CF₃, methyl, ethyl, iso-propyl, or cyclopropyl. In some embodiments of compounds described above, R₃ is methoxy, ethoxy, propoxy, iso-propoxy, or OCF₃. In some embodiments of compounds described above, R₃ is C(O)NMe₂ or SO₂Me.

In some embodiments of compounds described above, R₃ is C(O)NR′R″ or S(O)₂NR′R″; and
R′ and R″ taken together with the nitrogen to which they are attached form a ring structure that optionally includes an additional heteroatom selected from N or O and is optionally substituted.

In some embodiments of compounds described above, R₄ is hydroxy, halo, haloC₁-C₃alkyl, or C₁-C₃alkyl. In some embodiments of compounds described herein, R₄ is hydroxy, F, Cl, Br, I, CH₂F, CHF₂, CF₃, CH₂CH₂F, CH₂CHF₂, CH₂CF₃, methyl, ethyl, propyl, or iso-propyl. In some embodiments of compounds described herein, R₄ is fluoro. In some embodiments of compounds described herein, R₄ is chloro.

In some embodiments of compounds described above, R₃ is hydrogen or halogen; R₄ is halogen; and R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of compounds described above, R₃ is hydrogen; R₄ is fluoro or chloro; and R₂ is S(O)₂NR′R″; where R′ and R″ taken together with the nitrogen to which they are attached form a ring structure that optionally includes an additional heteroatom selected from N or O and is optionally substituted.

In some embodiments of compounds described above, R₃ is hydrogen; R₄ is fluoro or chloro; and R₂ hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, or SO₂NMe₂.

In some embodiments of compounds described above, R₂ is hydrogen or halogen; R₄ is halogen; and R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted.

In some embodiments of compounds described above, R₂ is hydrogen; R₄ is fluoro or chloro; and R₃ is hydroxy, CH₂F, CHF₂, CF₃, CH₂CH₂F, CH₂CHF₂, CH₂CF₃, methyl, ethyl, iso-propyl, cyclopropyl, cyclobutyl, cyclopentyl, methoxy, ethoxy, propoxy, iso-propoxy, OCF₃, C(O)NMe₂, or SO₂Me.

In one embodiment, a compound of formula (I-Z-1) or formula (I-Z-2) is a compound of formula (I-D-2). In another aspect, provided herein is a compound, or salt thereof, of formula (I-D-2):

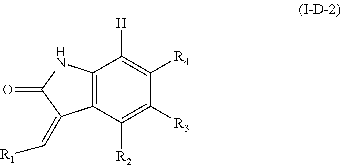

wherein
R₁ is pyrazolyl, imidazol-5-yl, triazolyl, triazolonyl, indoly-2-yl, indol-4-yl, indol-5-yl, indole-6-yl, indol-7-yl, benzimidazolyl, azabenzimidazolyl, azaindolyl, benzothiazolyl or benzoxazolyl, where R₁ is optionally substituted with one, two, three, four, or five R₆;
   each R₆ is independently hydroxy, halo, haloalkyl, haloalkoxy, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
each of R₂ and R₄ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₃ is hydroxy, halo, optionally substituted alkyl, cycloalkyl, optionally substituted alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted;
   where cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, and heteroalicyclyl, and alkyl and alkoxy are independently, at each occurrence, optionally substituted with one or two groups selected from oxo, hydroxy, or amino.

In some embodiments of formula (I-D-2) R₁ is pyrazolyl, imidazol-5-yl, triazolyl, triazolonyl, indoly-2-yl, indol-4-yl, indol-5-yl, indole-6-yl, indol-7-yl, benzimidazolyl, or azaindolyl where R₁ is optionally substituted with one or two R₆.

In some embodiments of formula (I-D-2) R₁ is imidazol-5-yl, triazolyl, triazolonyl, or benzimidazolyl, where R₁ is optionally substituted with one or two R₆.

In some embodiments of formula (I-D-2) R₁ is imidazol-5-yl, optionally substituted with one, two, or three R₆.

In some embodiments of formula (I-D-2) R₁ is imidazol-5-yl, optionally substituted with one R₆.

In some embodiments of formula (I-D-2) R₁ is imidazol-5-yl, optionally substituted with two R₆.

In some embodiments of formula (I-D-2) the imidazol-5-yl, has a structure of:

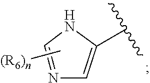

and
n is 0, 1, 2, or 3.

In some embodiments of formula (I-D-2) R₁ is pyrazolyl optionally substituted with one, two, or three R₆.

In some embodiments of formula (I-D-2) R₁ is pyrazolyl optionally substituted with one R₆.

In some embodiments of formula (I-D-2) R₁ is pyrazolyl optionally substituted with two R₆.

In some embodiments of formula (I-D-2) described above the pyrazole has a structure of:

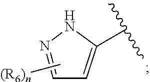

and
n is 0, 1, 2, or 3.

In some embodiments of formula (I-D-2) R₁ is triazolyl optionally substituted with one or two R₆.

In some embodiments of formula (I-D-2) R₁ is triazolyl optionally substituted with one R₆.

In some embodiments of formula (I-D-2) R₁ is triazolyl optionally substituted with two R₆.

In some embodiments of formula (I-D-2) described above, the triazole has a structure of:

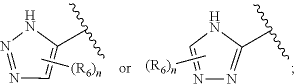

and
n is 0, 1, or 2.

In some embodiments of formula (I-D-2)R₁ is triazolonyl optionally substituted with one or two R₆.

In some embodiments of formula (I-D-2) R₁ is triazolonyl optionally substituted with one R₆.

In some embodiments of formula (I-D-2) R₁ is triazolonyl optionally substituted with two R₆.

In some embodiments of formula (I-D-2) described above, the triazolone has a structure of:

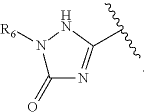

In some embodiments of formula (I-D-2) R₁ is benzimidazolyl optionally substituted with one, two, three, four or five R₆.

In some embodiments of formula (I-D-2) R₁ is benzimidazolyl optionally substituted with one R₆.

In some embodiments of formula (I-D-2) R₁ is benzimidazolyl optionally substituted with two R₆.

In some embodiments of formula (I-D-2) described above, the benzimidazole has a structure of

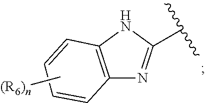

and
n is 0, 1, 2, 3, 4, or 5.

In some embodiments of formula (I-D-2) R₁ is indoly-2-yl, indol-4-yl, indol-5-yl, indole-6-yl, or indol-7-yl, optionally substituted with one, two, three, four or five R₆.

In some embodiments of formula (I-D-2) R₁ is indoly-2-yl, indol-4-yl, indol-5-yl, indole-6-yl, or indol-7-yl, optionally substituted with one R₆.

In some embodiments of formula (I-D-2) R₁ is indoly-2-yl, indol-4-yl, indol-5-yl, indole-6-yl, or indol-7-yl, optionally substituted with two R₆.

In some embodiments of formula (I-D-2) the indole has a structure of:

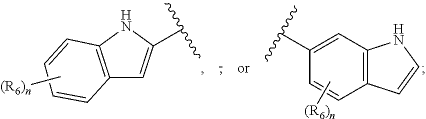

and
n is 0, 1, 2, 3, 4, or 5.

In some embodiments of formula (I-D-2) R₁ is azaindolyl optionally substituted with one, two, three, four or five R₆.

In some embodiments of formula (I-D-2) R₁ is azaindolyl optionally substituted with one R₆.

In some embodiments of formula (I-D-2) R₁ is azaindolyl optionally substituted with two R₆.

In some embodiments of formula (I-D-2) described above, the azaindole has a structure of:

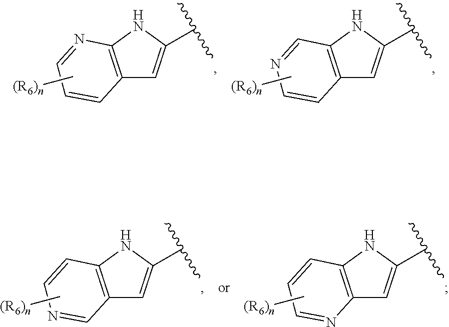

and
n is 0, 1, 2, 3, 4, or 5.

In some embodiments of formula (I-D-2) R₆ is independently hydroxy, F, Cl, Br, I, CH₂F, CHF₂, CF₃, CH₂CH₂F, CH₂CHF₂, CH₂CF₃, methyl, ethyl, propyl, butyl, pentyl, hexyl, iso-propyl, cyclopropyl, cyclobutyl, cyclopentyl, cyclohexyl, methoxy, ethoxy, propoxy, iso-propoxy, phenyl, pyridyl, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, or SO₂NMe₂.

In some embodiments of formula (I-D-2) R₆ is independently hydroxy, F, Cl, CF₃, CH₂CF₃, methyl, ethyl, propyl, butyl, iso-propyl, methoxy, ethoxy, propoxy, iso-propoxy, OCF₃, NHC(O)Me, C(O)NMe₂, C(O)NHMe, SO₂Me, or SO₂NMe₂.

In some embodiments of formula (I-D-2) R₆ is independently methyl, ethyl, butyl, SO₂Me, or C(O)NMe₂.

In some embodiments of formula (I-D-2) R₆ is SO₂Me, or C(O)NMe₂.

In some embodiments of formula (I-D-2) R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted;

In some embodiments of formula (I-D-2) R₂ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments of formula (I-D-2) R₂ is hydroxy, F, Cl, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, methoxy, ethoxy, iso-propoxy, OCF₃, C(O)NMe₂, or SO₂Me.

In some embodiments of formula (I-D-2) R₂ is S(O)₂NR′R″; and
R′ and R″ taken together with the nitrogen to which they are attached form a ring structure that optionally includes an additional heteroatom selected from N or O and is optionally substituted.

In some embodiments of formula (I-D-2) R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;

each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of formula (I-D-2) R₃ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, butoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments of formula (I-D-2) R₃ is hydroxy, CH₂F, CHF₂, CF₃, CH₂CH₂F, CH₂CHF₂, CH₂CF₃, methyl, ethyl, methoxy, ethoxy, propoxy, iso-propyl, cyclopropyl, cyclobutyl, cyclopentyl, iso-propoxy, OCF₃, C(O)NMe₂, or SO₂Me.

In some embodiments of formula (I-D-2) R₃ is CF₃, CH₂CF₃, methyl, ethyl, iso-propyl, or cyclopropyl.

In some embodiments of formula (I-D-2) R₃ is methoxy, ethoxy, propoxy, iso-propoxy, or OCF₃,

In some embodiments of formula (I-D-2) R₃ is C(O)NMe₂ or SO₂Me.

In some embodiments of formula (I-D-2) R₃ is C(O)NR′R″ or S(O)₂NR′R″; and
R′ and R″ taken together with the nitrogen to which they are attached form a ring structure that optionally includes an additional heteroatom selected from N or O and is optionally substituted.

In some embodiments of formula (I-D-2) R₄ is hydroxy, halo, haloC₁-C₃alkyl, or C₁-C₃alkyl.

In some embodiments of formula (I-D-2) R₄ is hydroxy, F, Cl, Br, I, CH₂F, CHF₂, CF₃, CH₂CH₂F, CH₂CHF₂, CH₂CF₃, methyl, ethyl, propyl, or iso-propyl.

In some embodiments of formula (I-D-2) R₄ is fluoro.

In some embodiments of formula (I-D-2) R₄ is chloro.

In some embodiments of formula (I-D-2) R₃ is hydrogen or halogen; R₄ is halogen; and R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of formula (I-D-2) R₃ is hydrogen; R₄ is fluoro or chloro; and R₂ is S(O)₂NR′R″; where R′ and R″ taken together with the nitrogen to which they are attached form a ring structure that optionally includes an additional heteroatom selected from N or O and is optionally substituted.

In some embodiments of formula (I-D-2) R₃ is hydrogen; R₄ is fluoro or chloro; and R₂ hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, or SO₂NMe₂.

In some embodiments of formula (I-D-2) R₂ is hydrogen or halogen; R₄ is halogen; and R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted.

In some embodiments of formula (I-D-2) R₂ is hydrogen; R₄ is fluoro or chloro; and R₃ is hydroxy, CH₂F, CHF₂, CF₃, CH₂CH₂F, CH₂CHF₂, CH₂CF₃, methyl, ethyl, iso-propyl, cyclopropyl, cyclobutyl, cyclopentyl, methoxy, ethoxy, propoxy, iso-propoxy, OCF₃, C(O)NMe₂, or SO₂Me.

In some embodiments the compound of formula (I-D-2) has the structure of formula (I-H-2):

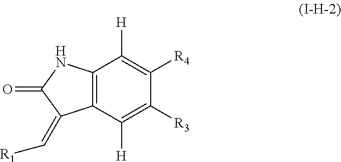

wherein
R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″, where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; and
R₄ is alkoxy, fluoro or chloro.

In some embodiments the compound of formula (I-D-2) has the structure formula (I-K-2):

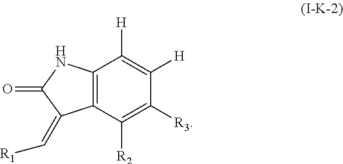

In some embodiments of formula (I-K-2) R₂ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments of formula (I-K-2) R₂ is hydroxy, F, Cl, CH₂F, CHF₂, CF₃, CH₂CH₂F, CH₂CHF₂, CH₂CF₃, methyl, ethyl, propyl, iso-propyl, cyclopropyl, methoxy, ethoxy, iso-propoxy, OCF₃, C(O)NMe₂, or SO₂Me.

In some embodiments of formula (I-K-2) R₂ is C(O)NMe₂ or SO₂Me.

In some embodiments of formula (I-K-2) R₂ is S(O)₂NR′R″; and
R′ and R″ taken together with the nitrogen to which they are attached form a ring structure that optionally includes an additional heteroatom selected from N or O and is optionally substituted.

In some embodiments the compound of formula (I-D-2) has the structure formula (I-L-2):

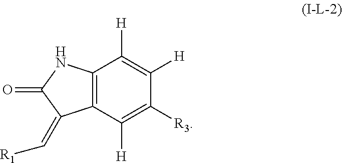

In some embodiments of formula (I-L-2) R₃ is hydroxy, halo, haloC₁-C₆alkyl, haloC₁-C₆alkoxy, C₁-C₆alkyl, C₃-C₆cycloalkyl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of formula (I-L-2) R₃ is CF₃, CH₂CF₃, methyl, ethyl, iso-propyl, or cyclopropyl.

In some embodiments of formula (I-L-2) R₃ is methoxy, ethoxy, propoxy, iso-propoxy, or OCF₃,

In some embodiments of formula (I-L-2) R₃ is C(O)NMe₂ or SO₂Me.

In some embodiments of formula (I-L-2) R₃ is C(O)NR′R″ or S(O)₂NR′R″; and
R′ and R″ taken together with the nitrogen to which they are attached form a ring structure that optionally includes an additional heteroatom selected from N or O and is optionally substituted.

Also provided herein are compounds of Formula I-1-2, or salt thereof:

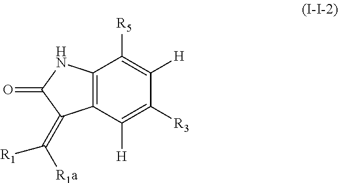

wherein
R₁ is pyrazole, imidazol-5-yl, triazolyl, triazolonyl, indoly-2-yl, indol-4-yl, indol-5-yl, indole-6-yl, indol-7-yl, benzimidazolyl, azabenzimidazolyl, azaindolyl, benzothiazolyl or benzoxazolyl, where R₁ is optionally substituted with one, two, three, four, or five R₆;
   each R₆ is independently hydroxy, halo, optionally substituted alkyl, optionally substituted alkoxy, optionally substituted cycloalkyl, optionally substituted aryl, optionally substituted heteroaryl, optionally substituted heteroalkyl optionally substituted heteroalicyclyl, optionally substituted alkylcycloalkyl, optionally substituted alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₁ₐ is hydrogen, optionally substituted alkyl, optionally substituted heteroalkyl, optionally substituted cycloalkyl or optionally substituted heteroalicyclyl;
each of R₂ and R₄ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₃ is hydrogen, hydroxy, halo, optionally substituted alkyl, optionally substituted cycloalkyl, optionally substituted alkoxy, optionally substituted cycloalkyloxy, optionally substituted aryl, optionally substituted heteroaryl, or optionally substituted heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₅ is hydrogen, halo, haloalkyl or alkyl; and
   each of R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted.

Also provided herein are compounds of formula (I-I-1) or salt thereof:

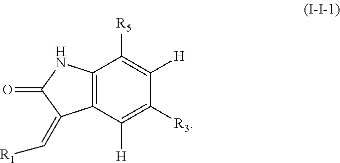

wherein
   R₁ is pyrazole, imidazol-5-yl, triazolyl, triazolonyl, indoly-2-yl, indol-4-yl, indol-5-yl, indole-6-yl, indol-7-yl, benzimidazolyl, azabenzimidazolyl, or azaindolyl, benzothiazolyl or benzoxazolyl, where R₁ is optionally substituted with one, two, three, four, or five R₆;
      each R₆ is independently hydroxy, halo, haloalkyl, haloalkoxy, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
   R₃ is hydrogen, hydroxy, halo, optionally substituted alkyl, optionally substituted cycloalkyl, optionally substituted alkoxy, optionally substituted cycloalkyloxy, optionally substituted aryl, optionally substituted heteroaryl, or optionally substituted heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
      each R, R′ and R″ are independently hydrogen, alkyl, haloalkyl, cycloalkyl, aryl, heteroaryl, or heteroalkyl; or
      R′ and R″ taken together with the nitrogen to which they are attached form a ring structure that optionally includes an additional heteroatom selected from N or O and is optionally substituted; and
   R₅ is hydroxy, halo, haloC₁-C₃alkyl, or C₁-C₃alkyl;
   where cycloalkyl, cycloalkyoxy, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, cycloalkyl and heteroalicyclyl, and alkyl and alkoxy are independently, at each occurrence, optionally substituted with one or two groups selected from oxo, hydroxy, or amino.

In some embodiments of formula (I-I-1), R₃ is hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of formula (I-I-1) R₅ is F or Cl.

In some embodiments of formula (I-I-1) R₁ is pyrazole, imidazol-5-yl, triazolyl, triazolonyl, indoly-2-yl, indol-4-yl, indol-5-yl, indole-6-yl, indol-7-yl, benzimidazolyl, azabenzimidazolyl, or azaindolyl, benzothiazolyl or benzoxazolyl where R₁ is optionally substituted with one or two R₆.

In some embodiments of formula (I-I-1) R₁ is imidazol-5-yl, triazolyl, triazolonyl, benzimidazolyl, or azabenzimidazolyl, where R₁ is optionally substituted with one or two R₆.

In some embodiments, the compound of any one of the formulas (I-D), (I-K), (I-L), (I-N), (I-D-2), (I-H-2), (I-K-2-1), (I-L-2) or (I-I-1) is radiolabeled.

Provided herein are pharmaceutical compositions comprising a compound of any one of formula (I) or Formula (I-A)-(I-N), (I-D-1), (I-D-2), (I-I-1) and (I-I-2) and a pharmaceutically acceptable carrier, excipient, or binder.

All of the embodiments for R₁, R₁ₐ, R₂, R₃, R₄, R₅ and R₆ described for compounds of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), are also applicable to compounds of formula (I-Z-1), (I-Z-2), (I-Z-2-a), (I-Z-2-b), (I-Z-2-c), (I-Z-2-d), (I-Z-3-a), (I-Z-3-b), (I-Z-3-c), and (I-Z-3-d) and vice versa.

Some embodiments provided herein describe a compound represented by any one of formulas (II), (I), (I-A), (I-B), (I-C), (I-D), (I-D-1), I-D-2), (I-E), (I-F), (I-G), (I-H), (I-I), (I-I-1), (I-I-2), (I-J), (I-K), (I-L), (I-M), (I-N), (I-I-1), (I-I-2), (I-Z-1), (I-Z-2), (I-Z-3-a-d), wherein

(A)—(1) R₃ is halo, alkoxy, haloalkoxy, or S(O)₂R; (2) R₃ is alkyl, haloalkyl, alkoxy or haloalkoxy; (3) R₃ is halo; (4) R₃ is alkoxy or cycloalkyloxy; and when R₃ is (1), (2), (3) or (4), R₂ is hydrogen, R₅ is hydrogen, and R₄ is hydrogen, halo, alkyl, haloalkyl, alkoxy or haloalkoxy; or when R₃ is (1), or (2), (3) or (4), R₂ is hydrogen, R₅ is hydrogen; and R₄ is halo, alkyl, haloalkyl, alkoxy or haloalkoxy;

or

(B)—(1) R₃ is halo, alkoxy, haloalkoxy, or S(O)₂R; (2) R₃ is alkyl, haloalkyl, alkoxy or haloalkoxy; (3) R₃ is halo; (4) R₃ is alkoxy or cycloalkyloxy; and when R₃ is (1), (2), (3) or (4), R₂ is hydrogen, R₄ is hydrogen, and R₅ is halo, alkyl, or haloalkyl; or, when R₃ is R₃ is (1), (2), (3) or (4), R₂ is hydrogen, R₄ is hydrogen, and R₅ is F or Cl.

Some additional embodiments provided herein describe a compound represented by any one of formulas (II), (I), (I-A), (I-B), (I-C), (I-D), (I-D-1), I-D-2), (I-E), (I-F), (I-G), (I-H), (I-I), (I-J), (I-K), (I-L), (I-M), (I-N) (I-I-1), (I-I-2), (I-Z-1), (I-Z-2), (I-Z-3-a-d), wherein, for any of (A) or (B) described above, R₁ is imidazol-5-yl optionally substituted by one or two R₆ selected from alkyl, haloalkyl, heteroalkyl, cycloalkyl, heteroalicyclyl, alkylcycloalkyl, or alkylheteroalicyclyl; or R₁ is pyrazole optionally substituted by one or two R₆ selected from alkyl, haloalkyl, heteroalkyl, cycloalkyl, heteroalicyclyl, alkylcycloalkyl, or alkylheteroalicyclyl; R₁ is imidazol-2-yl optionally substituted by one or two R₆ selected from alkyl, haloalkyl, heteroalkyl, cycloalkyl, heteroalicyclyl, alkylcycloalkyl, or alkylheteroalicyclyl; or R₁ is pyrazole optionally substituted by one or two R₆ selected from alkyl, haloalkyl, heteroalkyl, cycloalkyl, heteroalicyclyl, alkylcycloalkyl, or alkylheteroalicyclyl; or R₁ is benzimidazole optionally substituted by one or two R₆ selected from alkyl, haloalkyl, heteroalkyl, cycloalkyl, heteroalicyclyl, alkylcycloalkyl, or alkylheteroalicyclyl; where cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, and heteroalicyclyl.

Certain non-limiting examples of S(O)₂NR′R″; where R′ and R″ taken together with the nitrogen to which they are attached form a ring structure that optionally includes an additional heteroatom selected from N or O and is optionally substituted are set forth below:

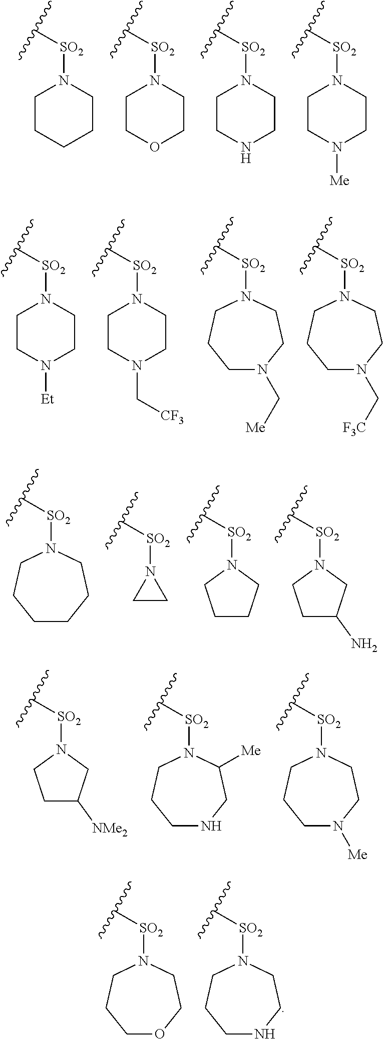

Certain non-limiting examples of NR′R″; where R′ and R″ taken together with the nitrogen to which they are attached form a ring structure that optionally includes an additional heteroatom selected from N or O and is optionally substituted are set forth below:

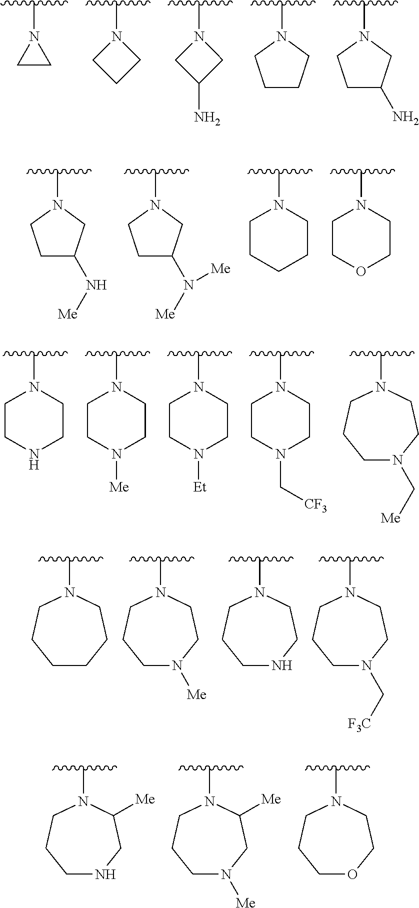

Certain non-limiting examples of C(O)NR′R″; where R′ and R″ taken together with the nitrogen to which they are attached form a ring structure that optionally includes an additional heteroatom selected from N or O and is optionally substituted are set forth below:

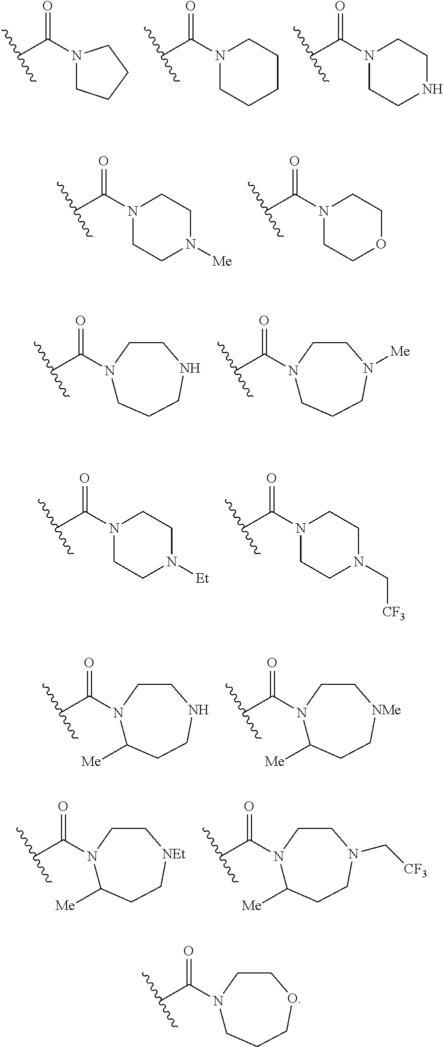

It will be understood that various compounds are possible within the scope of embodiments presented herein by combining embodiments of various variables and all such combinations are contemplated within the scope of embodiments provided herein. Any combination of the groups described above for the various variables is contemplated herein. Throughout the specification, groups and substituents thereof are chosen by one skilled in the field to provide stable moieties and compounds.

Non-limiting examples of compounds represented by any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) are set forth below:

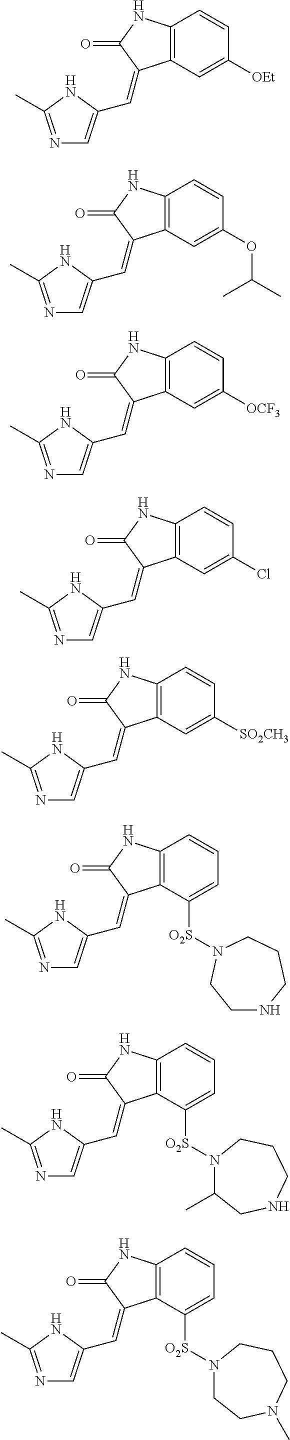

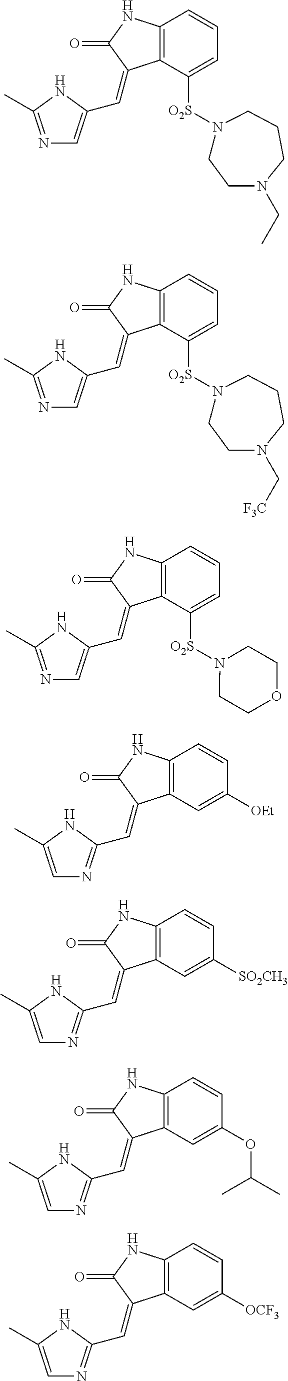

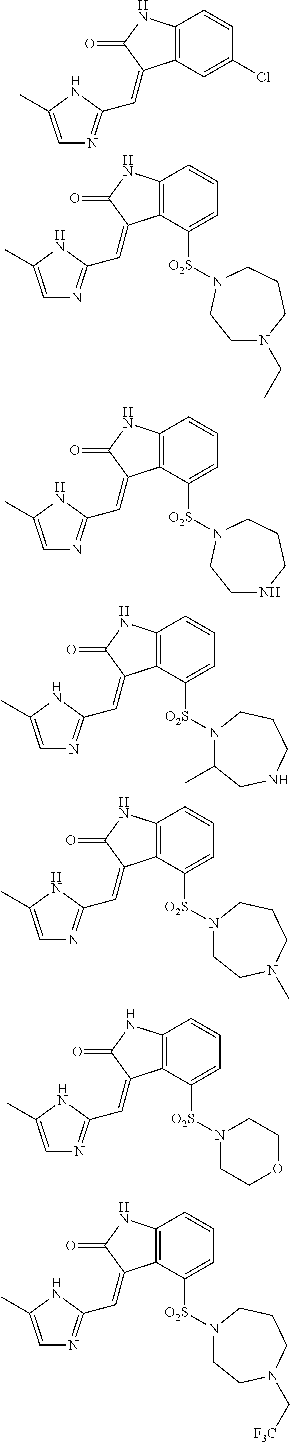

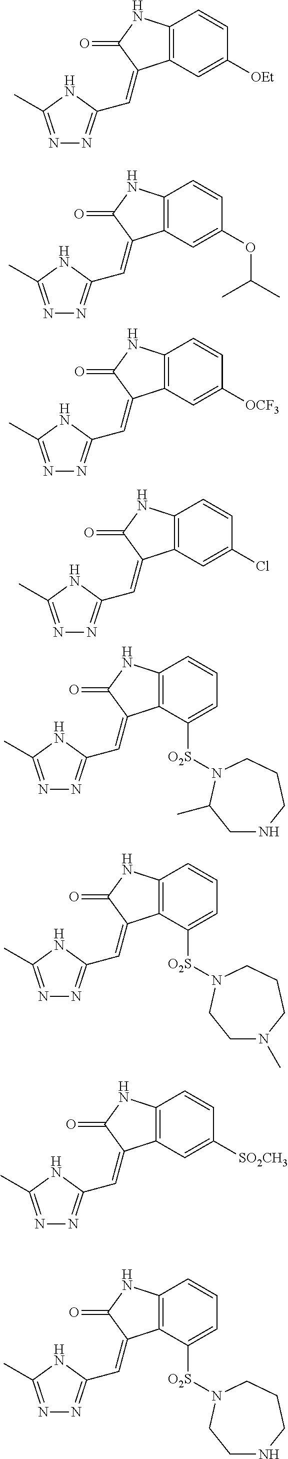

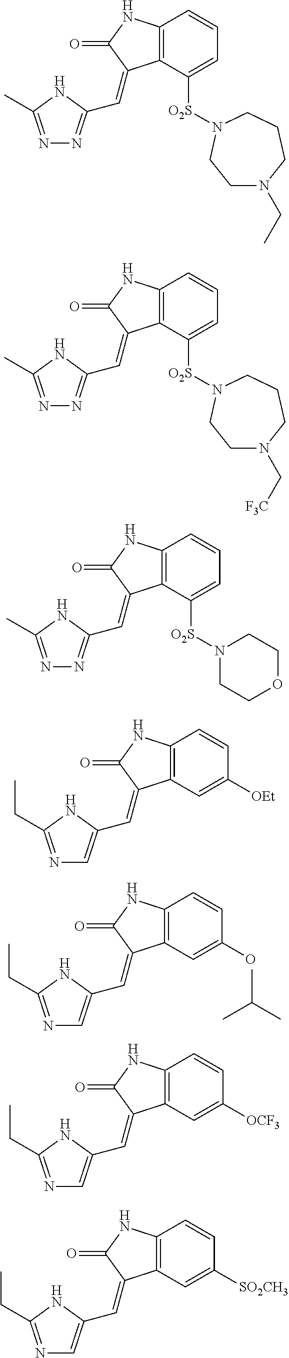

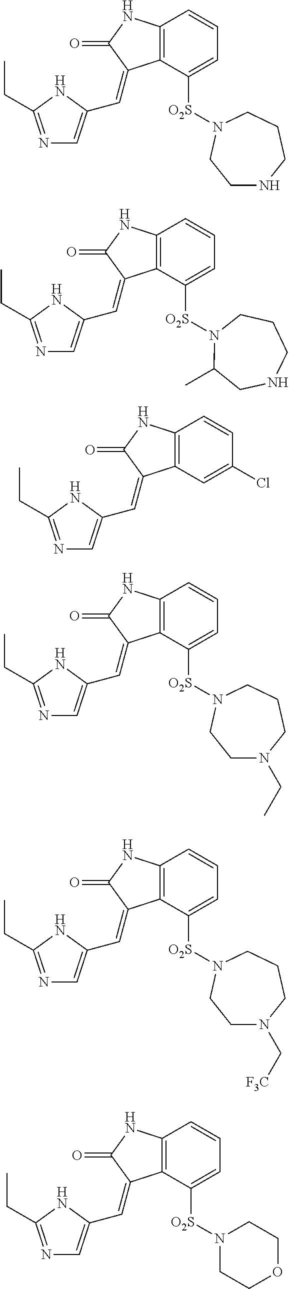

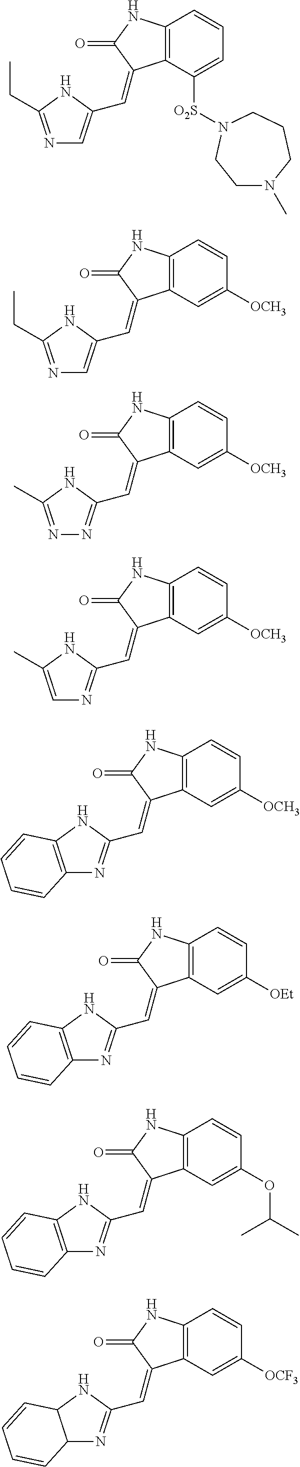

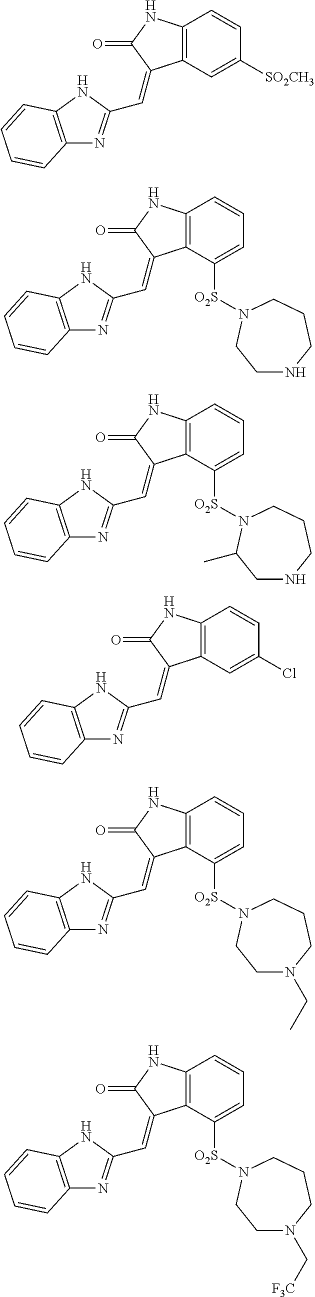

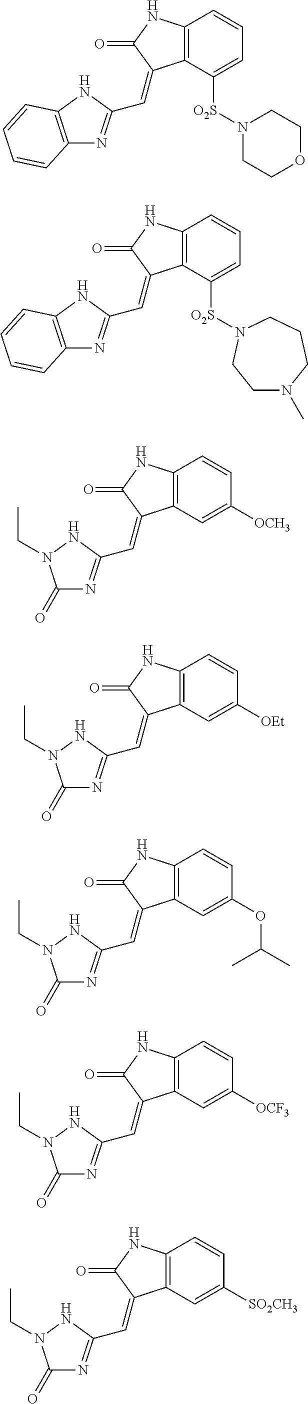

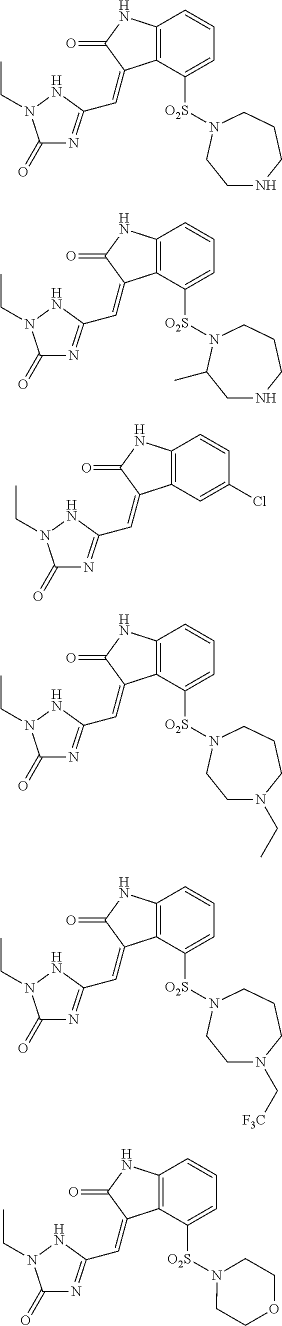

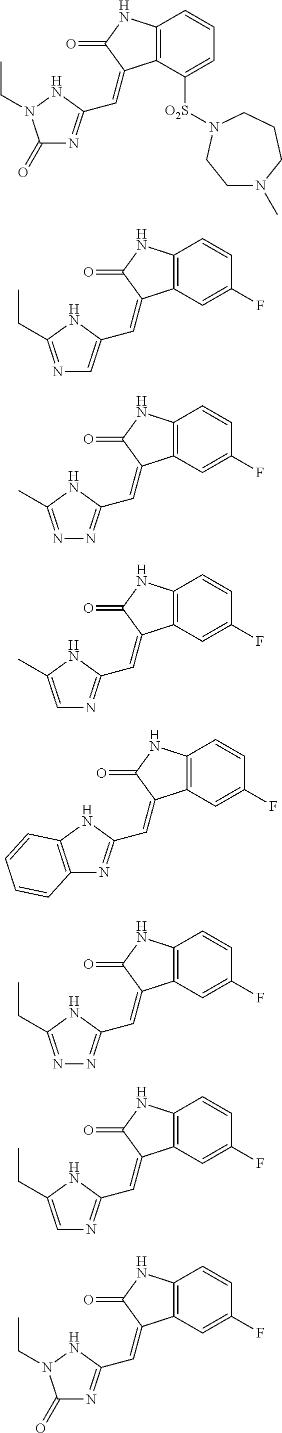

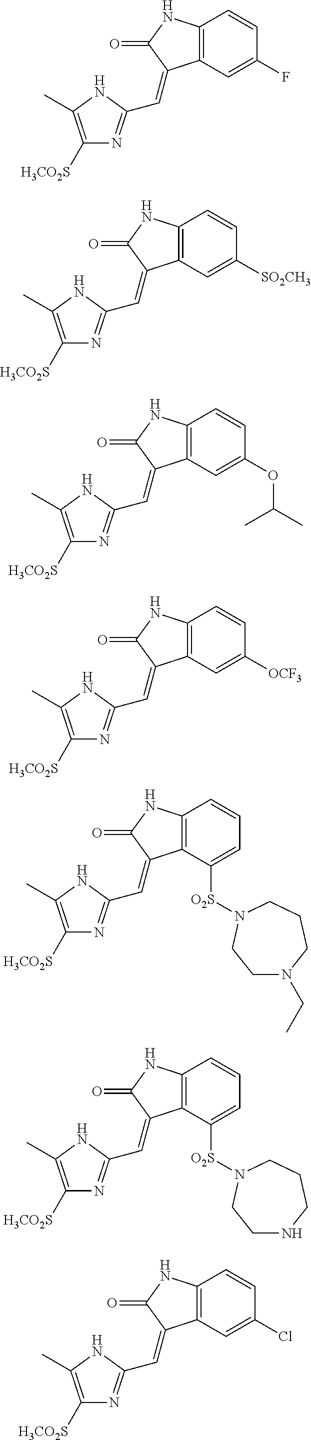

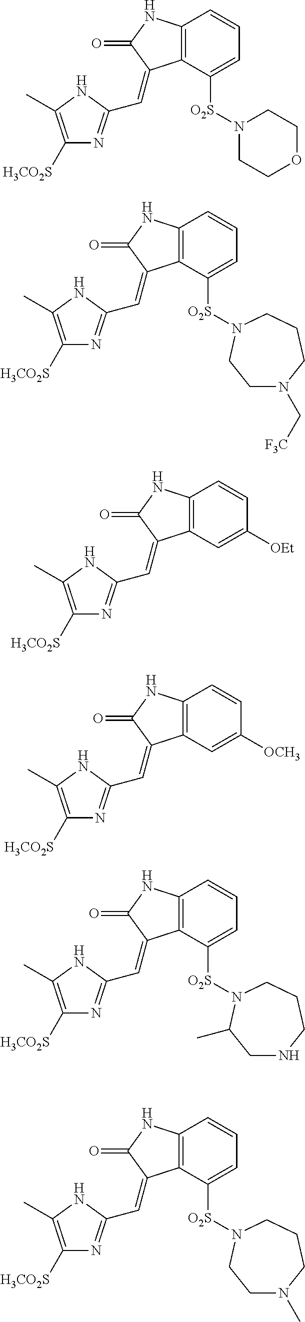

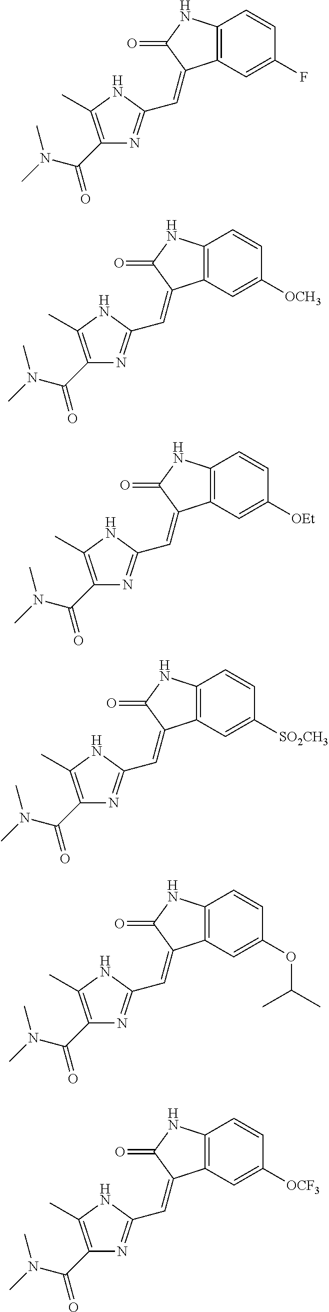

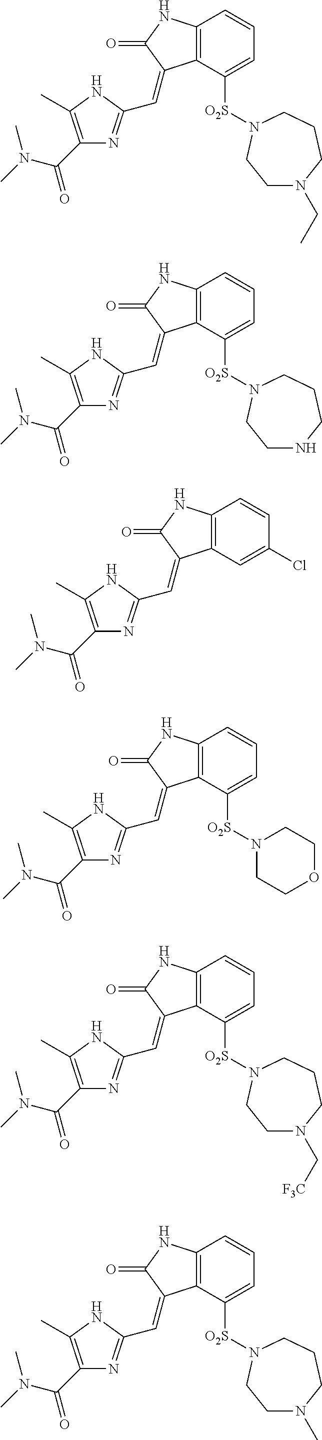 [SMILES: CCN1CCCN(S(=O)(=O)c2cccc3c2/C(=C/c2nc(C(=O)N(C)C)c(C)[nH]2)C(=O)N3)CC1 ; Cc1[nH]c(/C=C2\C(=O)Nc3cccc(S(=O)(=O)N4CCCNCC4)c32)nc1C(=O)N(C)C ; Cc1[nH]c(/C=C2\C(=O)Nc3ccc(Cl)cc32)nc1C(=O)N(C)C ; Cc1[nH]c(/C=C2\C(=O)Nc3cccc(S(=O)(=O)N4CCOCC4)c32)nc1C(=O)N(C)C ; Cc1[nH]c(/C=C2\C(=O)Nc3cccc(S(=O)(=O)N4CCCN(CC(F)(F)F)CC4)c32)nc1C(=O)N(C)C ; Cc1[nH]c(/C=C2\C(=O)Nc3cccc(S(=O)(=O)N4CCCN(C)CC4)c32)nc1C(=O)N(C)C]

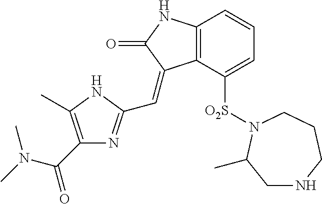 [SMILES: Cc1[nH]c(/C=C2\C(=O)Nc3cccc(S(=O)(=O)N4CCCNCC4C)c32)nc1C(=O)N(C)C]

Additional non-limiting examples of compounds represented by any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) are set forth in Example 1, Example 2, Table 1 and Table 2.

In a further aspect, provided herein are compounds, or salt thereof, of formula (I-Y):

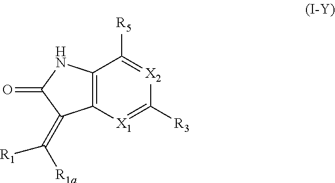

wherein
R₁ is pyrazole, imidazol-5-yl, triazolyl, triazolonyl, indoly-2-yl, indol-4-yl, indol-5-yl, indole-6-yl, indol-7-yl, benzimidazolyl, azabenzimidazolyl, azaindolyl, benzothiazolyl or benzoxazolyl, where R₁ is optionally substituted with one, two, three, four, or five R₆;
   each R₆ is independently hydroxy, halo, optionally substituted alkyl, optionally substituted alkoxy optionally substituted cycloalkyl, optionally substituted aryl, optionally substituted heteroaryl, optionally substituted heteroalkyl optionally substituted heteroalicyclyl, optionally substituted alkylcycloalkyl, optionally substituted alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₁ₐ is hydrogen, optionally substituted alkyl, optionally substituted cycloalkyl or optionally substituted heteroalicyclyl;
X₁ is N or C—R₂;
X₂ is N or C—R₄;
each of R₂ and R₄ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₃ is hydrogen, hydroxy, halo, optionally substituted alkyl, optionally substituted cycloalkyl, optionally substituted alkoxy, optionally substituted aryl, optionally substituted heteroaryl, or optionally substituted heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₅ is hydrogen, halo, haloalkyl or alkyl; and
   each of R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted;
   where alkyl, alkoxy, cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, and heteroalicyclyl.

In some embodiments of compounds of formula I-Y, X₁ is N and X₂ is C—R₄. In some embodiments of compounds of formula I-Y, X₂ is N and X₁ is C—R₂. All of the embodiments for R₁, R₁ₐ, R₆, R₂, R₃, R₄ and R₅ described previously for compounds of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), and (I-Z-3-d) also apply to compounds of formula I-Y.

In some embodiments, compounds of formula I-Y include the following compounds:

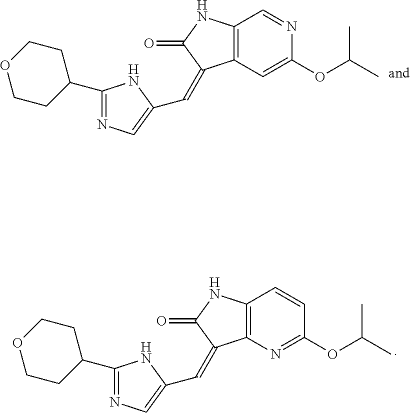 [SMILES: CC(C)Oc1cc2c(cn1)NC(=O)/C2=C\c1cnc(C2CCOCC2)[nH]1 ; CC(C)Oc1ccc2c(n1)/C(=C/c1cnc(C3CCOCC3)[nH]1)C(=O)N2]

Some embodiments provided herein describe a compound represented by any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) is radiolabeled. In some embodiments, the compounds described herein exist in their isotopically-labeled forms. In certain embodiments, the compound represented by any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) contains a tracer amount of radiolabeling.

In some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, sulfur, fluorine and chloride, such as ²H, ³H, ¹³C, ¹⁴C, ¹⁵N, ¹⁸0, ¹⁷O, ³¹P, ³²P, ³⁵S, ¹⁸F, and ³⁶Cl, respectively. In certain specific embodiments, the radiolabel is selected from tritium and carbon-14.

Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i. e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, a compound represented by any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) is detectably labeled. In further or additional embodiments, the compound represented by any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) has one or more ³H atoms (replacing ¹H atoms) incorporated into the compound. In other embodiments, any suitable detectable label is incorporated into the compound of formula compound represented by any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) (e.g., fluorophore conjugates, chemiluminescent conjugates, bioluminescent labels, and the like.) In certain embodiments, a isotopic variant of a compound represented by any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) is radiolabeled with one or more positron emitting isotopes such as carbon, oxygen, fluorine, chlorine, bromine, or iodine, including ¹⁰C, ¹¹C, ¹³O, ¹⁴O, ¹⁵O, ¹⁷F, ¹⁸F, ³²Cl, ³⁴Cl, ⁷⁴Br, ⁷⁵Br, ⁷⁶Br, ⁷⁷Br, ⁷⁸Br, ¹¹⁷I, ¹¹⁸I, ¹¹⁹I, ¹²⁰I, ¹²¹I, ¹²²I, ¹²⁴I, ¹²⁶I and ¹²⁸I.

Compounds of formula compound represented by any one of formulas (II), (I), (I-A), (I-B), (I-C), (I-D), (I-D-1), (I-D-2), (I-E), (I-F), (I-G), (I-H), (I-I), (I-I-1), (I-1-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) possess one or more stereocenters and each stereocenter exists independently in either the R or S configuration. In various embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis from optically-active starting materials, by chiral synthesis, or by chromatographic separation using a chiral stationary phase. In some embodiments, mixtures of one or more isomer is utilized as the therapeutic compound described herein. In certain embodiments, compounds described herein contains one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

The methods and formulations described herein include the use of N-oxides (if appropriate), crystalline forms (also known as polymorphs), solvates, amorphous phases, and/or pharmaceutically acceptable salts of compounds having the structure of any one of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d), as well as metabolites and active metabolites of these compounds having the same type of activity. In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p-tolunenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like. Solvates include water, ether (e.g., Tethrahydrofuran, methy tert-butyl ether) or alcohol (e.g., ethanol) solvates, acetates and the like. In specific embodiments, the compounds described herein exist in solvated forms with pharmaceutically acceptable solvents such as water, ethanol, and the like. In other embodiments, the compounds described herein exist in unsolvated form.

In some situations, compounds described herein may exist as tautomers. All tautomers are included within the scope of the compounds presented herein.

### Synthesis

Compounds of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) are synthesized according to procedures described in, for example, U.S. Pat. No. 6,846,839, and U.S. Pat. No. 5,886,020, and/or using commercially available starting materials. As used herein compounds of Formula (I) include compounds of formulas (I-A), (I-B), (I-C), (I-D), (I-D-1), (I-D-2), (I-E), (I-F), (I-G), (I-H), (I-I), (I-I-1), (I-J), (I-K), (I-L), (I-M), (I-N). Compounds of formula (I-Z-1) include compounds of formulas (I), (I-Z-2), (I-Z-2-a), (I-Z-2-b), (I-Z-2-c), (I-Z-2-d), (I-Z-3-a), (I-Z-3-b), (I-Z-3-c), (I-Z-3-d) and (I-I-2). Compounds of formula I-Y are synthesized using similar procedures.

In one embodiment, compounds of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) are prepared according to Scheme 1 described below.

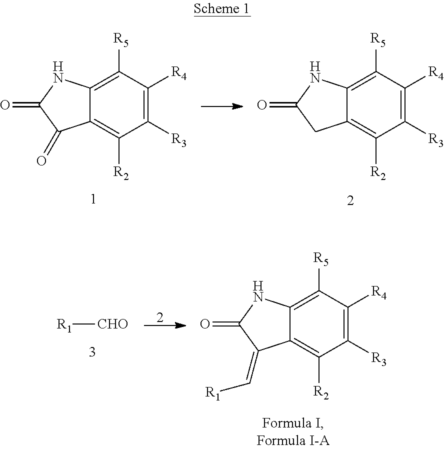

A substituted isatin 1—prepared, for example, by a Sandmeyer reaction, or from the corresponding indole by treating with a mix of InCl₃ and 2-iodoxybenzoic acid (IBX)—is reduced to an indolinone 2 by reduction such as, for example, a modified Wolff-Kishner reduction described by Crestini et al., Synthetic Communications: An International Journal for Rapid Communication of Synthetic Organic Chemistry, 1532-2432, Volume 24, Issue 20, 1994, pp 2835-2841. Coupling of a suitably protected aldehyde 3 with the compound 2 by heating in ethanol furnishes a compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d).

In another embodiment, compounds of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) are prepared according to the procedure described in Scheme 2 below.

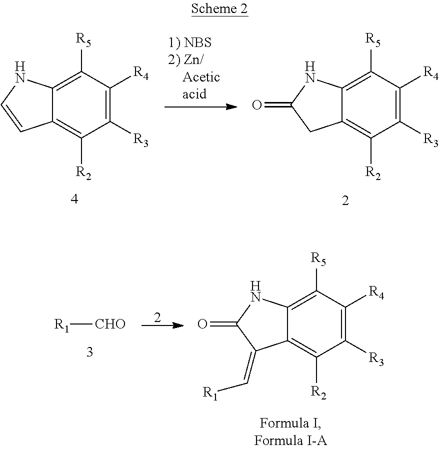

A substituted indole is oxidized in the presence of N-bromosuccinimide to the 2-indolinone compound 2. Coupling of a suitably protected aldehyde 3 with the compound 2 by heating in ethanol furnishes a compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d).

Scheme 3 below illustrates an exemplary synthesis of Synthesis of (Z)-3-((5-methyl-M-imidazol-2-yl)methylene)indolin-2-one

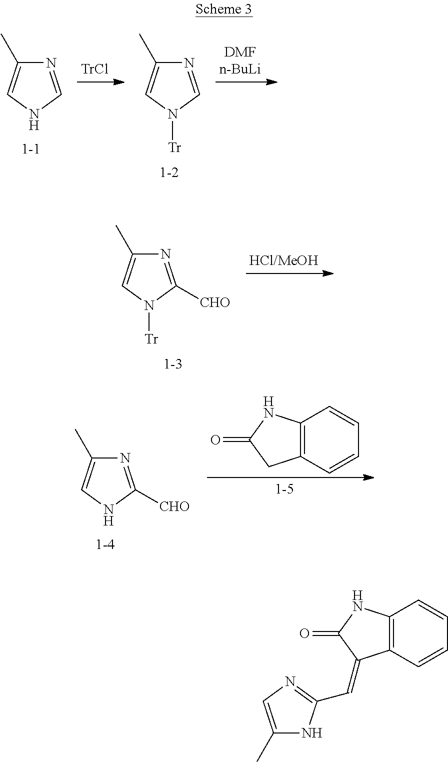

4-methyl-1H-imidazole is protected by reaction with Trityl chloride. Intermediate 1-2 is then deprotonated with n-BuLi, followed by addition of DMF. Reaction work up furnishes compound 1-3. The trityl protecting group is removed to furnish compound 1-4. The aldehyde 1-4 is condensed with compound 1-6 to provide the title compound.

Scheme 4 below illustrates an exemplary synthesis of (Z)-5-fluoro-3-((5-methyl-M-imidazol-2-yl)methylene)indolin-2-one

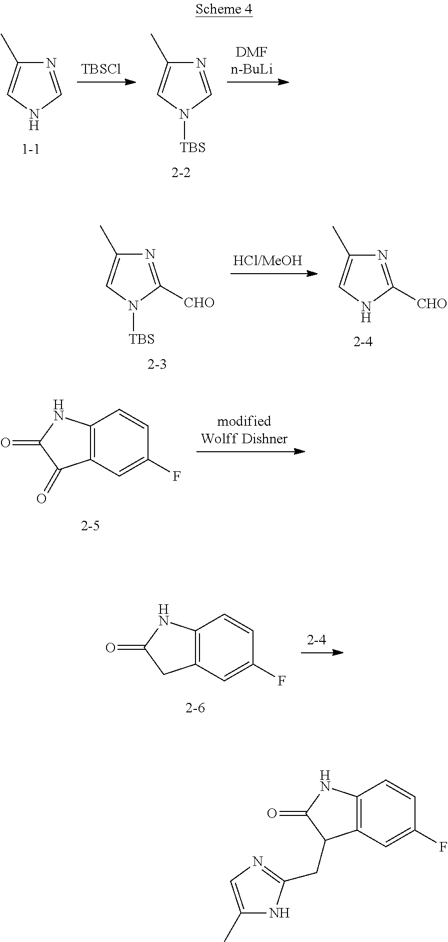

The aldehyde 2-4 is prepared using a procedure analogous to the procedure described above. Compound 2-5 is converted to compound 2-6 via a modified Wolff Kishner reduction. Compound 2-6 is then condensed with compound 2-4 to provide the title compound.

(Z)-5-methoxy-3-((5-methyl-M-imidazol-2-yl)methylene)indolin-2-one is prepared according to the procedure described in Scheme 4 above, starting with a suitably substituted isatin.

Scheme 5 illustrates an exemplary synthesis of (Z)-3-((1H-pyrrolo[2,3-b]pyridin-2-yl)methylene)indolin-2-one

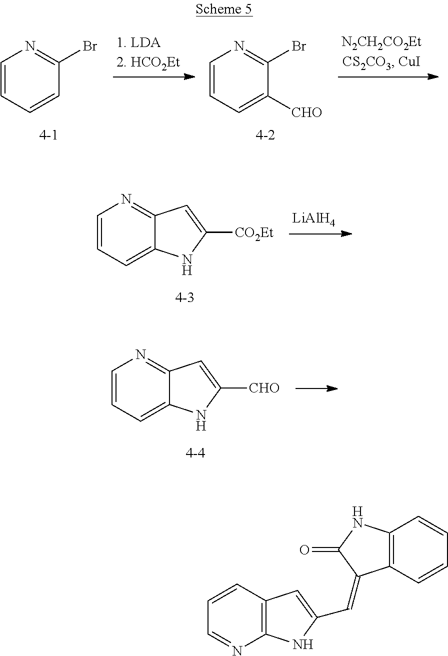 [SMILES: Brc1ccccn1 ; O=Cc1cccnc1Br ; CCOC(=O)c1cc2ncccc2[nH]1 ; "LiAlH4" ; O=Cc1cc2ncccc2[nH]1 ; O=C1Nc2ccccc2/C1=C/c1cc2cccnc2[nH]1]

This compound is prepared as shown above. Reaction of bromopyridine with ethyl formate in the presence of LDA furnishes the aldehyde 4-2, which upon reaction with ethyl 2-diazoacetate furnishes compound 4-3. Reduction of the ester group furnishes aldehyde 4-4 which on condensation with compound 1-6 provides the title compound.

### Formulations

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds of the invention and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization. The compounds described herein can be prepared as pharmaceutically acceptable salts formed when an acidic proton present in the parent compound either is replaced by a metal ion, for example an alkali metal ion, an alkaline earth ion, or an aluminum ion; or coordinates with an organic base. Base addition salts are be prepared by reacting the free acid form of the compounds described herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, the salt forms of the disclosed compounds can be prepared using salts of the starting materials or intermediates.

In some embodiments, the pharmaceutical compositions described herein contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use are optionally prepared according to known method, and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose, sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropylcellulose, or a time delay material such as ethyl cellulose, or cellulose acetate butyrate may be employed as appropriate. Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, for example glycerol, propylene glycol, sorbitol or sucrose. Such formulations may also contain a demulcent, a preservative, flavoring and coloring agents and antioxidant.

Pharmaceutical compositions may be in the form of a sterile injectable aqueous solution. Among the acceptable vehicles and solvents that may be employed are water, Ringer's solution and isotonic sodium chloride solution. The sterile injectable preparation may also be a sterile injectable oil-in-water microemulsion where the active ingredient is dissolved in the oily phase. For example, the active ingredient may be first dissolved in a mixture of soybean oil and lecithin. The oil solution then introduced into a water and glycerol mixture and processed to form a microemulsion. The injectable solutions or microemulsions may be introduced into an individual's blood-stream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump. The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

In certain embodiments, pharmaceutical compositions are administered in the form of suppositories for rectal administration of the drug. These compositions are prepared by mixing the active ingredient with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

In some embodiments, the compounds or compositions described herein are delivered in a vesicle, such as a liposome. In further or alternative embodiments, the compounds and pharmaceutical compositions described herein are delivered in a controlled release system, or a controlled release system can be placed in proximity of the therapeutic target. In one embodiment, a pump is used.

For topical use, creams, ointments, jellies, solutions or suspensions, etc., containing a compound described herein are used. As used herein, topical application includes mouth washes and gargles.

In certain embodiments, pharmaceutical compositions are administered in intranasal form via topical use of suitable intranasal vehicles and delivery devices, or via transdermal routes, using transdermal skin patches. To be administered in the form of a transdermal delivery system, the dosage administration will, of course, be continuous rather than intermittent throughout the dosage regimen.

In some embodiments, the formulations are conveniently presented in unit dosage form and are prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

### Methods

Provided in certain embodiments herein is a method of treating or preventing certain disorders or conditions. In some embodiments, the disorder or condition is a neurological disease or condition. In further or alternative embodiments, the disorder or condition is a neurodegenerative disease. In certain embodiments, neurological or neurodegenerative diseases or conditions include, for example, Alzheimer's disease, cerebral edema, cerebral ischemia, multiple sclerosis, neuropathies, Parkinson's disease, Huntington's disease, blunt or surgical trauma (including postsurgical cognitive dysfunction and spinal cord or brain stem injury), as well as the neurological aspects of disorders such as degenerative disc disease and sciatica.

Further examples of neurodegenerative disorders include, but are not limited to, Alexander's disease, Alper's disease, Alzheimer's disease, amyotrophic lateral sclerosis, ataxia telangiectasia, Batten disease, bovine spongiform encephalopathy, Canavan disease, Cockayne syndrome, corticobasal degeneration, Creutzfeldt-Jakob disease, Huntington's disease, HIV-associated dementia, Kennedy's disease, Krabbe's disease, lewy body dementia, Machado-Joseph disease, multiple sclerosis, multiple system atrophy, narcolepsy, neuroborreliosis, Parkinson's disease, Pelizaeus-Merzbacher Disease, Pick's disease, primary lateral sclerosis, prion diseases, Refsum's disease, Sandhoff s disease, Schilder's disease, subacute combined degeneration of spinal cord secondary to pernicious anaemia, schizophrenia, spinocerebellar ataxia, spinal muscular atrophy, Steele-Richardson-Olszewski disease, and tabes dorsalis.

In some embodiments, a neurodegenerative disease includes any pathological state involving neuronal degeneration, including Parkinson's Disease, Huntington's Disease, Alzheimer's Disease, and amyotrophic lateral sclerosis (ALS). Polyglutamine diseases, including Huntington's disease, are neurodegenerative diseases caused by an abnormally expanded polyglutamine tract in the causative gene products.

Provided herein in one aspect is a method of treating a neurodegenerative disease in a subject, the method comprising administering to the subject a therapeutically effective amount of a compound, or salt thereof, of Formula (I):

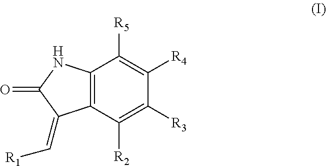

wherein
R₁ is a monocyclic or bicyclic heterocycle optionally substituted with one, two, three, four, or five R₆;
   each R₆ is independently hydroxy, halo, haloalkyl, haloalkoxy, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
each R₂, R₃, R₄, and R₅ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted.

Provided herein in another aspect is a method of treating a neurodegenerative disease in a subject, the method comprising administering to the subject a therapeutically effective amount of a compound, or salt thereof, of any one of formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d).

In some embodiments of the method, the neurodegenerative disease is Alexander's disease, Alper's disease, Alzheimer's disease, amyotrophic lateral sclerosis, ataxia telangiectasia, Batten disease, bovine spongiform encephalopathy, Canavan disease, Cockayne syndrome, corticobasal degeneration, Creutzfeldt-Jakob disease, Huntington's disease, HIV-associated dementia, Kennedy's disease, Krabbe's disease, lewy body dementia, Machado-Joseph disease, multiple sclerosis, multiple system atrophy, narcolepsy, neuroborreliosis, Parkinson's disease, Pelizaeus-Merzbacher Disease, Pick's disease, primary lateral sclerosis, prion diseases, Refsum's disease, Sandhoffs disease, Schilder's disease, subacute combined degeneration of spinal cord secondary to pernicious anaemia, schizophrenia, spinocerebellar ataxia, spinal muscular atrophy, Steele-Richardson-Olszewski disease, or tabes dorsalis.

In some embodiments of the method, the neurodegenerative disease is Parkinson's disease, Huntington's disease, Alzheimer's disease or amyotrophic lateral sclerosis. In some embodiments of the method, the subject is an individual suffering from or susceptible to a neurodegenerative disease. In some embodiments of the method, the individual is a human.

In another aspect, provided herein is a method for inhibiting a leucine-rich repeat kinase-2 (LRRK2) kinase, the method comprising contacting an LRRK2 kinase with a compound of formula (I):

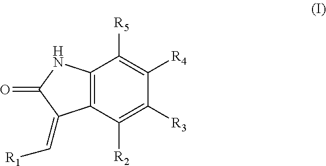

wherein
R₁ is a monocyclic or bicyclic heterocycle optionally substituted with one, two, three, four, or five R₆;
   each R₆ is independently hydroxy, halo, haloalkyl, haloalkoxy, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
each R₂, R₃, R₄, and R₅ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted;
   where cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, and heteroalicyclyl, and alkyl and alkoxy are independently, at each occurrence, optionally substituted with one or two groups selected from oxo, hydroxy, or amino.

Provided herein in another aspect is a method for inhibiting a leucine-rich repeat kinase-2 (LRRK2) kinase, the method comprising contacting an LRRK2 kinase with a compound, or salt thereof, of any one of formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d).

In another aspect provided herein is a method for treating a disorder or condition that is treated by inhibiting LRRK2 activity in a subject in need of treatment thereof, the method comprising administering to the subject a therapeutically effective amount of a compound of formula (I):

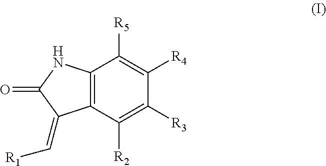

wherein
R₁ is a monocyclic or bicyclic heterocycle optionally substituted with one, two, three, four, or five R₆;
   each R₆ is independently hydroxy, halo, haloalkyl, haloalkoxy, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
each R₂, R₃, R₄, and R₅ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted;
   where cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, and heteroalicyclyl, and alkyl and alkoxy are independently, at each occurrence, optionally substituted with one or two groups selected from oxo, hydroxy, or amino.

Provided herein in another aspect is a method for treating a disorder or condition that is treated by inhibiting LRRK2 activity in a subject in need of treatment thereof, the method comprising administering to the subject a therapeutically effective amount of a compound, or salt thereof, of any one of formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d).

In a further aspect provided herein is a method of treating or preventing nerve cell degeneration, the method comprising administering to a subject suffering from or susceptible to nerve cell degeneration a therapeutically effective amount of a compound, or salt thereof, of Formula (I):

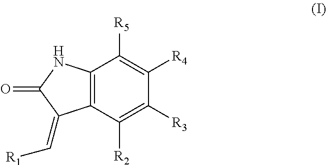

wherein
R₁ is a monocyclic or bicyclic heterocycle optionally substituted with one, two, three, four, or five R₆;
   each R₆ is independently hydroxy, halo, haloalkyl, haloalkoxy, alkyl, cycloalkyl, alkoxy, aryl, heteroaryl, heteroalkyl, heteroalicyclyl, alkylcycloalkyl, or alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
each R₂, R₃, R₄, and R₅ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″; and
   each R, R′ and R″ are independently hydrogen, alkyl, haloalkyl, cycloalkyl, aryl, heteroaryl, or heteroalkyl; or
   R′ and R″ taken together with the nitrogen to which they are attached form a ring structure that optionally includes an additional heteroatom selected from N or O and is optionally substituted;
where cycloalkyl, aryl, heteroaryl and heteroalicyclyl are independently, at each occurrence, optionally substituted with one or two groups selected from oxo, hydroxy, amino, cyano, halo, alkyl, haloalkyl, alkoxy, haloalkoxy, aminoalkyl, aminodialkyl, heteroalkyl, S(O)₂R, NR′C(O)R″, C(O)NR′R″, and heteroalicyclyl, and alkyl and alkoxy are independently, at each occurrence, optionally substituted with one or two groups selected from oxo, hydroxy, or amino.

Provided herein in another aspect is a method of treating or preventing nerve cell degeneration, the method comprising administering to a subject suffering from or susceptible to nerve cell degeneration a therapeutically effective amount of a compound, or salt thereof, of any one of formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d).

Genetic studies have also implicated LRRK2 in the pathogenesis of cancers, Crohn's disease and leprosy. Provided herein in another aspect is a method of treating Crohn's disease comprising administering to a subject suffering from or susceptible to nerve cell degeneration a therapeutically effective amount of a compound, or salt thereof, of any one of formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d). In an additional aspect, provided herein is a method of treating leprosy comprising administering to a subject suffering from or susceptible to nerve cell degeneration a therapeutically effective amount of a compound, or salt thereof, of any one of formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d). In yet another aspect, provided herein is a method of treating cancer comprising administering to a subject suffering from or susceptible to nerve cell degeneration a therapeutically effective amount of a compound, or salt thereof, of any one of formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d). In some of such embodiments, the cancer is melanoma. In some other embodiments, the cancer is a non-skin cancer which occurs concomitant with Parkinson's disease.

In some embodiments of the methods described above, R₁ is pyrazole, pyrrole, imidazole, triazole, triazolone, indole, benzimidazole, azabenzimidazole, azaindole, benzothiazole or benzoxazole, where R₁ is optionally substituted with one, two, three, four or five R₆.

In some embodiments of the methods described above, R₁ is pyrazole, imidazole, triazole, triazolone, or benzimidazole, where R₁ is optionally substituted with one or two R₆.

In some embodiments of the methods described above, R₁ is imidazole, triazole, triazolone, or benzimidazole, benzothiazole or benzoxazole, where R₁ is optionally substituted with one or two R₆.

In some embodiments of the methods described above, R₁ is imidazole optionally substituted with one, two, or three R₆.

In some embodiments of the methods described above R₁ is imidazole optionally substituted with one R₆.

In some embodiments of the methods described above R₁ is imidazole optionally substituted with two R₆.

In some embodiments of the methods described above the imidazole has a structure of:

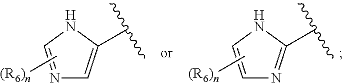

and
n is 0, 1, 2, or 3.

In some embodiments of the methods described above R₁ is pyrazole optionally substituted with one, two, or three R₆.

In some embodiments of the methods described above R₁ is pyrazole optionally substituted with one R₆.

In some embodiments of the methods described above R₁ is pyrazole optionally substituted with two R₆.

In some embodiments of the methods described above, the pyrazole has a structure of:

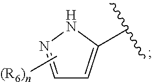

and
n is 0, 1, 2, or 3.

In some embodiments of the methods described above, R₁ is pyrrole optionally substituted with one, two, three, or four R₆.

In some embodiments of the methods described above, R₁ is pyrrole optionally substituted with one R₆.

In some embodiments of the methods described above, R₁ is pyrrole optionally substituted with two R₆.

In some embodiments of the methods described above, the pyrrole has a structure of:

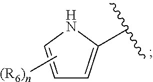

and
n is 0, 1, 2, 3, or 4.

In some embodiments of the methods described above, R₁ is triazole optionally substituted with one or two R₆.

In some embodiments of the methods described above, R₁ is triazole optionally substituted with one R₆.

In some embodiments of the methods described above, R₁ is triazole optionally substituted with two R₆.

In some embodiments of the methods described above, the triazole has a structure of:

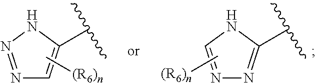

and
n is 0, 1, or 2.

In some embodiments of the methods described above, R₁ is triazolone optionally substituted with one or two R₆. In some embodiments of the methods described above, R₁ is triazolone optionally substituted with one R₆. In some embodiments of the methods described above, R₁ is triazolone optionally substituted with two R₆.

In some embodiments of the methods described above, the triazolone has a structure of:

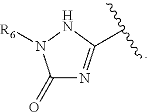

In some embodiments of the methods described above, R₁ is benzimidazole optionally substituted with one, two, three, four or five R₆. In some embodiments of the methods described above, R₁ is benzimidazole optionally substituted with one R₆. In some embodiments of the methods described above, R₁ is benzimidazole optionally substituted with two R₆.

In some embodiments of the methods described above, the benzimidazole has a structure of:

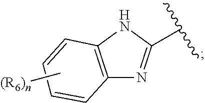

and
n is 0, 1, 2, 3, 4, or 5.

In some embodiments of the methods described above, R₁ is indole optionally substituted with one, two, three, four or five R₆. In some embodiments of the methods described above, R₁ is indole optionally substituted with one R₆. In some embodiments of the methods described above, R₁ is indole optionally substituted with two R₆.

In some embodiments of the methods described above, the indole has a structure of:

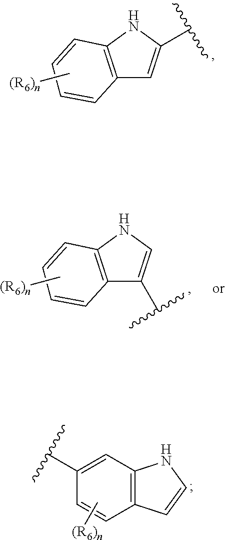

and
n is 0, 1, 2, 3, 4, or 5.

In some embodiments of the methods described above, R₁ is azaindole optionally substituted with one, two, three, four or five R₆. In some embodiments of the methods described above, R₁ is azaindole optionally substituted with one R₆. In some embodiments of the methods described above, R₁ is azaindole optionally substituted with two R₆.

In some embodiments of the methods described above, the azaindole has a structure of:

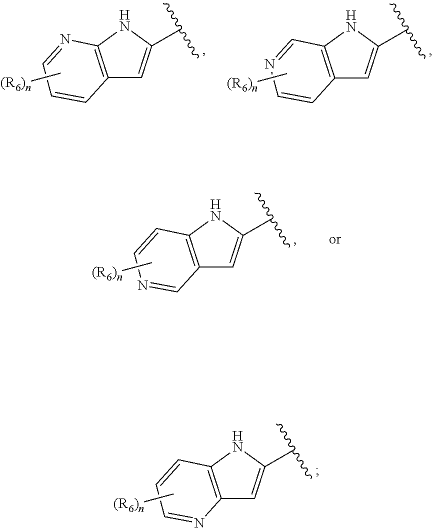

and
n is 0, 1, 2, 3, 4, or 5.

In some embodiments of the methods described above, R₆ is independently hydroxy, F, Cl, Br, I, CH₂F, CHF₂, CF₃, CH₂CH₂F, CH₂CHF₂, CH₂CF₃, methyl, ethyl, propyl, iso-propyl, butyl, sec-butyl, iso-butyl, tert-butyl, pentyl, hexyl, cyclopropyl, cyclobutyl, cyclopentyl, cyclohexyl, methoxy, ethoxy, propoxy, iso-propoxy, butoxy, phenyl, pyridyl, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, or SO₂NMe₂.

In some embodiments of the methods described above, R₆ is independently methyl, ethyl, propyl, iso-propyl, butyl, phenyl, SO₂Me, or C(O)NMe₂.

In some embodiments of the methods described above, the compound of formula (I) has a structure of any of formulas (I-A), (I-D), (I-E), (I-F), (I-G), or (I-H):

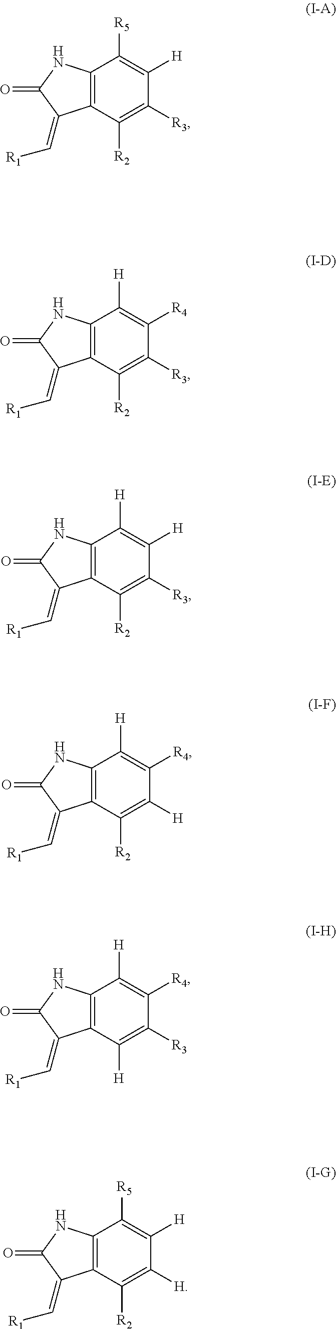

In some embodiments of the methods described above, R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of the methods described above, R₂ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments of the methods described above, R₂ is hydroxy, F, Cl, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, methoxy, ethoxy, iso-propoxy, OCF₃, C(O)NMe₂, or SO₂Me.

In some embodiments of the methods described above, R₂ is S(O)₂NR′R″; and
R′ and R″ taken together with the nitrogen to which they are attached form a ring structure that optionally includes an additional heteroatom selected from N or O and is optionally substituted.

In some embodiments of the methods described above, R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of the methods described above, R₃ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments of the methods described above, R₃ is hydroxy, CH₂F, CHF₂, CF₃, CH₂CH₂F, CH₂CHF₂, CH₂CF₃, methyl, ethyl, iso-propyl, cyclopropyl, cyclobutyl, cyclopentyl, methoxy, ethoxy, propoxy, iso-propoxy, OCF₃, C(O)NMe₂, or SO₂Me. In some embodiments of the methods described above, R₃ is CF₃, CH₂CF₃, methyl, ethyl, iso-propyl, cyclopropyl, methoxy, ethoxy, propoxy, iso-propoxy, OCF₃, C(O)NMe₂, or SO₂Me.

In some embodiments of the methods described above, R₃ is C(O)NR′R″ or S(O)₂NR′R″; and
R′ and R″ taken together with the nitrogen to which they are attached form a ring structure that optionally includes an additional heteroatom selected from N or O and is optionally substituted.

In some embodiments of the methods described above, R₄ is hydroxy, halo, haloC₁-C₃alkyl, or C₁-C₃alkyl.

In some embodiments of the methods described above, R₄ is F or Cl. In some embodiments of the methods described above, R₅ is hydroxy, F, Cl, CF₃, or methyl. In some embodiments of the methods described above, R₅ is F or Cl.

In some embodiments of the methods described above, the compound of formula (I) has the structure of formula (I-D), wherein R₃ is hydrogen or halogen; R₄ is halogen; and R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of the methods described above, the compound of formula (I) has the structure of formula (I-D), wherein R₃ is hydrogen; R₄ is fluoro or chloro; and R₂ is S(O)₂NR′R″; and
R′ and R″ taken together with the nitrogen to which they are attached form a ring structure that optionally includes an additional heteroatom selected from N or O and is optionally substituted.

In some embodiments of the methods described above, the compound of formula (I) has the structure of formula (I-D), wherein R₃ is hydrogen; R₄ is fluoro or chloro; and R₂ hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, or SO₂NMe₂.

In some embodiments of the methods described above, the compound of formula (I) has the structure of formula (I-D), wherein R₂ is hydrogen or halogen; R₄ is halogen; and R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of the methods described above, the compound of formula (I) has the structure of formula (I-D), wherein R₂ is hydrogen; R₄ is fluoro or chloro; and R₃ is hydroxy, CH₂F, CHF₂, CF₃, CH₂CH₂F, CH₂CHF₂, CH₂CF₃, methyl, ethyl, iso-propyl, cyclopropyl, cyclobutyl, cyclopentyl, methoxy, ethoxy, propoxy, iso-propoxy, OCF₃, C(O)NMe₂, or SO₂Me.

In some embodiments of the methods described above, the compound of formula (I) has the structure of formula (I-F), wherein R₂ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″; where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; and R₄ is fluoro or chloro.

In some embodiments of the methods described above, the compound of formula (I) has the structure of formula (I-H), wherein R₃ is hydroxy, halo, haloC₁-C₆alkyl, haloC₁-C₆alkoxy, C₁-C₆alkyl, heteroC₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″, where each R, R′ and R″ are independently hydrogen, alkyl, haloalkyl, cycloalkyl, aryl, heteroaryl, or heteroalkyl; or R′ and R″ taken together with the nitrogen to which they are attached form a ring structure that optionally includes an additional heteroatom selected from N or O and is optionally substituted; and R₄ is fluoro or chloro.

In some embodiments of the methods described above, the compound of formula (I) has a structure formula (I-K):

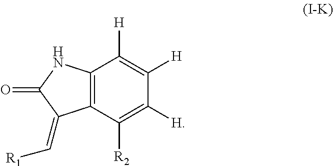

In some embodiments of the methods described above, R₂ is hydroxy, F, Cl, Br, I, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, cyclohexyl, methoxy, ethoxy, propoxy, iso-propoxy, OCH₂F, OCHF₂, OCF₃, CO₂Me, CO₂Et, CO₂H, NHC(O)Me, C(O)NMe₂, C(O)NH₂, C(O)NHMe, SO₂Me, SO₂Et, SO₂NMe₂, C(O)NR′R″, or S(O)₂NR′R″; and
R′ and R″ taken together with the nitrogen to which they are attached form a ring structure that optionally includes an additional heteroatom selected from N or O and is optionally substituted.

In some embodiments of the methods described above, R₂ is hydroxy, F, Cl, CH₂F, CHF₂, CF₃, CH₂CH₂F, CH₂CHF₂, CH₂CF₃, methyl, ethyl, propyl, iso-propyl, cyclopropyl, methoxy, ethoxy, iso-propoxy, OCF₃, C(O)NMe₂, or SO₂Me.

In some embodiments of the methods described above, R₂ is C(O)NMe₂ or SO₂Me.

In some embodiments of the methods described above, R₂ is S(O)₂NR′R″; and
R′ and R″ taken together with the nitrogen to which they are attached form a ring structure that optionally includes an additional heteroatom selected from N or O and is optionally substituted.

In some embodiments of the methods described above, the compound of formula (I) has a structure of formula (I-H):

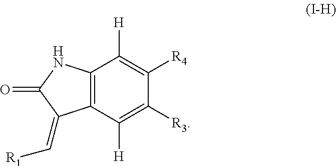

In some embodiments of the methods described above, the compound of formula (I) has a structure formula (I-L):

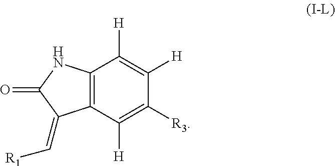

In some embodiments of the methods described above, R₃ is hydroxy, halo, haloC₁-C₆alkyl, haloC₁-C₆alkoxy, C₁-C₆alkyl, C₃-C₆cycloalkyl, C₁-C₆alkoxy, NR′R″, NR′C(O)R″, NRC(O)NR′R″, NR′S(O)₂R″, C(O)NR′R″, S(O)₂R, or S(O)₂NR′R″;
each R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

In some embodiments of the methods described above, R₃ is CF₃, CH₂CF₃, methyl, ethyl, iso-propyl, or cyclopropyl. In some embodiments of the methods described above, R₃ is methoxy, ethoxy, propoxy, iso-propoxy, or OCF₃. In some embodiments of the methods described above, R₃ is C(O)NMe₂ or SO₂Me.

In some embodiments of the methods described above, R₃ is C(O)NR′R″ or S(O)₂NR′R″; and
R′ and R″ taken together with the nitrogen to which they are attached form a ring structure that optionally includes an additional heteroatom selected from N or O and is optionally substituted.

In some embodiments of the methods described above, R₄ is hydrogen, hydroxyl, F, Cl, methoxy, ethoxy, propoxy, iso-propoxy, or OCF₃.

In some embodiments of the methods described above, the compound of formula (I) has the structure formula (I-I):

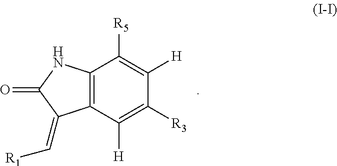

In some embodiments of the methods described above, the compound of formula (I) has the structure formula (I-N):

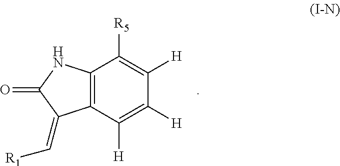

In particular embodiments, the disorder or condition comprises Parkinson's disease or a Parkinson-plus syndrome. Parkinson-plus syndromes include multiple system atropy (MSA) and progressive supranuclear party (PSP). In certain embodiments, the compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) is used to treat Parkinson's disease that presents in several forms, including, but not limited to sporadic Parkinson's disease, a familial form of Parkinson's disease, autosomal recessive early-onset Parkinson's disease, or post-encephalitic Parkinson's disease. In some embodiments, a therapeutically effective amount of a compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) which, when administered to a subject having Parkinson's disease, or a Parkinson-plus syndrome, ameliorates or lessens the severity of one or more of the symptoms of the disease. In certain specific embodiments, the symptoms of Parkinson's disease include but are not limited to tremor, rigidity of the limbs and trunk, akinesia, bradykinesia and postural abnormalities.

In another aspect, provided herein in certain embodiments, is a method for treating a neurological or neurodegenerative disease or condition by administering to a subject a therapeutically effective amount of the compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) or a pharmaceutically acceptable salt thereof. Also provided herein are methods to treat, prevent, delay the onset or progression of, or alleviate symptoms of a disorder or condition that can be treated by inhibiting LRRK-2 activity in a subject in need of treatment thereof by administering to the subject an effective amount of a compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d).

In a specific embodiment, for any method of treatment described above, the compound administered or used is a compound of formula (I) or a pharmaceutically acceptable salt thereof. In a specific embodiment, for any method of treatment described above, the compound administered or used is a compound of formula (II) or a pharmaceutically acceptable salt thereof. In a specific embodiment, for any method of treatment described above, the compound administered or used is a compound of formula (I-Z-1) or a pharmaceutically acceptable salt thereof. In a specific embodiment, for any method of treatment described above, the compound administered or used is a compound of formula (I-Z-2), formula (I-Z-2), formula (I-Z-2-a), formula (I-Z-2-b), formula (I-Z-2-c), or formula (I-Z-2-d) or a pharmaceutically acceptable salt thereof. In a specific embodiment, for any method of treatment described above, the compound administered or used is a compound of formula (I-Z-3-a) or a pharmaceutically acceptable salt thereof. In a specific embodiment, for any method of treatment described above, the compound administered or used is a compound of formula (I-Z-3-b) or a pharmaceutically acceptable salt thereof. In a specific embodiment, for any method of treatment described above, the compound administered or used is a compound of formula (I-Z-3-c) or a pharmaceutically acceptable salt thereof. In a specific embodiment, for any method of treatment described above, the compound administered or used is a compound of formula (I-Z-3-d) or a pharmaceutically acceptable salt thereof. In a specific embodiment, for any method of treatment described above, the compound administered or used is a compound of formula (I-I-1) or a pharmaceutically acceptable salt thereof. In a specific embodiment, for any method of treatment described above, the compound administered or used is a compound of formula (I-I-2) or a pharmaceutically acceptable salt thereof. In a specific embodiment, for any method of treatment described above, the compound administered or used is a compound of formula (I-D-1) or a pharmaceutically acceptable salt thereof. In a specific embodiment, for any method of treatment described above, the compound administered or used is a compound of formula (I-D-2) or a pharmaceutically acceptable salt thereof.

As used herein, a compound of formula (I) includes compounds of formulas (I), (I-A), (I-B), (I-C), (I-D), (I-E), (I-F), (I-G), (I-H), (I-I), (I-K), (I-L), (I-M), and (I-N). As used herein, a compound of Formula (I-D-2) includes compounds of formulas (I-H-2), (I-K-2) and (I-L-2). As used herein, a compound of formula (I-Z-1) includes compounds of formulas (I-Z-2), (I-D-1), (I-D-2), (I-Z-2-a), (I-Z-2-b), (I-Z-2-c), (I-Z-2-d), (I-Z-3-a), (I-Z-3-b), (I-Z-3-c), and (I-Z-3-d). As used herein a compound of formula (I-Z-2) includes compounds of formulas (I-D-2), (I-Z-2-a), (I-Z-2-b), (I-Z-2-c), (I-Z-2-d), (I-Z-3-a), (I-Z-3-b), (I-Z-3-c), and (I-Z-3-d).

In some embodiments of the methods described above, the compound which is administered to an individual is radio labeled.

In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. In some of such embodiments, the use of isotopically labeled compounds of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) allows for monitoring disease progression by use of brain imaging (e.g., by use of PET scans, CAT scans and the like).

In some embodiments, the isopically labeled compounds described herein serve as targeted LRRK engagement ligands. In other words, the use of such isotopically labeled compounds of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) provides a marker that allows for identification of patients having increased LRRK activity (e.g., increased LRRK2 activity due to mutated LRRK2). Accordingly, provided herein is a method of identifying an individual suffering from, or suspected to be suffering from a neurodegenerative disorder comprising administration of an isotopically labeled LRRK inhibitor (e.g., a compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d)) to the individual in need thereof and subjecting the individual to a brain scan (e.g, a PET scan or CAT scan or the like) wherein the detection of radioactivity in the brain identifies the individual as an individual having increased LRRK activity in the brain, and thereby identifies the individual as being susceptible to, or suffering from a neurodegenerative disorder.

Also provided herein is a method of identifying an individual suffering from, or suspected to be suffering from a neurodegenerative disorder comprising
(a) administering an isotopically labeled LRRK inhibitor to the individual; and
(b) imaging the brain of the individual to whom an isotopically labeled LRRK inhibitor has been administered with a brain scanning machine;
wherein the detection of radioactivity in the brain identifies the individual as being susceptible to, or suffering from a neurodegenerative disorder.

In some embodiments, the LRRK inhibitor is a compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d).

### Examples of Dosing and Treatment Regimens

A LRRK2 modulator (e.g., a LRRK inhibitor or partial inhibitor of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) is optionally used in the preparation of medicaments for treating any of the diseases or conditions described herein in an individual in need of such treatment, and involves administration of pharmaceutical compositions containing at least one compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

In the case wherein the patient's condition does not improve, upon the doctor's discretion the administration of the LRRK modulator (e.g., a LRRK inhibitor or partial inhibitor of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d)) is optionally continued chronically and/or at a higher dose, including throughout the duration of the patient's life in order to ameliorate or otherwise control or limit the neurodegeneration.

In the case wherein the patient's status does improve, upon the doctor's discretion the administration of the LLRK modulator (e.g., a LRRK inhibitor or partial inhibitor of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d)) is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the neurodegeneration, to a level at which the improved disease is retained. In some embodiments, patients require intermittent treatment on a long-term basis upon any recurrence of symptoms and/or progressive degeneration and/or recurrence.

In some embodiments, the pharmaceutical compositions described herein are in unit dosage forms suitable for single administration of precise dosages. In unit dosage form, the formulation is divided into unit doses containing appropriate quantities of one or more compound of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d). In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for the compounds are from about 0.1 mg to about 3000 mg, conveniently administered in divided doses, including, but not limited to, up to four times a day or in extended release form. Suitable unit dosage forms for oral administration include from about 0.1 to 1000 mg active ingredient, from about 0.1 to 500 mg active ingredient, from about 1 to 250 mg of active ingredient, or from about 1 to about 100 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the inhibitor used, the extent of neurodegenration, the mode of administration, the requirements of an individual, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. Compounds exhibiting high therapeutic indices are preferred. The data obtained from cell culture assays and animal studies is optionally used in formulating a range of dosage for use in human. The dosage of such compounds of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) lies preferably within a range of circulating concentrations that include the ED50 with minimal toxicity. The dosage optionally varies within this range depending upon the dosage form employed and the route of administration utilized.

### Assays

Animal Model

A modification of a transgenic mouse model with a mutant LRRK2 construct in a BAC expression vector described by Li et al. (Neurosci. 2009 July; 12(7):826-8) is used to test neuroprotective effect of compounds described herein.

The transgenic model expresses about 5-fold endogenous LRRK2 levels under the control of the prion promoter, so that it has high levels of expression in the hindbrain. There is a progressive behavioral phenotype beginning about 9 months and progressing to 15 months. Initially, beginning at 6-9 months, there is an increase in activity in the open field apparatus, but then after 9 months there is a progressive decrease in activity. In addition, a rotor-rod deficit appears at about 12 months. These two behavioral phenotypes are progressive, and provide readouts for therapeutic trials. In addition, tyrosine hydroxylase immunoreactivity is quantified. There is a significant decrease in TH-positive neurites in the substania nigra pars reticulata. Early on (at 9 months) there is no significant change in the number of TH-positive neurons. TH neuronal counts are also quantified at later time points.

As the model shows progressive behavioral changes beginning at about 9 or 10 months and progressing at least to 15 months, a drug is administered beginning at 10 months and the animals are tested at months 12 and 14. If there is a trend for significant difference, the experiment is continued to month 16. 12-15 mice per group are used to detect a 50% slowing of progression based on behavioral data.

PET Scans

Compounds of formula (I), formula (II), formula (I-D-1), formula (I-D-2), formula (I-I-1), formula (I-I-2), formula (I-Z-1), formula (I-Z-2), formula (I-Z-2-a), formula (I-Z-2-b), formula (I-Z-2-c), formula (I-Z-2-d), formula (I-Z-3-a), formula (I-Z-3-b), formula (I-Z-3-c), or formula (I-Z-3-d) are used to produce LRRK2 target engagement ligands (e.g., an isotopically labeled ligand) for use in PET imaging studies pre-clinically and/or clinically.

In Vitro Assays

High throughput in vitro assays are used to determine activity of compounds described herein. An exemplary assay is described in Example 3 below.

### EXAMPLES

### Example 1

### Synthesis of Intermediates

### Intermediate 1: 4-Methyl-1-trityl-1H-imidazole-2-carbaldehyde

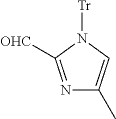

### Step a: 4-methyl-1-trityl-1H-imidazole

Trityl chloride (8.55 g, 30.7 mmol), Et₃N (7.12 mL, 51.2 mmol) and DMAP (0.16 g, 0.1 mmol) were added to a solution of 4-methyl-1H-imidazole (2.1 g, 25.6 mmol) in DCM and DMF (65.0 mL, 12:1). The reaction mixture was stirred at room temperature overnight. The mixture was treated with saturated NH₄Cl solution and extracted with ethyl acetate. The combined organic layers were washed with brine, dried over anhydrous Na₂SO₄ and concentrated to afford 6.3 g of the title compound (76% yield).

### Step b: 4-methyl-1-trityl-1H-imidazole-2-carbaldehyde

n-BuLi (2.5 M solution, 4.8 mL, 12.0 mmol) was added to a solution of 4-methyl-1-trityl-1H-imidazole (3.24 g, 10 mmol) in THF (18.0 mL) at −78° C. After the mixture was stirred at −78° C. for 1 h, DMF (2.36 mL, 31.5 mmol) was added. The reaction mixture was stirred at −78° C. for 30 min and then allowed to warm to room temperature. The pH value was adjusted to 1 with concentrated HCl solution and the resulting mixture was stirred for 2 h. Then the pH value was adjusted to 8 with aqueous NaHCO₃ solution. The mixture was extracted with EtOAc and the combined organic phases were washed with brine, dried over anhydrous Na₂SO₄ and concentrated to afford 2.0 g of the title compound (57% yield).

### Intermediate 2: 2-Methyl-1H-imidazole-5-carbaldehyde

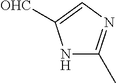

### Step a: N,N,2-trimethyl-1H-imidazole-1-sulfonamide

N,N-dimethylsulfamoyl chloride (7.15 g, 50 mmol) and Et₃N (3.99 mL, 28.7 mmol) were added to a solution of 2-methylimidazole (2.05 g, 25 mmol) in 1,2-dichloroethane (28.0 mL) and the reaction mixture was stirred at room temperature overnight. The solid was removed by filtration and washed with 1,2-dichloroethane. The combined filtrates were washed with saturated Na₂CO₃ solution, dried over anhydrous Na₂SO₄ and concentrated. Distillation of the residue afforded 2.8 g of the title compound (59% yield). ¹H NMR (300 MHz, CDCl₃): δ 7.11 (d, J=1.8 Hz, 1H), 6.78 (d, J=1.8 Hz, 1H), 2.82 (s, 6H), 2.61 (s, 3H). MS (EI): m/z 190 [M+H]⁺.

### Step b: 2-Methyl-1H-imidazole-5-carbaldehyde

n-BuLi (2.5 M solution, 2.4 mL, 6.0 mmol) was added to a solution of N,N,2-trimethyl-1H-imidazole-1-sulfonamide (0.95 g, 5.0 mmol) in THF (40.0 mL) at −78° C. After the mixture was stirred at −78° C. for 30 min, DMF (2.36 mL, 31.5 mmol) was added. The reaction mixture was stirred at −78° C. for 1 h and then allowed to warm to room temperature. The pH value was adjusted to 1 with concentrated HCl solution and the resulting mixture was stirred for 2 h. Then the pH value was adjusted to 8 with aqueous NaHCO₃ solution. The mixture was extracted with EtOAc and the combined organic phases were washed with brine, dried over anhydrous Na₂SO₄ and concentrated to afford 0.33 g of the title compound (60% yield). ¹H NMR (300 MHz, DMSO-d₆): δ 12.6 (br s, 1H), 9.60 (s, 1H), 7.85 (s, 1H), 2.32 (s, 3H).

The following intermediate was similarly prepared from the corresponding imidazole:

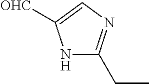

2-Ethyl-1H-imidazole-5-carbaldehyde.

### Intermediate 3: 1H-Pyrrolo[2,3-b]pyridine-2-carbaldehyde

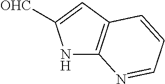 [SMILES: O=Cc1cc2cccnc2[nH]1]

### Step a: 2-bromonicotinaldehyde

n-BuLi (2.5 M solution, 42.0 mL, 0.105 mol) was added to a solution of diisopropylamine (15.5 mL, 0.11 mol) in THF (180.0 mL) at −78° C. After the mixture was stirred for 10 min, a solution of 2-bromopyridine (15.8 g, 0.10 mol) in THF (20.0 mL) was added dropwise and the mixture was stirred at −78° C. for 2 h. Then HCO₂Et (14.8 g, 0.20 mol) was added dropwise and the reaction mixture was stirred at −78° C. for another hour. The mixture was allowed to warm to −30° C. and saturated NH₄Cl solution was added. The resulting mixture was extracted with EtOAc and the combined organic phases were washed with brine, dried over anhydrous Na₂SO₄ and concentrated. The residue was purified by flash column chromatography (PE/EA=15:1) to afford 2.0 g of the title compound as a light yellow solid (11% yield).

### Step b: ethyl 1H-pyrrolo[2,3-b]pyridine-2-carboxylate

A mixture of 2-bromonicotinaldehyde (1.0 g, 5.4 mmol), ethyl 2-cyanoacetate (0.69 g, 6.0 mmol), CuI (115 mg, 0.6 mmol) and Cs₂CO₃ (3.59 g, 11.0 mmol) in DMF (10.0 mL) was stirred at 50° C. for 3 h. After the mixture was cooled to room temperature, it was poured into saturated NH₄Cl solution (50.0 mL). The resulting mixture was extracted with DCM and the combined organic layers were washed with brine, dried over anhydrous Na₂SO₄ and concentrated. The residue was purified by flash column chromatography (PE/EA/DCM=80/10/5 to 60/10/10) to afford 0.35 g of the title compound as a yellow solid (35% yield).

### Step c: 1H-pyrrolo[2,3-b]pyridine-2-carbaldehyde

A solution of ethyl 1H-pyrrolo[2,3-b]pyridine-2-carboxylate (190 mg, 1.0 mmol) in THF (5.0 mL) was added dropwise to a suspension of LiAlH₄ (42 mg, 1.1 mmol) in THF (10.0 mL) at −20° C. and the reaction mixture was stirred at −20° C. for 2 h. The reaction was quenched with saturated NH₄Cl solution (10.0 mL). The resulting mixture was filtered and the filtration cake was washed with ethyl acetate. The combined filtrates were washed with brine, dried over anhydrous Na₂SO₄ and concentrated to afford 120 mg of the title compound (67% yield), which was used as such in the next step.

### Intermediate 4: 3-Methyl-1H-pyrazole-5-carbaldehyde

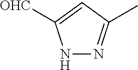

### Step a: 5-(Diethoxymethyl)-3-methyl-1H-pyrazole

1,1-Diethoxypentane-2,4-dione (3.2 g, 17.0 mmol) was added dropwise to a solution of hydrazine sulfate (2.21 g, 17.0 mmol) in aqueous NaOH solution (10%, 15.0 mL) at 10-15° C. and the reaction mixture was stirred at 10-15° C. for 1 h. The mixture was extracted with Et₂O and the combined extracts were washed with brine, dried over anhydrous Na₂SO₄ and concentrated to give 2.2 g of the title compound as a colorless oil (70% yield).

### Step b: 3-Methyl-1H-pyrazole-5-carbaldehyde

A solution of 5-(diethoxymethyl)-3-methyl-1H-pyrazole (2.2 g, 12.0 mmol)) in aqueous HCl solution (1%, 20.0 mL) was stirred at room temperature overnight. The precipitate was collected by filtration, washed with water and cold methanol and dried to give 1.0 g of the title compound as a white solid (76% yield).

### Intermediate 5: 1,3-Benzothiazole-2-carbaldehyde

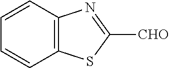 [SMILES: O=Cc1nc2ccccc2s1]

n-BuLi (2.5 M, 5.2 mL, 13 mmol) was added to a solution of benzo[d]thiazole (1.35 g, 10.0 mmoL) in THF (45.0 mL) at −78° C. and the mixture was stirred at −78° C. for 30 min. DMF (3.10 mL, 40.0 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated to afford 612 mg of the title compound as a yellow solid (37% yield).

### Intermediate 6: Quinoline-3-carbaldehyde

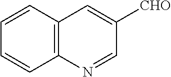 [SMILES: O=Cc1cnc2ccccc2c1]

### Step a: N-methoxy-N-methylquinoline-3-carboxamide

To a solution of quinoline-3-carboxylic acid (519 mg, 3.0 mmol) and N,O-dimethylhydroxylamine hydrochloride (366 mg, 3.75 mmol) in DCM/DMF (1:1, 10.0 mL) were added EDCI (719 g, 3.75 mmol) and HOBt (527 mg, 3.9 mmol) followed by addition of DIPEA (2.61 mL, 15.0 mmol). The reaction mixture was stirred at room temperature for 12 h. The mixture was diluted with water and extracted with DCM. The combined organic extracts were washed with brine, dried over anhydrous Na₂SO₄ and concentrated. The residue was purified by flash column chromatography on silica gel (PE/EA=5:1 to 1:1) to afford 324 mg of the title compound as a colorless liquid (50% yield).

### Step b: quinoline-3-carbaldehyde

A solution of N-methoxy-N-methylquinoline-3-carboxamide (324 mg, 1.5 mmol) in THF (10.0 mL) was added to a suspension of LiAlH₄ (63 mg, 1.65 mol) in THF (2.0 mL) and the reaction mixture was stirred at 0° C. for 2 h. The reaction was quenched with saturated aqueous NH₄Cl solution (5.0 mL) and then stirred at room temperature for 30 min. The mixture was filtered and the filtration cake was washed with ethyl acetate. The combined filtrates were washed with brine, dried over anhydrous Na₂SO₄ and concentrated to afford 180 mg of the title compound as a red oil (80% yield), which was used for the next step without purification.

### Intermediate 7: 1,3-Benzoxazole-2-carbaldehyde

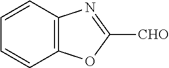 [SMILES: O=Cc1nc2ccccc2o1]

### Step a: N-(2-hydroxyphenyl)acetamide

Ac₂O (6.13 g, 60.0 mmol) was added dropwise to a mixture of 2-aminophenol (5.45 g, 50.0 mmol) in HOAc (35.0 mL) and the reaction mixture was refluxed for 1.5 h. The mixture was cooled to room temperature and pounded into water (150 mL). The precipitate was collected by filtration, washed with water and PE, dried under vacuum to afford 6.5 g of the title compound as a white solid (86% yield).

### Step b: 2-methylbenzo[d]oxazole

To a solution of N-(2-hydroxyphenyl)acetamide (3.02 g, 20.0 mmol) in CHCl₃ (15.0 mL) was added POCl₃ (4.60 g, 30.0 mmol) dropwise and the reaction mixture was refluxed for 1 h. The mixture was cooled to room temperature and poured into ice-water (100 mL). After the resulting mixture was adjusted to pH=8 with aqueous Na₂CO₃ solution, it was extracted with DCM. The combined organic layers were washed with brine, dried over anhydrous Na₂SO₄ and concentrated to afford 1.33 g of the title compound (50% yield), which was used for the next step without purification.

### Step c: benzo[c/]oxazole-2-carbaldehyde

A mixture of 2-methylbenzo[d]oxazole (1.33 g, 10.0 mmol) and DMF-DMA (4.65 mL, 35.0 mmol) in DMF (40.0 mL) containing piperidine (0.1 mL) was heated under reflux for 2 h. After the mixture containing compound 10 was cooled to room temperature, a mixture of NaIO₄ (6.42 g, 30.0 mmol) in H₂O/DMF (5:1, 60.0 mL) was added and the reaction mixture was stirred at room temperature for 1 h. The mixture was extracted with ethyl acetate and the combined extracts were washed with brine, dried over anhydrous Na₂SO₄ and concentrated. The residue was purified by flash column chromatography on silica gel (PE/EA=12:1 to 6:1) to afford 200 mg of the title compound as a yellow solid (14% yield).

### Intermediate 8: 2-Isopropyl-1H-imidazole-5-carbaldehyde

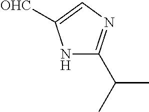

### Step a: 2-isopropyl-N,N-dimethyl-1H-imidazole-1-sulfonamide

2-Isopropylimidazole (2.0 g, 20.0 mmol) was added to a suspension of 60% sodium hydride (2.4 g, 60.0 mmol) in DMF (20.0 mL) at 0° C. and the mixture was stirred for 0.5 h. Then N,N-dimethylsulfamoyl chloride (3.4 g, 24.0 mmol) was added dropwise and the reaction mixture was stirred at room temperature for 2 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (DCM/MeOH=50:1 to 10:1) to afford 1.48 g of the title compound as a white solid (68% yield).

### Step b: 2-isopropyl-1H-imidazole-5-carbaldehyde

n-BuLi (2.5 M, 3.3 mL, 8.2 mmol) was added to a solution of 2-isopropyl-N,N-dimethyl-1H-imidazole-1-sulfonamide (1.48 g, 6.8 mmol) in THF (40.0 mL) at −78° C. and the mixture was stirred at −78° C. for 30 min. DMF (3.33 mL, 41.3 mmol) was added and the reaction mixture was stirred at −78° C. for 1 h and allowed to warm to room temperature over 1 h. The mixture was adjusted to pH=1 with concentrated HCl and stirred for 2 h. Then the mixture was adjusted to pH=8 with aqueous NaHCO₃ solution and extracted with ethyl acetate. The combined extracts were washed with brine, dried over anhydrous Na₂SO₄ and concentrated. The residue was purified by flash column chromatography on silica gel (PE/EA=10:1 to 5:1) to afford 450 mg of the title compound as a white solid (48% yield).

### Intermediate 9: 2-tert-Butyl-1H-imidazole-5-carbaldehyde

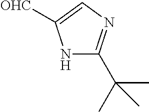

### Step a: Ethyl Pivalimidate Hydrochloride

A mixture of pivalonitrile (2.0 g, 24 mmol) in HCl/EtOH (5.0 N, 20.0 mL) was stirred at room temperature overnight. The mixture was concentrated to give 3.5 g of the title compound as a white solid (87% yield).

### Step b: 2-tert-butyl-1H-imidazole

A solution of ethyl pivalimidate hydrochloride (4.2 g, 25.2 mmol) and aminoacetaldehyde dimethyl acetal (3.2 g, 30.45 mmol) in methanol (8.0 mL) was stirred at room temperature overnight. The mixture was concentrated and concentrated HCl (5.0 mL) was added. The mixture was concentrated again and water (10.0 mL) was added. The mixture was adjusted to pH=11 with K₂CO₃ solution and ethanol (10.0 mL) was added. The resulting mixture was filtered and the filtrate was concentrated. The residue was purified by flash column chromatography on silica gel (DCM/MeOH=100:1 to 50:1) to afford 2.3 g of the title compound as a white solid (73% yield).

### Step c: 2-tert-butyl-N,N-dimethyl-1H-imidazole-1-sulfonamide

2-tert-Butyl-1H-imidazole (0.5 g, 4.03 mmol) was added to a suspension of 60% sodium hydride (0.5 g, 12.1 mmol) in DMF (6.0 mL) at 0° C. and the mixture was stirred for 0.5 h. Then N,N-dimethylsulfamoyl chloride (0.7 g, 4.84 mmol) was added dropwise and the reaction mixture was stirred at room temperature for 2 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (DCM/MeOH=20:1 to 5:1) to afford 0.8 g of the title compound as a white solid (86% yield).

### Step d: 2-tert-butyl-5-formyl-N,N-dimethyl-1H-imidazole-1-sulfonamide

n-BuLi (2.5 M, 1.1 mL, 2.6 mmol) was added to a solution of 2-tert-butyl-N,N-dimethyl-1H-imidazole-1-sulfonamide (0.4 g, 1.73 mmol) in THF (5.0 mL) at −78° C. and the mixture was stirred at −78° C. for 30 min. DMF (0.85 mL, 10.9 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated to afford 0.7 g of the title compound as a yellow oil (78% yield).

### Step e: 2-tert-butyl-1H-imidazole-5-carbaldehyde

Concentrated HCl (1.0 mL) was added to a solution of 2-tert-butyl-5-formyl-N,N-dimethyl-1H-imidazole-1-sulfonamide (0.7 g, 2.7 mmol) in THF (5.0 mL) and the reaction mixture was stirred at room temperature for 1 h. The mixture was adjusted to pH=8 with saturated NaHCO₃ solution and extracted with ethyl acetate. The combined extracts were washed with brine, dried over anhydrous Na₂SO₄ and concentrated to afford 0.7 g of the title compound as a yellow solid (59% yield).

The following intermediate was prepared in a similar way starting from the corresponding alkyl nitrile:

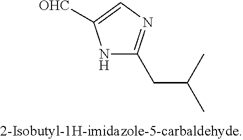

### Intermediate 10: 2-(Methoxymethyl)-1H-imidazole-5-carbaldehyde

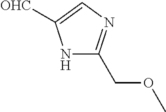

### Step a: 2-(hydroxymethyl)-N,N-dimethyl-1H-imidazole-1-sulfonamide

NaBH₄ (0.28 g, 7.5 mmol) was added to a solution of 2-formyl-N,N-dimethyl-1H-imidazole-1-sulfonamide (0.5 g, 2.5 mmol) in methanol (20.0 mL) and the reaction mixture was stirred at room temperature for 3 h. Saturated NH₄Cl solution was added and the resulting mixture was extracted with ethyl acetate. The combined extracts were washed with brine, dried over anhydrous Na₂SO₄ and concentrated to afford 0.4 g of the title compound as a white solid (78% yield).

### Step b: 2-(methoxymethyl)-N,N-dimethyl-1H-imidazole-1-sulfonamide

A mixture of 2-(hydroxymethyl)-N,N-dimethyl-1H-imidazole-1-sulfonamide (0.4 g, 1.95 mmol) and 60% sodium hydride (134 mg, 5.85 mmol) in THF (15.0 mL) was stirred at 0° C. for 15 min. Then CH₃I (0.47 g, 3.3 mmol) was added dropwise and the reaction mixture was allowed to warm to room temperature and stirred for 2 h. Saturated NH₄Cl solution was added and the resulting mixture was extracted with ethyl acetate. The combined organic layers were washed with brine, dried over anhydrous Na₂SO₄ and concentrated. The residue was purified by flash column chromatography on silica gel (DCM/MeOH=20:1 to 5:1) to afford 0.8 g of the title compound as a yellow solid (87% yield).

### Step c: 2-(methoxymethyl)-1H-5-formyl-N,N-dimethyl-1H-imidazole-1-sulfonamide

n-BuLi (2.5 M, 1.8 mL, 2.6 mmol) was added to a solution of 2-(methoxymethyl)-N,N-dimethyl-1H-imidazole-1-sulfonamide (0.8 g, 3.8 mmol) in THF (20.0 mL) at −78° C. and the mixture was stirred at −78° C. for 30 min. DMF (6.08 mL, 24.0 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated to afford 0.78 g of the title compound as a yellow oil (87% yield).

### Step d: 2-(methoxymethyl)-1H-imidazole-5-carb aldehyde

Concentrated HCl (1.0 mL) was added to a solution of 2-(methoxymethyl)-1H-5-formyl-N,N-dimethyl-1H-imidazole-1-sulfonamide (680 mg, 2.75 mmol) in THF (5.0 mL) and the reaction mixture was stirred at room temperature for 1 h. The mixture was adjusted to pH=8 with saturated NaHCO₃ solution and extracted with ethyl acetate. The combined extracts were washed with brine, dried over anhydrous Na₂SO₄ and concentrated. The residue was purified by flash column chromatography on silica gel (PE/EA=5:1) to afford 68 mg of the title compound as a white solid (8% yield).

### Intermediate 11: 2-(Tetrahydro-2H-pyran-4-yl)-1H-imidazole-5-carbaldehyde

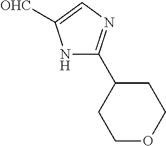 [SMILES: O=Cc1cnc(C2CCOCC2)[nH]1]

### Step a: tetrahydro-2H-pyran-4-carbonitrile

A solution of dihydro-2H-pyran-4(3H)-one (10.0 g, 0.1 mol) and TosMIC (25.35 g, 0.13 mol) in DME (75.0 mL) was cooled to −10° C. and t-BuOK (28.0 g, 0.25 mol) was added in portions to keep the temperature below 5° C. The reaction mixture was stirred at 0° C. for 1 h and then at room temperature for 2 h. The solvent was removed under reduced pressure and the residue was treated with water. The resulting mixture was extracted with ether and the combined organic layers were washed with brine, dried over anhydrous Na₂SO₄ and concentrated. The residue was purified by distillation to afford 18 g of the title compound as a light yellow liquid (73% yield).

### Step b: ethyl tetrahydro-2H-pyran-4-carbimidate hydrochloride

A solution of tetrahydro-2H-pyran-4-carbonitrile (3.0 g, 27.0 mmol) in ethanol (20.0 mL) was cooled to −20° C. HCl gas was bubbled into the solution for 1 h and the reaction mixture was stirred at room temperature overnight. The mixture was concentrated to afford 7.5 g of the title compound as a brown solid (quant. yield).

### Step c: 2-(tetrahydro-2H-pyran-4-yl)-1H-imidazole

A solution of ethyl tetrahydro-2H-pyran-4-carbimidate hydrochloride (3.0 g, 15.6 mmol) and aminoacetaldehyde dimethyl acetal (1.83 g, 17.47 mmol) in methanol (4.0 mL) was stirred at room temperature overnight. The mixture was concentrated and concentrated HCl (5.0 mL) was added. The mixture was concentrated again and water (10.0 mL) was added. The mixture was adjusted to pH=11 with K₂CO₃ solution and ethanol (10.0 mL) was added. The resulting mixture was filtered and the filtrate was concentrated. The residue was purified by flash column chromatography on silica gel (DCM/MeOH=100:1 to 15:1) to afford 1.3 g of the title compound as a white solid (55% yield).

### Step d: 2-(tetrahydro-2H-pyran-4-yl)-N,N-dimethyl-1H-imidazole-1-sulfonamide

2-(Tetrahydro-2H-pyran-4-yl)-1H-imidazole (0.5 g, 3.3 mmol) was added to a suspension of 60% sodium hydride (234 mg, 9.9 mmol) in DMF (4.0 mL) at 0° C. and the mixture was stirred for 0.5 h. Then N,N-dimethylsulfamoyl chloride (0.49 g, 3.5 mmol) was added dropwise and the reaction mixture was stirred at room temperature for 2 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (DCM/MeOH=20:1 to 5:1) to afford 0.6 g of the title compound as a white solid (70% yield).

### Step e: 2-(tetrahydro-2H-pyran-4-yl)-5-formyl-N,N-dimethyl-1H-imidazole-1-sulfonamide

n-BuLi (2.5 M, 0.92 mL, 2.3 mmol) was added to a solution of 2-(tetrahydro-2H-pyran-4-yl)-N,N-dimethyl-1H-imidazole-1-sulfonamide (0.5 g, 1.9 mmol) in THF (10.0 mL) at −78° C. and the mixture was stirred at −78° C. for 30 min. DMF (3.04 mL, 12.0 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated to afford 0.5 g of the title compound as a yellow solid (92% yield).

### Step f: 2-(tetrahydro-2H-pyran-4-yl)-1H-imidazole-5-carbaldehyde

Concentrated HCl (1.0 mL) was added to a solution of 2-(tetrahydro-2H-pyran-4-yl)-5-formyl-N,N-dimethyl-1H-imidazole-1-sulfonamide (0.5 g, 1.7 mmol) in THF (5.0 mL) and the reaction mixture was stirred at room temperature for 1 h. The mixture was adjusted to pH=8 with saturated NaHCO₃ solution and extracted with ethyl acetate. The combined extracts were washed with brine, dried over anhydrous Na₂SO₄ and concentrated to afford 0.7 g of the title compound as a white solid (33% yield).

### Intermediate 12: 2-(2,6-Difluorophenyl)-1H-imidazole-5-carbaldehyde

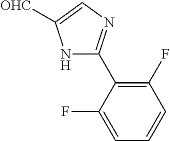 [SMILES: O=Cc1cnc(-c2c(F)cccc2F)[nH]1]

### Step a: 2,6-difluorobenzimidamide

n-BuLi (2.5 M, 14.4 mL, 36.0 mmol) was added to a solution of HMDS (7.2 mL, 34.5 mmol) in THF (50.0 mL) at 0° C. and the mixture was stirred at 0° C. for 30 min. 2,6-Difluorobenzonitrile (2.0 g, 14.4 mmol) was added and the reaction mixture was stirred at 0° C. for 30 min and then allowed to warm to room temperature over 4 h. The mixture was poured into concentrated HCl (60.0 mL) and extracted with ethyl acetate. The aqueous layer was adjusted to pH=10 with NaOH solution (6.0 N) and extracted with DCM. The combined extracts were washed with brine, dried over anhydrous Na₂SO₄ and concentrated to afford 1.1 g of the title compound as a yellow solid (49% yield).

### Step b: (2-(2,6-difluorophenyl)-1H-imidazol-5-yl)methanol

A mixture of 2,6-difluorobenzimidamide (1.1 g, 7.05 mmol), 1,3-dihydroxypropan-2-one (1.27 g, 14.1 mmol) and NH₄Cl (1.51 g, 28.4 mmol) in NH₃—H₂O (15.0 mL) was refluxed for 1 h. After the mixture was cooled to room temperature, it was pound into water and the resulting mixture was extracted with DCM. The combined extracts were washed with brine, dried over anhydrous Na₂SO₄ and concentrated. The residue was recrystallized from hexane/ethyl acetate to afford 0.15 g of the title compound as a yellow solid (10% yield).

### Step c: 2-(2,6-difluorophenyl)-1H-imidazole-5-carbaldehyde

To a solution of (2-(2,6-difluorophenyl)-1H-imidazol-5-yl)methanol (0.1 g, 0.47 mmol) in DCM (5.0 mL) was added PCC (0.26 g, 1.19 mmol) and the reaction mixture was stirred at room temperature for 30 min. Water was added and the resulting mixture was extracted with DCM. The combined extracts were washed with brine, dried over anhydrous Na₂SO₄ and concentrated to afford 50 mg of the title compound as a colorless oil (50% yield).

### Intermediate 13: 2-(Morpholinomethyl)-1H-imidazole-5-carbaldehyde

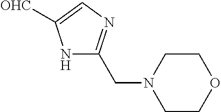 [SMILES: O=Cc1cnc(CN2CCOCC2)[nH]1]

### Step a: 4-((1H-imidazol-2-yl)methyl)morpholine

A mixture of 1H-imidazole-2-carbaldehyde (0.5 g, 5.2 mmol), morpholine (0.9 g, 10.4 mmol) and NaBH₄ (0.6 mg, 0.2 mmol) in MeOH (20.0 mL) was heated at 60° C. for 2 h. After the mixture was cooled to room temperature, it was concentrated under reduced pressure. The residue was partitioned between DCM and water and the separated organic layer was washed with brine, dried over anhydrous Na₂SO₄ and concentrated. The residue was purified by flash column chromatography on silica gel (DCM/MeOH=100:1 to 50:1) to afford 0.5 g of the title compound as a white solid (57% yield).

### Step b: N,N-dimethyl-2-(morpholinomethyl)-1H-imidazole-1-sulfonamide

4-((1H-Imidazol-2-yl)methyl)morpholine (0.45 g, 2.69 mmol) was added to a suspension of 60% sodium hydride (0.16 g, 4.04 mmol) in DMF (10.0 mL) at 0° C. and the mixture was stirred for 0.5 h. Then N,N-dimethylsulfamoyl chloride (0.46 g, 3.23 mmol) was added dropwise and the reaction mixture was stirred at room temperature for 2 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (PE/EA=5:1 to 1:1) to afford 0.54 g of the title compound as a colorless oil (73% yield).

### Step c: 2-(morpholinomethyl)-1H-imidazole-5-carbaldehyde

n-BuLi (2.5 M, 1.0 mL, 2.4 mmol) was added to a solution of N,N-dimethyl-2-(morpholinomethyl)-1H-imidazole-1-sulfonamide (0.54 g, 2.0 mmol) in THF (20.0 mL) at −78° C. and the mixture was stirred at −78° C. for 30 min. DMF (0.93 mL, 12.0 mmol) was added and the reaction mixture was stirred at −78° C. for 1 h and then allowed to warm to room temperature over 1 h. The mixture was adjusted to pH=1 with concentrated HCl and stirred for 2 h. Then the mixture was adjusted to pH=8 with aqueous NaHCO₃ solution and extracted with ethyl acetate. The combined extracts were washed with brine, dried over anhydrous Na₂SO₄ and concentrated. The residue was purified by flash column chromatography on silica gel (PE/EA=10:1 to 5:1) to afford 272 mg of the title compound as a yellow solid (71% yield).

### Intermediate 14: 2-(2-Methoxyethyl)-1H-imidazole-5-carbaldehyde

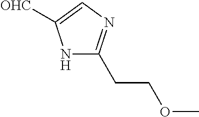

### Step a: ethyl 3-methoxypropanimidate hydrochloride

A mixture of 3-methoxypropanenitrile (2.4 g, 24.0 mmol) in 40% HCl-EtOH (10.0 mL) was stirred at room temperature overnight. The mixture was concentrated to give a quantitative amount of the title compound as a viscous oil, which was used directly without further purification.

### Step b: 2-(2-methoxyethyl)-1H-imidazole

A solution of ethyl 3-methoxypropanimidate hydrochloride and 2,2-diethoxyethanamine (5.2 mL, 48.0 mmol) in ethanol (10.0 mL) was stirred at room temperature overnight. After the mixture was concentrated, HCl solution (1.3 N, 20.0 mL) was added and the mixture was stirred at room temperature overnight. K₂CO₃ was added slowly to adjust pH=10 and the resulting mixture was extracted with DCM. The combined organic layers were washed with brine, dried over anhydrous Na₂SO₄ and concentrated. The residue was purified by flash column chromatography on silica gel (DCM/MeOH=20:1) to afford 1.5 g of the title compound as a yellow oil (24% yield in two steps).

### Step c: 2-(2-methoxyethyl)-N,N-dimethyl-1H-imidazole-1-sulfonamide

N,N-Dimethylsulfamoyl chloride (1.6 mL, 14.6 mmol) was added to a solution of 2-(2-methoxyethyl)-1H-imidazole (1.5 g, 12.1 mmol) in acetonitrile (210.0 mL) containing K₂CO₃ (2.5 g, 18.3 mmol) and the reaction mixture was stirred at 70° C. for 7 h. The mixture was cooled to room temperature and water (150 mL) was added. The resulting mixture was extracted with ethyl acetate and the combined organic layers were washed with brine, dried over anhydrous Na₂SO₄ and concentrated to afford a quantitative amount of the title compound, which was used directly without further purification.

### Step d: 2-(2-methoxyethyl)-1H-imidazole-5-carbaldehyde

n-BuLi (2.5 M, 5.4 mL, 13.4 mmol) was added to a solution of 2-(2-methoxyethyl)-N,N-dimethyl-1H-imidazole-1-sulfonamide (2.8 g, 12.1 mmol) in THF (60.0 mL) at −78° C. and the mixture was stirred at −78° C. for 30 min. DMF (5.6 mL, 72.8 mmol) was added and the reaction mixture was stirred at −78° C. for 1 h and allowed to warm to room temperature over 1 h. The mixture was adjusted to pH=1 with concentrated HCl and stirred for 2 h. Then the mixture was adjusted to pH=8 with aqueous NaHCO₃ solution and extracted with ethyl acetate. The combined extracts were washed with brine, dried over anhydrous Na₂SO₄ and concentrated to give the title compound, which was used directly without further purification.

### Intermediate 15: 3-Ethyl-5-formyl-N,N-dimethyl-4H-1,2,4-triazole-4-sulfonamide

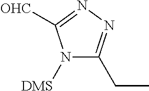

### Step a: Propionamide

A mixture of propionyl chloride (5.0 ml, 57.5 mmol) and NH₃.H₂O (80.0 mL) was stirred at room temperature for 0.5 h. The mixture was concentrated and the residue was treated with ethanol and filtered. The filtrate was concentrated to afford 3.44 g of the title compound (87% yield).

### Step b: 3-ethyl-4H-1,2,4-triazole

A solution of propionamide (3.44 g, 47.1 mmol) in DMF-DMA (120.0 mL) was heated at 120° C. for 2 h. The mixture was cooled to room temperature and then concentrated under reduced pressure to afford a yellow oil. The oil was dissolved in HOAc (100.0 mL) and hydrazine hydrate (10.0 mL) was added dropwise. The reaction mixture was heated at 90° C. for 2 h and then cooled to room temperature. The mixture was concentrated under reduced pressure and the residue was treated with saturated K₂CO₃ solution (pH=8). The resulting mixture was extracted with DCM and the combined extracts were washed with brine, dried over anhydrous Na₂SO₄ and concentrated. The residue was purified by flash column chromatography on silica gel (PE/EA=5:1 to 3:1) to provide 2.65 g of the title compound as a yellow oil (59% yield).

### Step c: 3-ethyl-N,N-dimethyl-4H-1,2,4-triazole-4-sulfonamide

Sodium hydride (60%, 0.526 g, 13.1 mmol) was added to a solution of 3-ethyl-4H-1,2,4-triazole (2.28 g, 8.76 mmol) in DMF (20.0 mL) at 0° C. and the mixture was stirred for 0.5 h. Then N,N-dimethylsulfamoyl chloride (1.51 g, 10.5 mmol) was added dropwise and the reaction mixture was stirred at room temperature for 2 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (PE/EA=10:1 to 2:1) to afford 1.05 g of the title compound as a yellow oil (59% yield).

### Step d: 3-ethyl-5-formyl-N,N-dimethyl-4H-1,2,4-triazole-4-sulfonamide

n-BuLi (2.5 M, 1.47 mL, 3.68 mmol) was added to a solution of 3-ethyl-N,N-dimethyl-4H-1,2,4-triazole-4-sulfonamide (0.50 g, 2.45 mmoL) in THF (15.0 mL) at −78° C. and the mixture was stirred at −78° C. for 30 min. DMF (0.76 mL, 10.0 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated. The residue was purified by flash column chromatography (PE/EA=10:1 to 2:1) to afford 170 mg of the title compound as a white solid (30% yield).

### Intermediate 16: 3-Isopropyl-4H-1,2,4-triazole-5-carbaldehyde

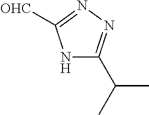

### Step a: Isobutyramide

A mixture of isobutyric acid (10.0 g, 114.0 mmol), EDCI (24.0 g, 125.9 mmol), HOBt (17.0 g, 125.9 mmol) and NH₃—H₂O (13.0 mL) in DMF (300.0 mL) was stirred at 0° C. for 1 h. The mixture was poured into water (500 mL) and extracted with ethyl acetate. The combined extracts were washed with brine, dried over anhydrous Na₂SO₄ and concentrated. The residue was purified by flash column chromatography on silica gel (PE/EA=10:1 to 2:1) to afford 6.9 g of the title compound (69% yield).

### Step b: 3-isopropyl-4H-1,2,4-triazole

A solution of isobutyramide (3.9 g, 44.8 mmol) in DMF-DMA (110.0 mL) was heated at 120° C. for 2 h. The mixture was cooled to room temperature and then concentrated under reduced pressure to afford a yellow oil. The oil was dissolved in HOAc (100.0 mL) and hydrazine hydrate (10.0 mL) was added dropwise. The reaction mixture was heated at 90° C. for 2 h and then cooled to room temperature. The mixture was concentrated under reduced pressure and the residue was treated with saturated K₂CO₃ solution (pH=8). The resulting mixture was extracted with DCM and the combined extracts were washed with brine, dried over anhydrous Na₂SO₄ and concentrated. The residue was purified by flash column chromatography on silica gel (PE/EA=5:1 to 3:1) to provide 4.3 g of the title compound as a yellow oil (56% yield).

### Step c: N,N-dimethyl-3-isopropyl-4H-1,2,4-triazole-1-sulfonamide

NaH (60%, 1.07 g, 26.7 mmol) was added to a solution of 3-isopropyl-4H-1,2,4-triazole (2.28 g, 20.5 mmol) in DMF (45.0 mL) at 0° C. and the mixture was stirred for 0.5 h. Then N,N-dimethylsulfamoyl chloride (3.54 g, 24.6 mmol) was added dropwise and the reaction mixture was stirred at room temperature for 2 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (PE/EA=5:1 to 1:1) to afford 3.73 g of the title compound as a colorless oil (83% yield).

### Step d: 3-isopropyl-4H-1,2,4-triazole-5-carbaldehyde

n-BuLi (2.5 M, 3.58 mL, 8.94 mmol) was added to a solution of N,N-dimethyl-3-isopropyl-4H-1,2,4-triazole-1-sulfonamide (1.50 g, 6.88 mmoL) in THF (45.0 mL) at −78° C. and the mixture was stirred at −78° C. for 30 min. DMF (2.12 mL, 27.5 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated to give the intermediate. Concentrated HCl (3.0 mL) was added to a solution of the intermediate in THF (15.0 mL) and the reaction mixture was stirred at room temperature for 1 h. The mixture was adjusted to pH=8 with saturated NaHCO₃ solution and extracted with ethyl acetate. The combined extracts were washed with brine, dried over anhydrous Na₂SO₄ and concentrated to afford 0.5 g of the title compound (52% yield over two steps).

### Intermediate 17: 2-Morpholino-1H-imidazole-5-carbaldehyde

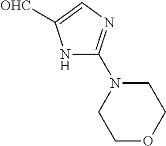 [SMILES: O=Cc1cnc(N2CCOCC2)[nH]1]

### Step a: 2-bromo-N,N-dimethyl-1H-imidazole-1-sulfonamide

n-BuLi (2.5 M, 16.5 mL, 2.75 mmol) was added to a solution of N,N-dimethyl-1H-imidazole-1-sulfonamide (6.0 g, 34.29 mmol) in THF (50.0 mL) at −78° C. and the mixture was stirred at −78° C. for 30 min. CBr₄ (12.5 g, 37.7 mmol) was added and the reaction mixture was stirred at −78° C. for 0.5 h and then allowed to warm to room temperature and stirred overnight. Water was added and the resulting mixture was extracted with ethyl acetate. The combined extracts were washed with brine, dried over anhydrous Na₂SO₄ and concentrated to afford 4.4 g of the title compound as a dark brown oil (51% yield).

### Step b: N,N-dimethyl-2-morpholino-1H-imidazole-1-sulfonamide

A mixture of 2-bromo-N,N-dimethyl-1H-imidazole-1-sulfonamide (3.9 g, 15.4 mmol) in morpholine (20.0 mL) was heated under reflux for 2 h. The mixture was concentrated and the residue was purified by flash column chromatography on silica gel (PE/EA=5:1 to 1:1) to afford 390 mg of the title compound as a colorless oil (10% yield).

### Step c: 2-morpholino-1H-imidazole-5-carbaldehyde

n-BuLi (2.5 M, 0.9 mL, 2.25 mmol) was added to a solution of N,N-dimethyl-2-morpholino-1H-imidazole-1-sulfonamide (390 mg, 1.5 mmol) in THF (5.0 mL) at −78° C. and the mixture was stirred at −78° C. for 30 min. DMF (0.7 mL, 9.45 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated to give the intermediate (141 mg). Concentrated HCl (1.0 mL) was added to a solution of the intermediate (141 mg, 0.49 mmol) in THF (5.0 mL) and the reaction mixture was stirred at room temperature for 1 h. The mixture was adjusted to pH=8 with saturated NaHCO₃ solution and extracted with ethyl acetate. The combined extracts were washed with brine, dried over anhydrous Na₂SO₄ and concentrated to afford 80 mg of the title compound as a yellow solid (29% yield over two steps).

### Intermediate 18: 2-(Dimethylamino)-1H-imidazole-5-carbaldehyde

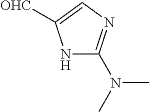

### Step a: 2-(dimethylamino)-N,N-dimethyl-1H-imidazole-1-sulfonamide

A solution of 2-bromo-N,N-dimethyl-1H-imidazole-1-sulfonamide (3.0 g, 11.8 mmol) in 33% dimethylamine (aqueous, 10.0 mL) was heated at 100° C. overnight in a high-pressure reactor. The mixture was cooled to room temperature and extracted with DCM. The combined organic layers were washed with brine, dried over anhydrous Na₂SO₄ and concentrated. The residue was purified by flash column chromatography on silica gel (PE/EA=10:1 to 3:1) to afford 390 mg of the title compound as a yellow oil (15% yield).

### Step b: 2-(Dimethylamino)-1H-imidazole-5-carbaldehyde

n-BuLi (2.5 M, 2.1 mL, 5.37 mmol) was added to a solution of 2-(dimethylamino)-N,N-dimethyl-1H-imidazole-1-sulfonamide (390 mg, 1.79 mmol) in THF (5.0 mL) at −78° C. and the mixture was stirred at −78° C. for 30 min. DMF (0.9 mL, 11.3 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated to give the intermediate. Concentrated HCl (1.0 mL) was added to a solution of the intermediate in THF (5.0 mL) and the reaction mixture was stirred at room temperature for 1 h. The mixture was adjusted to pH=8 with saturated NaHCO₃ solution and extracted with ethyl acetate. The combined extracts were washed with brine, dried over anhydrous Na₂SO₄ and concentrated to afford 100 mg of the title compound as a colorless oil (23% yield over two steps).

### Intermediate 19: 2-(1-(2-Fluoroethyl)piperidin-4-yl)-1H-imidazole-5-carbaldehyde

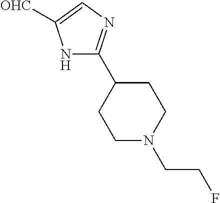 [SMILES: O=Cc1cnc(C2CCN(CCF)CC2)[nH]1]

### Step a: 1-(2-fluoroethyl)piperidine-4-carbonitrile

A mixture of piperidine-4-carbonitrile (4.98 g, 45.2 mmol), 2-fluoroethyl 4-methylbenzenesulfonate (9.86 g, 45.2 mmol) and K₂CO₃ (18.73 g, 135.7 mmol) in DMF (50.0 mL) was stirred at 80° C. overnight. The mixture was cooled to room temperature and extracted with ethyl acetate. The combined extracts were washed with brine, dried over anhydrous Na₂SO₄ and concentrated to afford 6.9 g of the title compound as a white solid (97% yield).

### Step b: ethyl 1-(2-fluoroethyl)piperidine-4-carbimidate hydrochloride

A mixture of 1-(2-fluoroethyl)piperidine-4-carbonitrile (1.96 g, 12.56 mmol) in 40% HCl-EtOH (40.0 mL) was stirred at room temperature overnight. The mixture was concentrated to give a quantitative amount of the title compound as a white solid.

### Step c: 1-(2-fluoroethyl)-4-(1H-imidazol-2-yl)piperidine

A solution of ethyl 1-(2-fluoroethyl)piperidine-4-carbimidate hydrochloride (3.0 g, 14.8 mmol) and aminoacetaldehyde dimethyl acetal (1.75 g, 16.67 mmol) in methanol (20.0 mL) was stirred at room temperature overnight. The mixture was concentrated and concentrated HCl (5.0 mL) was added. The mixture was concentrated again and water (10.0 mL) was added. The mixture was adjusted to pH=11 with K₂CO₃ solution and ethanol (10.0 mL) was added. The resulting mixture was filtered and the filtrate was concentrated. The residue was purified by flash column chromatography on silica gel (DCM/MeOH=100:1 to 20:1) to afford 0.84 g of the title compound as a white solid (41% yield).

### Step d: 2-(1-(2-fluoroethyl)piperidin-4-yl)-N,N-dimethyl-1H-imidazole-1-sulfonamide

1-(2-Fluoroethyl)-4-(1H-imidazol-2-yl)piperidine (800 mg, 4.06 mmol) was added to a suspension of 60% sodium hydride (325 mg, 8.12 mmol) in DMF (6.0 mL) at 0° C. and the mixture was stirred for 0.5 h. Then N,N-dimethylsulfamoyl chloride (638 mg, 4.47 mmol) was added dropwise and the reaction mixture was stirred at room temperature for 1 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (DCM/MeOH=15:1) to afford 920 mg of the title compound as a white solid (74% yield).

### Step e: 2-(1-(2-fluoroethyl)piperidin-4-yl)-1H-imidazole-5-carbaldehyde

n-BuLi (2.5 M, 1.2 mL, 2.75 mmol) was added to a solution of 2-(1-(2-fluoroethyl)piperidin-4-yl)-N,N-dimethyl-1H-imidazole-1-sulfonamide (760 mg, 2.5 mmol) in THF (5.0 mL) at −78° C. and the mixture was stirred at −78° C. for 30 min. DMF (1.4 mL, 17.6 mmol) was added and the reaction mixture was stirred at −78° C. for 1 h and allowed to warm to room temperature over 1 h. Saturated NH₄Cl solution was added and the resulting mixture was extracted with ethyl acetate. The combined extracts were washed with brine, dried over anhydrous Na₂SO₄ and concentrated to give the intermediate (920 mg). Concentrated HCl (1.0 mL) was added to a solution of the intermediate (920 mg, 2.77 mmol) in THF (10.0 mL) and the reaction mixture was stirred at room temperature for 1 h. The mixture was adjusted to pH=8 with saturated NaHCO₃ solution and extracted with ethyl acetate. The combined extracts were washed with brine, dried over anhydrous Na₂SO₄ and concentrated to afford 580 mg of the title compound as a yellow solid (93% yield over two steps).

### Intermediate 20: 5-Methoxyindolin-2-one

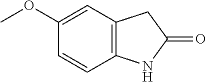

A solution of 5-methoxyisatin (1.0 g, 5.6 mmol) in hydrazine hydrate (85%, 6.0 mL) was refluxed for 3 h. After the mixture was concentrated, the residue was treated with 50% aqueous NaOH solution (40.0 mL) and the resulting mixture was stirred at room temperature for 48 h. The mixture was poured into water and extracted with diethyl ether. The combined extracts were washed with brine, dried over anhydrous Na₂SO₄ and concentrated. The residue was purified by flash column chromatography (PE/EA=10:1 to 2:1) to afford 184 mg of the title compound as a white solid (20% yield).

The following intermediates were similarly prepared from the corresponding isatins:

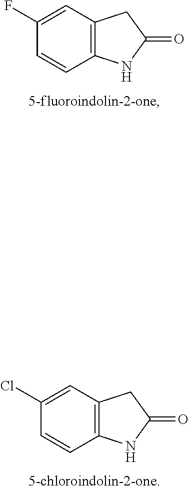 [SMILES: O=C1Cc2cc(F)ccc2N1 ; O=C1Cc2cc(Cl)ccc2N1]

### Intermediate 21: 6-Methoxyindolin-2-one

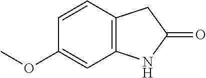

### Step a: diethyl 2-(4-methoxy-2-nitrophenyl)malonate

Diethyl malonate (35.2 g, 0.22 mol) was added dropwise to a suspension of 60% sodium hydride (8.8 g, 0.22 mol) in DMF (150.0 mL) at room temperature. The mixture was heated at 100° C. for 40 min and then cooled to room temperature. 1-Chloro-4-methoxy-2-nitrobenzene (18.7 g, 0.10 mol) was added and the reaction mixture was stirred at room temperature for 0.5 h and heated at 100° C. for another hour. After the mixture was cooled to room temperature, aqueous NH₄Cl solution was added and the resulting mixture was extracted with ethyl acetate. The combined extracts were washed with brine, dried over anhydrous Na₂SO₄ and concentrated. The residue was purified by flash column chromatography on silica gel (DCM/MeOH=30:1 to 20:1) to afford 23.3 g of the title compound (75% yield).

### Step b: ethyl 2-(4-methoxy-2-nitrophenyl)acetate

A mixture of diethyl 2-(4-methoxy-2-nitrophenyl)malonate (9.93 g, 30.0 mmol) and LiOH—H₂O (1.23 g, 30 mmol) in DMSO (50.0 mL) containing water (1.0 mL) was heated at 100° C. for 2 h. The mixture was cooled to room temperature, pounded into water and extracted with ethyl acetate. The combined extracts were washed with brine, dried over anhydrous Na₂SO₄ and concentrated. The residue was purified by flash column chromatography on silica gel (DCM/MeOH=100:1 to 20:1) to afford 4.8 g of the title compound as a yellow oil (76% yield).

### Step c: 6-methoxyindolin-2-one

A mixture of ethyl 2-(4-methoxy-2-nitrophenyl)acetate (2.11 g, 10.0 mmol) and Fe (4.2 g, 75.0 mmol) in AcOH (5.0 mL) was heated at 100° C. for 1 h. The mixture was poured into ice-water and the resulting mixture was extracted with ethyl acetate. The combined extracts were washed with saturated NH₄Cl solution and water, dried over anhydrous Na₂SO₄ and concentrated to afford 700 mg of the title compound as a yellow solid (42% yield).

### Intermediate 22: 5-(Trifluoromethoxy)indolin-2-one

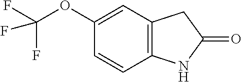 [SMILES: O=C1Cc2cc(OC(F)(F)F)ccc2N1]

### Step a: (E)-2-(hydroxyimino)-N-(4-(trifluoromethoxy)phenyl)acetamide

A mixture of 4-(trifluoromethoxy)aniline (1.0 g, 5.65 mmol) and 2,2,2-trichloroacetaldehyde (1.5 g, 10.18 mmol) in water (10.0 mL) was stirred at room temperature for 1 h. Then hydroxylamine hydrochloride (0.63 g, 9.07 mmol) was added and the reaction mixture was heated at 80° C. for 1.5 h. The mixture was cooled to room temperature and the precipitate was collected by filtration, washed with water and dried under vacuum to give crude title compound, which was used in the next step without further purification.

### Step b: 5-(trifluoromethoxy)indoline-2,3-dione

A solution of crude (E)-2-(hydroxyimino)-N-(4-(trifluoromethoxy)phenyl) acetamide in concentrated H₂SO₄ (5.0 mL) was heated at 70° C. for 5 h. After the mixture was cooled to room temperature, it was poured into ice-water. The resulting mixture was adjusted to pH=7 with saturated Na₂CO₃ solution and extracted with ethyl acetate. The combined extracts were washed with brine, dried over anhydrous Na₂SO₄ and concentrated. The residue was purified by flash column chromatography on silica gel (PE/EA=4:1 to 2:1) to afford 0.4 g of the title compound (29% yield over two steps).

### Step c: 5-(trifluoromethoxy)indolin-2-one

A solution of 5-(trifluoromethoxy)indoline-2,3-dione (0.2 g, 0.87 mmol) in hydrazine hydrate (85%, 2.0 mL) was heated at 130° C. for 4 h. After the mixture was cooled to room temperature, it was poured into ice-water (10.0 mL). The resulting mixture was adjusted to pH=2 and stirred at room temperature for 2 d. The precipitate was collected by filtration, washed with water and dried under vacuum to give 130 mg of the title compound (69% yield).

The following intermediate was prepared in a similar fashion starting from the corresponding aniline:

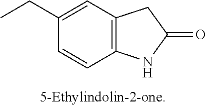

### Intermediate 23: 5-Ethoxyindolin-2-one

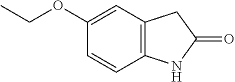

### Step a: diethyl 2-(5-fluoro-2-nitrophenyl)malonate

Diethyl malonate (35.2 g, 0.22 mol) was added dropwise to a suspension of 60% sodium hydride (8.8 g, 0.22 mol) in DMF (150.0 mL) at room temperature. The mixture was heated at 100° C. for 40 min and then cooled to room temperature. 2,4-Difluoronitrobenzene (14.0 g, 0.1 mol) was added and the reaction mixture was stirred at room temperature for 0.5 h and heated at in 100° C. for another hour. After the mixture was cooled to room temperature, aqueous NH₄Cl solution was added and the resulting mixture was extracted with ethyl acetate. The combined extracts were washed with brine, dried over anhydrous Na₂SO₄ and concentrated. The residue was purified by flash column chromatography on silica gel (DCM/MeOH=30:1 to 20:1) to afford 20 g of the title compound (67% yield).

### Step b: ethyl 2-(5-fluoro-2-nitrophenyl)acetate

A mixture of diethyl 2-(5-fluoro-2-nitrophenyl)malonate (20.0 g, 66.9 mmol) and LiOH—H₂O (20.0 g, 66.9 mmol) in DMSO (100.0 mL) containing water (1.2 mL) was heated at 100° C. for 4 h. The mixture was cooled to room temperature, pounded into water and extracted with ethyl acetate. The combined extracts were washed with brine, dried over anhydrous Na₂SO₄ and concentrated. The residue was purified by flash column chromatography on silica gel (DCM/MeOH=100:1 to 20:1) to afford 10 g of the title compound as a yellow oil (66% yield).

### Step c: ethyl 2-(5-ethoxy-2-nitrophenyl)acetate

A mixture of ethyl 2-(5-fluoro-2-nitrophenyl)acetate (0.5 g, 2.2 mmol) and sodium ethanolate (0.45 g, 6.6 mmol) in DMF (20.0 mL) was stirred at room temperature overnight. The mixture was poured into water and extracted with ethyl acetate. The combined extracts were washed with brine, dried over anhydrous Na₂SO₄ and concentrated. The residue was purified by flash column chromatography on silica gel (PE/EA=15:1) to afford 400 mg of the title compound as a colorless oil (36% yield).

### Step d: 5-ethoxyindolin-2-one

A mixture of ethyl 2-(5-ethoxy-2-nitrophenyl)acetate (190 mg, 0.75 mmol) and Fe (168 mg, 3.0 mmol) in AcOH (2.0 mL) was heated at 100° C. for 1 h. The mixture was poured into ice-water and the resulting mixture was extracted with ethyl acetate. The combined extracts were washed with saturated NH₄Cl solution and water, dried over anhydrous Na₂SO₄ and concentrated to afford 80 mg of the title compound as a white solid (60% yield).

### Intermediate 24: 5-(Methylsulfonyl)indolin-2-one

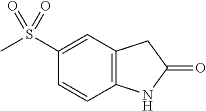

### Step a: ethyl 2-(5-(methylthio)-2-nitrophenyl)acetate

A mixture of ethyl 2-(5-fluoro-2-nitrophenyl)acetate (2.0 g, 8.8 mmol) and sodium thiomethoxide (1.23 g, 17.6 mmol) in DMSO (15.0 mL) was stirred at room temperature overnight. The mixture was poured into water and extracted with ethyl acetate. The combined extracts were washed with brine, dried over anhydrous Na₂SO₄ and concentrated. The residue was purified by flash column chromatography on silica gel (DCM/MeOH=40:1 to 20:1) to afford 400 mg of the title compound as a yellow solid (18% yield).

### Step b: 5-(methylthio)indolin-2-one

A mixture of ethyl 2-(5-(methylthio)-2-nitrophenyl)acetate (400 mg, 1.6 mmol) and Fe (351 mg, 6.4 mmol) in AcOH (4.0 mL) was heated at 100° C. for 1 h. The mixture was poured into ice-water and the resulting mixture was extracted with ethyl acetate. The combined extracts were washed with saturated NH₄Cl solution and water, dried over anhydrous Na₂SO₄ and concentrated to afford 250 mg of the title compound as a grey solid (89% yield).

### Step c: 5-(methylsulfonyl)indolin-2-one

To a solution of 5-(methylthio)indolin-2-one (230 mg, 1.28 mmol) in DCM (10.0 mL) was added m-CPBA (663 mg, 3.85 mmol) at 0° C. The reaction mixture was allowed to warm to room temperature and stirred for 4 h. The mixture was concentrated and the residue was purified by flash column chromatography on silica gel (PE/EA=5:1 to 1:1) to afford 200 mg of the title compound as an orange solid (74% yield).

### Intermediate 25: 5-Isopropoxyindolin-2-one

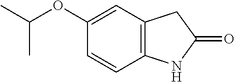

### Step a: 4-isopropoxy-2-methyl-1-nitrobenzene

60% Sodium hydride (0.88 g, 24.0 mmol) was added slowly to a solution of 3-methyl-4-nitrophenol (3.06 g, 20.0 mmol) in DMF (25.0 mL) at 0° C. After the mixture was stirred at 0° C. for 30 min, 2-iodopropane (4.08 g, 24.0 mmol) was added dropwise. The reaction mixture was stirred at 0° C. for 30 min and then allowed to warm to room temperature and stirred overnight. The mixture was poured into water and extracted with ethyl acetate. The combined extracts were washed with brine, dried over anhydrous Na₂SO₄ and concentrated to give 4.3 g of the title compound as an oil.

### Step b: 2-(5-isopropoxy-2-nitrophenyl)acetic acid

Freshly prepared NaOEt solution (0.72 g of Na in 20.0 mL of EtOH) was added dropwise to a solution of 4-isopropoxy-2-methyl-1-nitrobenzene (4.3 g, 20.0 mmol) and diethyl oxalate (3.2 g, 22.0 mmol) in Et₂O (30.0 mL). The reaction mixture was stirred at room temperature overnight and water (50.0 mL) was added. The resulting mixture was adjusted to pH=9-10 with NaOH solution (2.0 N) and stirred at room temperature until 4-isopropoxy-2-methyl-1-nitrobenzene disappeared by TLC. The aqueous phase was separated and H₂O₂ (20.0 mL) was added dropwise at 0° C. The reaction mixture was stirred at 0° C. for 1 h and then adjusted to pH=1-2 with HCl solution (1.0 N). The mixture was extracted with ethyl acetate and the combined organic layers were washed with brine and water, dried over anhydrous Na₂SO₄ and concentrated. The residue was purified by recrystallization from EtOAc/Hexane to afford 1.3 g of the title compound as a light yellow solid (27% yield over three steps).

### Step c: 5-isopropoxyindolin-2-one

A mixture of 2-(5-isopropoxy-2-nitrophenyl)acetic acid (135 mg, 0.56 mmol) and Fe (126 mg, 2.26 mmol) in AcOH (2.0 mL) was heated at 100° C. for 1 h. The mixture was poured into ice-water and the resulting mixture was extracted with ethyl acetate. The combined extracts were washed with saturated NH₄Cl solution and water, dried over anhydrous Na₂SO₄ and concentrated to afford 100 mg of the title compound (93% yield).

### Intermediate 26: 5-Hydroxyindolin-2-one

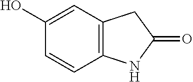 [SMILES: O=C1Cc2cc(O)ccc2N1]

A solution of 5-methoxyindolin-2-one (200 mg, 1.23 mmol) in 40% aqueous HBr (6.0 mL) was heated at 120° C. for 16 h. The mixture was cooled to room temperature and adjusted to pH=4-5 with 20% aqueous NaHCO₃ solution. The precipitate was collected by filtration to give 120 mg of the title compound as a white solid (65% yield).

### Intermediate 27: 5-(2-Fluoroethoxy)indolin-2-one

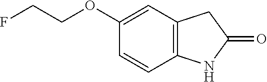 [SMILES: O=C1Cc2cc(OCCF)ccc2N1]

### Step a: 4-(2-fluoroethoxy)-2-methyl-1-nitrobenzene

A solution of 3-methyl-4-nitrophenol (1.33 g, 8.72 mmol), 2-fluoroethyl 4-methylbenzenesulfonate (1.90 g, 8.72 mmol) and K₂CO₃ (3.6 g, 26.1 mmol) in DMF (10.0 mL) was stirred at 80° C. for 3 h. The mixture was pound into water and extracted with ethyl acetate. The combined extracts were washed with brine, dried over anhydrous Na₂SO₄ and concentrated to give 1.57 g of the title compound as a white solid (91% yield).

### Step b: ethyl 3-(5-(2-fluoroethoxy)-2-nitrophenyl)-2-oxopropanoate

NaOEt solution (freshly prepared from 0.33 g of Na in 2.0 mL of EtOH) was added dropwise to a mixture of 4-(2-fluoroethoxy)-2-methyl-1-nitrobenzene (1.02 g, 5.13 mmol) and diethyl oxalate (1.12 g, 7.69 mmol) in Et₂O (10.0 mL) and the reaction mixture was stirred at room temperature overnight. Water was added and the mixture was extracted with ethyl acetate. The combined extracts were washed with brine, dried over anhydrous Na₂SO₄ and concentrated. The residue was purified by flash column chromatography on silica gel (PE/EA=20:1 to 8:1) to afford 920 mg of the title compound as a yellow oil (64% yield).

### Step c: 2-(5-(2-fluoroethoxy)-2-nitrophenyl)acetic acid

A mixture of ethyl 3-(5-(2-fluoroethoxy)-2-nitrophenyl)-2-oxopropanoate (920 mg, 3.08 mmol) in NaOH/water (10.0 mL, pH=9-10) was stirred at room temperature until disappearance of ethyl 3-(5-(2-fluoroethoxy)-2-nitrophenyl)-2-oxopropanoate by TLC. H₂O₂ (20.0 mL) was added dropwise to keep the temperature below 25° C. and the reaction mixture was stirred at room temperature for 1 h. 1 N HCl solution was added to adjust pH=1-2 and the resulting mixture was extracted with ethyl acetate. The combined organic layers were washed with brine, dried over anhydrous Na₂SO₄ and concentrated to afford 710 mg of the title compound as a light yellow solid (95% yield).

### Step d: 5-(2-fluoroethoxy)indolin-2-one

To a solution of 2-(5-(2-fluoroethoxy)-2-nitrophenyl)acetic acid (710 mg, 2.92 mmol) in AcOH (5.0 mL) was added Fe (654 mg, 11.69 mmol) and the reaction mixture was heated at 100° C. for 1 h. After the mixture was cooled to room temperature, it was poured into ice water. The resulting mixture was extracted with ethyl acetate and the combined extracts were washed with brine, dried over anhydrous Na₂SO₄ and concentrated to afford 90 mg of the title compound as a grey white solid (16% yield).

### Intermediate 28: 7-fluoro-5-isopropoxyindolin-2-one

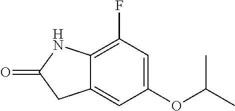

### Step a: 3,5-difluoro-4-nitrophenol

To a solution of 3,5-difluorophenol (26.0 g, 200 mmol) in DCM (500 mL) at 0° C. was added 70% HNO₃ (14 mL, 200.0 mmol) dropwise. The reaction mixture was allowed to warm to room temperature and stirred at for 30 min. The precipitate was collected by filtration, washed with water and dried under vacuum to give 15.2 g of the title compound as a yellow solid (43% yield).

### Step b: 1,3-difluoro-5-isopropoxy-2-nitrobenzene

2-Iodopropane (12.6 g, 74.2 mmol) was added to a mixture of 3,5-difluoro-4-nitrophenol (11.8 g, 67.4 mmol) and K₂CO₃ (18.6 g, 134.9 mmol) in DMF (100 mL) at room temperature. The reaction mixture was stirred at 60° C. for 12 h. After the mixture was cooled to room temperature, it was poured into water and the resulting mixture was extracted with ethyl acetate. The combined extracts were washed with brine, dried over anhydrous Na₂SO₄ and concentrated to afford 6.1 g of the title compound as a yellow oil (42% yield).

### Step c: diethyl 2-(3-fluoro-5-isopropoxy-2-nitrophenyl)malonate

NaH (60% suspension, 552 mg, 13.82 mmol) was added to a mixture of diethyl malonate (2.2 g, 13.82 mmol) in DMSO (15.0 mL) at room temperature carefully and the mixture was heated at 100° C. for 4 h. After the mixture was cooled to room temperature, 1,3-difluoro-5-isopropoxy-2-nitrobenzene (2 g, 9.22 mmol) was added. The reaction mixture was stirred at room temperature for 30 min and then heated at 100° C. for 1 h. Water was added and the resulting mixture was extracted with ethyl acetate. The combined extracts were washed with brine, dried over anhydrous Na₂SO₄ and concentrated to afford 2.8 g of the title compound as a yellow solid (85% yield).

### Step d: ethyl 2-(3-fluoro-5-isopropoxy-2-nitrophenyl)acetate

A mixture of diethyl 2-(3-fluoro-5-isopropoxy-2-nitrophenyl)malonate (1.64 g, 6.7 mmol), LiCl (392 mg, 9.22 mmol) and water (0.08 mL, 4.6 mmol) in DMSO (20 mL) was heated at 100° C. overnight. After the mixture was cooled to room temperature, water was added and the resulting mixture was extracted with ethyl acetate. The combined extracts were washed with brine and water, dried over anhydrous Na₂SO₄ and concentrated to afford 1.13 g of the title compound as a light yellow solid (87% yield).

### Step e: 7-fluoro-5-isopropoxyindolin-2-one

Ethyl 2-(3-fluoro-5-isopropoxy-2-nitrophenyl)acetate (1.31 g, 4.61 mmol) was dissolved in AcOH (10.0 mL) and Fe (1.03 g, 18.44 mmol) was added. The reaction mixture was heated at 100° C. for 1 h. After the mixture was cooled to room temperature, it was poured into ice water and the resulting mixture was extracted with ethyl acetate. The combined extracts were washed with saturated NH₄Cl solution and water, dried over anhydrous Na₂SO₄ and concentrated. The residue was washed with Et₂O to afford 180 mg of the title compound as a grey solid (20% yield).

### Intermediate 29: 6-fluoro-5-isopropoxyindolin-2-one

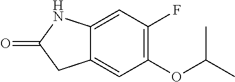

### Step a: 2,5-difluoro-4-nitrophenol

To a solution of 2,5-difluorophenol (13.0 g, 100 mmol) in DCM (200.0 mL) at 0° C. was added 70% HNO₃ (7.0 mL, 100.0 mmol) dropwise. The reaction mixture was allowed to warm to room temperature and stirred at for 30 min. The precipitate was collected by filtration, washed with water and dried under vacuum to give 5.6 g of the title compound as a yellow solid (32% yield).

### Step b: 1,4-difluoro-2-isopropoxy-5-nitrobenzene

2-Iodopropane (4.08 g, 24 mmol) was added to a mixture of 2,5-difluoro-4-nitrophenol (3.5 g, 20 mmol) and K₂CO₃ (5.52 g, 40 mmol) in DMF (35 mL) at room temperature. The reaction mixture was stirred at 70° C. for 12 h. After the mixture was cooled to room temperature, it was poured into water and the resulting mixture was extracted with ethyl acetate. The combined extracts were washed with brine, dried over anhydrous Na₂SO₄ and concentrated. The residue was purified by flash column chromatography on silica gel (PE/EA=20:1 to 8:1) to afford 2.2 g of the title compound as a yellow solid (50% yield).

### Step c: diethyl 2-(4-fluoro-5-isopropoxy-2-nitrophenyl)malonate

NaH (60% suspension, 0.37 g, 9.22 mmol) was added to a mixture of diethyl malonate (1.48 g, 9.22 mmol) in DMSO (15.0 mL) at room temperature carefully and the mixture was heated at 100° C. for 4 h. After the mixture was cooled to room temperature, 1,4-difluoro-2-isopropoxy-5-nitrobenzene (2 g, 9.22 mmol) was added. The reaction mixture was stirred at room temperature for 30 min and then heated at 100° C. for 1 h. Water was added and the resulting mixture was extracted with ethyl acetate. The combined extracts were washed with brine, dried over anhydrous Na₂SO₄ and concentrated to afford 2.83 g of the title compound as a yellow solid (86% yield).

### Step d: ethyl 2-(4-fluoro-5-isopropoxy-2-nitrophenyl)acetate

A mixture of diethyl 2-(4-fluoro-5-isopropoxy-2-nitrophenyl)malonate (2.4 g, 6.7 mmol), LiCl (571 mg, 13.4 mmol) and water (0.12 mL, 6.7 mmol) in DMSO (20 mL) was heated at 100° C. overnight. After the mixture was cooled to room temperature, water was added and the resulting mixture was extracted with ethyl acetate. The combined extracts were washed with brine and water, dried over anhydrous Na₂SO₄ and concentrated to afford 1.1 g of the title compound as a light yellow oil (58% yield).

### Step e: 6-fluoro-5-isopropoxyindolin-2-one

Ethyl 2-(4-fluoro-5-isopropoxy-2-nitrophenyl)acetate (1.1 g, 3.86 mmol) was dissolved in AcOH (10.0 mL) and Fe (865 mg, 15.44 mmol) was added. The reaction mixture was heated at 100° C. for 1 h. After the mixture was cooled to room temperature, it was poured into ice water and the resulting mixture was extracted with ethyl acetate. The combined extracts were washed with saturated NH₄Cl solution and water, dried over anhydrous Na₂SO₄ and concentrated. The residue was washed with Et₂O to afford 300 mg of the title compound as a grey solid (37% yield).

### Intermediate 30: 4-fluoro-5-isopropoxyindolin-2-one

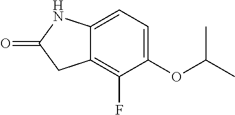

### Step a: 2,3-difluoro-4-nitrophenol

To a solution of 2,3-difluorophenol (52.0 g, 400.0 mmol) in DCM (500.0 mL) at 0° C. was added 70% HNO₃ (28.0 mL, 400.0 mmol) dropwise. The reaction mixture was allowed to warm to room temperature and stirred at for 30 min. The precipitate was collected by filtration, washed with water and dried under vacuum to give 20 g of the title compound as a white solid (29% yield).

### Step b: 2,3-difluoro-1-isopropoxy-4-nitrobenzene

2-Iodopropane (7.3 g, 42.9 mmol) was added to a mixture of 2,3-difluoro-4-nitrophenol (5.0 g, 28.6 mmol) and K₂CO₃ (8.0 g, 57.0 mmol) in DMSO (20.0 mL) at room temperature. The reaction mixture was stirred at 50° C. for 8 h. After the mixture was cooled to room temperature, it was poured into water and the resulting mixture was extracted with ethyl acetate. The combined extracts were washed with brine, dried over anhydrous Na₂SO₄ and concentrated to afford 3.0 g of the title compound as a yellow solid (48% yield).

### Step c: diethyl 2-(2-fluoro-3-isopropoxy-6-nitrophenyl)malonate

NaH (60% suspension, 0.6 g, 15.0 mmol) was added to a mixture of diethyl malonate (2.4 g, 15.0 mmol) in DMSO (15.0 mL) at room temperature carefully and the mixture was heated at 100° C. for 4 h. After the mixture was cooled to room temperature, 2,3-difluoro-1-isopropoxy-4-nitrobenzene (2.17 g, 10.0 mmol) was added. The reaction mixture was stirred at room temperature for 30 min and then heated at 100° C. for 1 h. Water was added and the resulting mixture was extracted with ethyl acetate. The combined extracts were washed with brine, dried over anhydrous Na₂SO₄ and concentrated. The residue was purified by flash column chromatography on silica gel (PE/EA=20:1) to afford 1.5 g of the title compound as a light yellow solid (42% yield).

### Step d: ethyl 2-(2-fluoro-3-isopropoxy-6-nitrophenyl)acetate

A mixture of diethyl 2-(2-fluoro-3-isopropoxy-6-nitrophenyl)malonate (500 mg, 1.4 mmol), LiCl (120 mg, 2.8 mmol) and water (0.03 mL, 1.4 mmol) in DMSO (5.0 mL) was heated at 100° C. overnight. After the mixture was cooled to room temperature, water was added and the resulting mixture was extracted with ethyl acetate. The combined extracts were washed with brine and water, dried over anhydrous Na₂SO₄ and concentrated to afford 400 mg of the title compound as a light yellow oil.

### Step e: 4-fluoro-5-isopropoxyindolin-2-one

Ethyl 2-(2-fluoro-3-isopropoxy-6-nitrophenyl)acetate (400 mg, 1.4 mmol) was dissolved in AcOH (10.0 mL) and Fe (314 mg, 5.6 mmol) was added. The reaction mixture was heated at 100° C. for 1 h. After the mixture was cooled to room temperature, it was poured into ice water and the resulting mixture was extracted with ethyl acetate. The combined extracts were washed with saturated NH₄Cl solution and water, dried over anhydrous Na₂SO₄ and concentrated. The residue was washed with Et₂O to afford 60 mg of the title compound as a grey solid (21% yield over two steps).

### Intermediate 31: 5-tetrahydropyran-4-yl-1H-imidazole-2-carbaldehyde

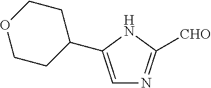 [SMILES: O=Cc1ncc(C2CCOCC2)[nH]1]

### Step a: 4,5-diiodo-1H-imidazole

A mixture of KI (149.4 g, 0.91 mol) and I₂ (151.0 g, 0.59 mol) in water (500 mL) was added to a mixture of 1H-imidazole (20.0 g, 0.29 mol) in 2N NaOH (1.0 L). The reaction mixture was stirred at room temperature for 2 d. The mixture was neutralized with 6N HCl (pH=7) and the resulting solid was collected by filtration, washed with water and dried under vacuum to give 80.0 g of the title compound as a grey solid (85% yield).

### Step b: 4-(4-iodo-1H-imidazol-5-yl)tetrahydro-2H-pyran-4-ol

n-BuLi (2.5 M, 24.8 mL, 62.0 mmol) was added to a solution of 4,5-diiodo-1H-imidazole (9.6 g, 30.0 mmol) in THF (100.0 mL) at −78° C. and the mixture was stirred at −78° C. for 30 min. Dihydro-2H-pyran-4(3H)-one (6.0 g, 60.0 mmol) was added and the reaction mixture was stirred at −78° C. for 10 min and then allowed to warm to room temperature over 1 h. Saturated NH4Cl solution was added and the resulting mixture was extracted with ethyl acetate. The combined extracts were washed with brine, dried over anhydrous Na₂SO₄ and concentrated. The residue was purified by flash column chromatography on silica gel (PE to PE/EA=1:1 to DCM/MeOH=50:1) to give 2.7 g of the title compound as a white solid (30% yield).

### Step c: 5-(3,6-dihydro-2H-pyran-4-yl)-1H-imidazole

A mixture of 4-(4-iodo-1H-imidazol-5-yl)tetrahydro-2H-pyran-4-ol (2.7 g, 9.2 mmol) in 4 N HCl (20.0 mL) was heated at 30° C. for 12 h. The mixture was concentrated under reduced pressure to give 0.8 g of the title compound as a white solid (64% yield).

### Step d: 5-(3,6-dihydro-2H-pyran-4-yl)-N,N-dimethyl-1H-imidazole-1-sulfonamide

NaH (60% suspension, 0.28 g, 6.9 mmol) was added to a mixture of 5-(3,6-dihydro-2H-pyran-4-yl)-1H-imidazole (0.8 g, 5.3 mmol) in DMF (10.0 mL) and the mixture was stirred at room temperature for 30 min. DMSCl (0.91 g, 6.4 mmol) was added and the reaction mixture was stirred at room temperature for 2 h. Water was added and the resulting mixture was extracted with ethyl acetate. The combined extracts were washed with brine, dried over anhydrous Na₂SO₄ and concentrated. The residue was purified by flash column chromatography on silica gel (PE to PE/EA=5:1 to 1:1) to give 0.71 g of the title compound as a white solid (52% yield).

### Step e: N,N-dimethyl-5-(tetrahydro-2H-pyran-4-yl)-1H-imidazole-1-sulfonamide

To a solution of 5-(3,6-dihydro-2H-pyran-4-yl)-N,N-dimethyl-1H-imidazole-1-sulfonamide (0.33 g, 1.28 mmol) in EtOH (100.0 mL) was added 10% Pd/C (33 mg) and the resulting mixture was degassed. The reaction mixture was stirred under H₂ atmosphere (20 atm) at 65° C. for 5 h. The catalyst was filtered off and the filtrate was concentrated under reduced pressure. The residue was purified by flash column chromatography on silica gel (PE/EA=4/1 to 2/1) to give 140 mg of the title compound as a light yellow solid (42% yield).

### Step f: 5-tetrahydropyran-4-yl-1H-imidazole-2-carbaldehyde

n-BuLi (2.5 M, 0.6 mL, 1.5 mmol) was added to a solution of N,N-dimethyl-5-(tetrahydro-2H-pyran-4-yl)-1H-imidazole-1-sulfonamide (0.3 g, 1.15 mmol) in THF (10.0 mL) at −78° C. and the mixture was stirred at −78° C. for 30 min. DMF (0.7 mL, 6.9 mmol) was added and the reaction mixture was stirred at −78° C. for 1 h and allowed to warm to room temperature over 1 h. The mixture was adjusted to pH=1 with concentrated HCl and stirred for 2 h. Then the mixture was adjusted to pH=8 with aqueous NaHCO₃ solution and extracted with ethyl acetate. The combined extracts were washed with brine, dried over anhydrous Na₂SO₄ and concentrated. The residue was purified by flash column chromatography on silica gel (PE/EA=1:1) to give 80 mg of the title compound as a grey white solid (40% yield).

### Intermediate 32: 1-(2-morpholino-1H-imidazol-5-yl)ethanone

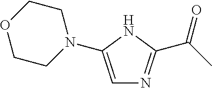

### Step a: 5-acetyl-N,N-dimethyl-2-morpholino-1H-imidazole-1-sulfonamide

n-BuLi (2.5 M, 4.2 mL, 10.5 mmol) was added to a solution of N,N-dimethyl-2-morpholino-1H-imidazole-1-sulfonamide (2.2 g, 8.5 mmol) in THF (20.0 mL) at −78° C. and the mixture was stirred at −78° C. for 30 min. Ac₂O (1.5 mL) was added and the reaction mixture was stirred at −78° C. for 10 min and allowed to warm to room temperature over 1 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (PE/EA=5:1 to 2:1) to afford 0.4 g of the title compound as an oil (16% yield).

### Step b: 1-(2-morpholino-1H-imidazol-5-yl)ethanone

Concentrated HCl (1.0 mL) was added to a solution of 5-acetyl-N,N-dimethyl-2-morpholino-1H-imidazole-1-sulfonamide (0.4 g, 0.32 mmol) in THF (20.0 mL) and water (3.0 mL) and the reaction mixture was heated at 50-60° C. for 2 h. After the mixture was cooled to room temperature, it was adjusted to pH=7 with saturated Na₂CO₃ solution and extracted with ethyl acetate. The combined extracts were washed with brine, dried over anhydrous Na₂SO₄ and concentrated. The residue was purified by flash column chromatography on silica gel (PE/EA=2:1 to 1:1) to afford 150 mg of the title compound as a yellow solid (58% yield).

### Intermediate 33: 2-(4-ethylpiperazin-1-yl)-1H-imidazole-5-carbaldehyde

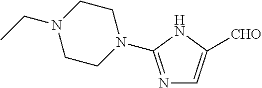

### Step a: N,N-dimethyl-2-(piperazin-1-yl)-1H-imidazole-1-sulfonamide

A mixture of 2-bromo-N,N-dimethyl-1H-imidazole-1-sulfonamide (2.54 g, 10.0 mmol) and piperazine (8.6 g, 100 mmol) in 1,4-dioxane (20.0 mL) was heated at 90° C. for 5 h. The mixture was concentrated under reduced pressure and the residue was purified by flash column chromatography on silica gel (PE/EA=1:1 to DCM/MeOH=100:1) to afford 2.5 g of the title compound as a yellow oil (96% yield).

### Step b: 2-(4-ethylpiperazin-1-yl)-N,N-dimethyl-1H-imidazole-1-sulfonamide

NaH (60% suspension, 92 mg, 2.3 mmol) was added to a solution of N,N-dimethyl-2-(piperazin-1-yl)-1H-imidazole-1-sulfonamide (500 mg, 1.9 mmol) in DMF (10.0 mL) and the mixture was stirred at room temperature for 30 min. Iodoethane (296 mg, 1.9 mmol) was added and the reaction mixture was stirred at room temperature for another 3 h. Water was added and the resulting mixture was extracted with ethyl acetate. The combined extracts were washed with brine, dried over anhydrous Na₂SO₄ and concentrated. The residue was purified by flash column chromatography on silica gel (DCM/MeOH=120:1) to afford 287 mg of the title compound as a white solid (52% yield).

### Step c: 2-(4-ethylpiperazin-1-yl)-1H-imidazole-5-carbaldehyde

n-BuLi (2.5 M, 1.0 mL, 2.4 mmol) was added to a solution of 2-(4-ethylpiperazin-1-yl)-N,N-dimethyl-1H-imidazole-1-sulfonamide (574 mg, 2.0 mmol) in THF (10.0 mL) at −78° C. and the mixture was stirred at −78° C. for 30 min. DMF (0.08 mL, 12.0 mmol) was added and the reaction mixture was stirred at −78° C. for 10 min and allowed to warm to room temperature over 1 h. Saturated Na₂CO₃ solution was added and the resulting mixture was extracted with ethyl acetate. The combined extracts were washed with brine, dried over anhydrous Na₂SO₄ and concentrated. The resulting residue was dissolved in concentrated HCl (1.0 mL) and heated at 60° C. for 2 h. The mixture was cooled to room temperature, adjusted to pH=7 with saturated Na₂CO₃ solution and extracted with ethyl acetate. The combined extracts were washed with brine, dried over anhydrous Na₂SO₄ and concentrated to afford 120 mg of the title compound (32% yield).

### Intermediate 34: 5-isopropoxy-1H-pyrrolo[2,3-c]pyridin-2(3H)-one

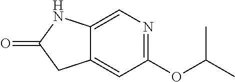

### Step a: 4-chloro-2-isopropoxy-5-nitropyridine

To a solution of 4-chloro-5-nitropyridin-2-ol (4.0 g, 23.0 mmol) in DMSO (25.0 mL) was added K₂CO₃ (6.35 g, 46.0 mmol) and the mixture was stirred at room temperature for 30 min. 2-iodopropane (5.87 g, 34.5 mmol) was added dropwise and the reaction mixture was stirred at 50° C. for 2 h. The mixture was poured into water and the resulting mixture was extracted with ethyl acetate. The combined extracts were washed with brine, dried over anhydrous Na₂SO₄ and concentrated. The residue was purified by flash column chromatography to give 0.9 g of the title compound as an oil (18% yield).

### Step b: diethyl 2-(2-isopropoxy-5-nitropyridin-4-yl)malonate

NaH (60%, 155 mg, 3.9 mmol) was added to a mixture of diethyl malonate (0.58 g, 3.6 mmol) in DMSO (5.0 mL) at room temperature carefully and the mixture was heated at 100° C. for 40 min. After the mixture was cooled to room temperature, 4-chloro-2-isopropoxy-5-nitropyridine (0.7 g, 3.3 mmol) was added. The reaction mixture was stirred at room temperature for 30 min and then heated at 100° C. for 1 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (PE/EA=75:1 to 50:1) to afford 0.6 g of the title compound (53% yield).

### Step c: ethyl 2-(2-isopropoxy-5-nitropyridin-4-yl)acetate

A mixture of diethyl 2-(2-isopropoxy-5-nitropyridin-4-yl)malonate (0.2 g, 0.588 mmol), LiCl (50 mg, 1.176 mmol) and water (1.0 mL) in DMSO (5.0 mL) was heated at 100° C. for 1 h. After the mixture was cooled to room temperature, water was added and the resulting mixture was extracted with ethyl acetate. The combined extracts were washed with brine and water, dried over anhydrous Na₂SO₄ and concentrated to afford 0.18 g of the title compound as a yellow oil (87% yield).

### Step d: 5-isopropoxy-1H-pyrrolo[2,3-c]pyridin-2(3H)-one

Ethyl 2-(2-isopropoxy-5-nitropyridin-4-yl)acetate (0.18 g, 0.67 mmol) was dissolved in AcOH (5.0 mL) and Fe (50 mg, 2.69 mmol) was added. The reaction mixture was heated at 100° C. for 1.5 h. After the mixture was cooled to room temperature, it was poured into ice water and the resulting mixture was extracted with ethyl acetate. The combined extracts were washed with saturated NH₄Cl solution and water, dried over anhydrous Na₂SO₄ and concentrated to afford 88 mg of the title compound (69% yield).

### Intermediate 35: 5-isopropoxy-1H-pyrrolo[3,2-b]pyridin-2(3H)-one

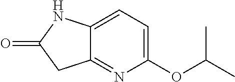

### Step a: 2-bromo-6-isopropoxypyridine

To a solution of 6-bromopyridin-2-ol (9.0 g, 51.7 mmol) in DMF (60.0 mL) was added K₂CO₃ (14.3 g, 103.4 mmol) at 0° C. and the mixture was stirred at 0° C. for 30 min. 2-Iodopropane (10.55 g, 62.0 mmol) was added dropwise and the reaction mixture was stirred at 0° C. for 30 min and then allowed to warm to room temperature and stirred overnight. The mixture was poured into water and the resulting mixture was extracted with ethyl acetate. The combined extracts were washed with brine, dried over anhydrous Na₂SO₄ and concentrated to give 10.0 g of the title compound as an oil (89% yield).

### Step b: 2-bromo-6-isopropoxy-3-nitropyridine

2-Bromo-6-isopropoxypyridine (2.16 g, 10.0 mmol) was added slowly to a mixture of concentrated H₂SO₄ and HNO₃ (2:1, 24.0 mL) and the reaction mixture was stirred at room temperature for 3 h. The mixture was poured into ice-water and the resulting precipitate was collected by filtration, washed with water and dried under vacuum to afford 0.7 g of the title compound as a yellow solid (27% yield).

### Step c: diethyl 2-(6-isopropoxy-3-nitropyridin-2-yl)malonate

NaH (60% suspension, 200 mg, 4.6 mmol) was added to a mixture of diethyl malonate (670 mg, 4.2 mmol) in DMSO (20.0 mL) at room temperature carefully and the mixture was heated at 100° C. for 40 min. After the mixture was cooled to room temperature, 2-bromo-6-isopropoxy-3-nitropyridine (1.0 g, 3.8 mmol) was added. The reaction mixture was stirred at room temperature for 30 min and then heated at 100° C. for 1 h. Saturated NH₄Cl solution was added and the resulting mixture was extracted with ethyl acetate. The combined extracts were washed with brine, dried over anhydrous Na₂SO₄ and concentrated. The residue was purified by flash column chromatography on silica gel (PE/EA=75:1 to 50:1) to afford 0.5 g of the title compound (38% yield).

### Step d: ethyl 2-(6-isopropoxy-3-nitropyridin-2-yl)acetate

A mixture of diethyl 2-(6-isopropoxy-3-nitropyridin-2-yl)malonate (520 m g, 1.53 mmol), LiCl (127 mg, 3.0 mmol) and water (1.0 mL) in DMSO (5.0 mL) was heated at 100° C. for 1 h. After the mixture was cooled to room temperature, water was added and the resulting mixture was extracted with ethyl acetate. The combined extracts were washed with brine and water, dried over anhydrous Na₂SO₄ and concentrated to afford 120 mg of the title compound as a yellow oil (30% yield).

### Step e: 5-isopropoxy-1H-pyrrolo[3,2-b]pyridin-2(3H)-one

Ethyl 2-(6-isopropoxy-3-nitropyridin-2-yl)acetate (120 mg, 0.447 mmol) was dissolved in AcOH (3.0 mL) and Fe (40 mg, 2.0 mmol) was added. The reaction mixture was heated at 100° C. for 1 h. After the mixture was cooled to room temperature, it was poured into ice water and the resulting mixture was extracted with ethyl acetate. The combined extracts were washed with saturated NH₄Cl solution and water, dried over anhydrous Na₂SO₄ and concentrated to afford 80 mg of the title compound as a green solid (91% yield).

### Example 2

### Synthetic Methods

### Method A

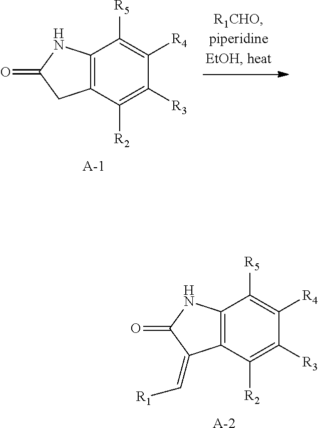

The appropriately substituted indolinone A-1 is heated to reflux in an alcoholic solvent, such as methanol, ethanol, or butanol, in the presence of piperidine and an aldehyde for several hours to give the final compound A-2.

### Example 2-A

### Synthesis of (3Z)-5-Fluoro-3-((2-methyl-1H-imidazol-5-yl)methylene)indolin-2-one

A mixture of 2-methyl-1H-imidazole-5-carbaldehyde (44 mg, 0.40 mmol) and 5-fluoroindolin-2-one (50 mg, 0.33 mmol) in ethanol (2.0 mL) containing piperidine (0.1 mL) was stirred at 90° C. for 3 h. After the mixture was cooled to room temperature, the precipitate was collected by filtration, washed with cold ethanol and dried to afford 28 mg of the title compound (35% yield) as a yellow solid.

### Example 2-B

### Synthesis of (3Z)-5-Isopropoxy-3-((2-methyl-1H-imidazol-5-yl)methylene)indolin-2-one

A mixture of 5-isopropoxyindolin-2-one (103 mg, 0.54 mmol) and 2-methyl-1H-imidazole-5-carbaldehyde (56 mg, 0.51 mmol) in ethanol (2.0 mL) containing piperidine (0.1 mL) was heated at 90° C. for 3 h. After the mixture was cooled to room temperature, the precipitate was collected by filtration, washed with cold ethanol and dried under vacuum to afford 60 mg of the title compound as a yellow solid (41% yield).

### Example 2-C

### Synthesis of (3Z)-5-methoxy-3-[(2-tetrahydropyran-4-yl-1H-imidazol-5-yl)methylene]indolin-2-one

A mixture of 2-(tetrahydro-2H-pyran-4-yl)-1H-imidazole-5-carbaldehyde (100 mg, 0.56 mmol) and 5-methoxyindolin-2-one (95 mg, 0.6 mmol) in ethanol (5.0 mL) containing piperidine (0.1 mL) was heated at 90° C. for 3 h. After the mixture was cooled to room temperature, the precipitate was collected by filtration, washed with cold ethanol and dried under vacuum. The residue was purified by flash column chromatography on silica gel (DCM/MeOH=100:1 to 50:1) to provide 85 mg of the title compounds as an orange solid (46% yield).

The following compounds were prepared in a similar fashion:
(3Z)-3-[(3-methyl-1H-pyrazol-5-yl)methylene]indolin-2-one
(3Z)-5-methoxy-3-[(2-methyl-1H-imidazol-5-yl)methylene]indolin-2-one
(3Z)-3-(1H-pyrrolo[2,3-b]pyridin-2-ylmethylene)indolin-2-one
(3Z)-6-methoxy-3-[(2-methyl-1H-imidazol-5-yl)methylene]indolin-2-one
(3E)-3-(1,3-benzothiazol-2-ylmethylene)indolin-2-one
(3E)-3-(3-quinolylmethylene)indolin-2-one
(3E)-3-(1,3-benzoxazol-2-ylmethylene)indolin-2-one
(3Z)-5-chloro-3-[(2-methyl-1H-imidazol-5-yl)methylene]indolin-2-one
(3Z)-3-[(2-methyl-1H-imidazol-5-yl)methylene]-5-(trifluoromethoxy)indolin-2-one
(3Z)-3-[(2-isopropyl-1H-imidazol-5-yl)methylene]-5-methoxy-indolin-2-one
(3Z)-5-ethoxy-3-[(2-methyl-1H-imidazol-5-yl)methylene]indolin-2-one
(3Z)-3-[(2-methyl-1H-imidazol-5-yl)methylene]-5-methylsulfonyl-indolin-2-one
(3Z)-3-[(2-ethyl-1H-imidazol-5-yl)methylene]-5-methoxy-indolin-2-one
(3Z)-5-methoxy-3-[[2-(methoxymethyl)-1H-imidazol-5-yl]methylene]indolin-2-one
(3Z)-3-[(2-tert-butyl-1H-imidazol-5-yl)methylene]-5-methoxy-indolin-2-one
(3Z)-3-[[2-(2,6-difluorophenyl)-1H-imidazol-5-yl]methylene]-5-methoxy-indolin-2-one
(3Z)-5-isopropoxy-3-[(2-isopropyl-1H-imidazol-5-yl)methylene]indolin-2-one
(3Z)-3-[(2-isobutyl-1H-imidazol-5-yl)methylene]-5-isopropoxy-indolin-2-one
(3Z)-5-isopropoxy-3-[[2-(morpholinomethyl)-1H-imidazol-5-yl]methylene]indolin-2-one
(3Z)-5-methoxy-3-[[2-(2-methoxyethyl)-1H-imidazol-5-yl]methylene]indolin-2-one
(3Z)-5-isopropoxy-3-[(5-isopropyl-4H-1,2,4-triazol-3-yl)methylene]indolin-2-one
(3Z)-5-isopropoxy-3-[(2-morpholino-1H-imidazol-5-yl)methylene]indolin-2-one
(3Z)-5-hydroxy-3-[(2-tetrahydropyran-4-yl-1H-imidazol-5-yl)methylene]indolin-2-one
(3Z)-5-ethyl-3-[(2-tetrahydropyran-4-yl-1H-imidazol-5-yl)methylene]indolin-2-one
(3Z)-5-isopropoxy-3-[(2-tetrahydropyran-4-yl-1H-imidazol-5-yl)methylene]indolin-2-one
(3Z)-3-[[2-[1-(2-fluoroethyl)-4-piperidyl]-1H-imidazol-5-yl]methylene]-5-isopropoxy-indolin-2-one
(3Z)-5-chloro-3-[(2-tetrahydropyran-4-yl-1H-imidazol-5-yl)methylene]indolin-2-one
(3Z)-5-chloro-3-[[2-[1-(2-fluoroethyl)-4-piperidyl]-1H-imidazol-5-yl]methylene]indolin-2-one
(3Z)-5-(2-fluoroethoxy)-3-[(2-tetrahydropyran-4-yl-1H-imidazol-5-yl)methylene]indolin-2-one
(3Z)-3-[[2-(dimethylamino)-1H-imidazol-5-yl]methylene]-5-isopropoxy-indolin-2-one
(3Z)-7-fluoro-5-isopropoxy-3-[(2-morpholino-1H-imidazol-5-yl)methylene]indolin-2-one
(3Z)-6-fluoro-5-isopropoxy-3-[(2-morpholino-1H-imidazol-5-yl)methylene]indolin-2-one
(3Z)-4-fluoro-5-isopropoxy-3-[(2-morpholino-1H-imidazol-5-yl)methylene]indolin-2-one
(3Z)-5-isopropoxy-3-[(5-tetrahydropyran-4-yl-1H-imidazol-2-yl)methylene]indolin-2-one
(3Z)-5-isopropoxy-3-[1-(2-morpholino-1H-imidazol-5-yl)ethylidene]indolin-2-one
(3Z)-6-fluoro-5-isopropoxy-3-[(2-tetrahydropyran-4-yl-1H-imidazol-5-yl)methylene]indolin-2-one
(3Z)-6-fluoro-5-isopropoxy-3-[1-(2-morpholino-1H-imidazol-5-yl)ethylidene]indolin-2-one
(3Z)-6-fluoro-3-[[2-[1-(2-fluoroethyl)-4-piperidyl]-1H-imidazol-5-yl]methylene]-5-isopropoxy-indolin-2-one
(3Z)-3-[[2-(4-ethylpiperazin-1-yl)-1H-imidazol-5-yl]methylene]-6-fluoro-5-isopropoxy-indolin-2-one.

### Method B

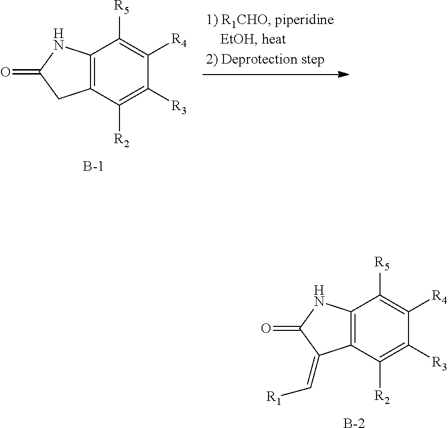

The appropriately substituted indolinone B-1 is heated to reflux in an alcoholic solvent, such as methanol, ethanol, or butanol, in the presence of piperidine and a suitably protected aldehyde for several hours. The isolated material is then submitted to a deprotection step, such as a solution of HCl in a suitable solvent, to provide final compound B-2.

### Example 2-D

### Synthesis of (3Z)-3-[(5-ethyl-4H-1,2,4-triazol-3-yl)methylene]-5-methoxy-indolin-2-one

A mixture of 3-ethyl-5-formyl-N,N-dimethyl-4H-1,2,4-triazole-4-sulfonamide (170 mg, 0.73 mmol) and 5-methoxyindolin-2-one (100 mg, 0.52 mmol) in ethanol (30 mL) containing piperidine (0.1 mL) was heated at 90° C. for 3 h. The mixture was cooled to room temperature and concentrated. The residue was dissolved in THF (20.0 mL) and concentrated HCl (4.0 mL) was added and the reaction mixture was stirred at room temperature for 0.5 h. The mixture was adjusted to pH=7-8 with saturated NaHCO₃ solution and extracted with DCM. The combined organic layers were washed with brine, dried over anhydrous Na₂SO₄ and concentrated. The residue was purified by flash column chromatography on silica gel to afford 20 mg of the title compound as a red solid (10% yield).

### Example 2-E

### Synthesis of (3Z)-3-[(5-methyl-1H-imidazol-2-yl)methylene]indolin-2-one

A mixture of 4-methyl-1-trityl-1H-imidazole-2-carbaldehyde (132 mg, 0.38 mmol) and indolin-2-one (50 mg, 0.38 mmol) in ethanol (2.0 mL) containing piperidine (0.1 mL) was stirred at 90° C. for 3 h. After the mixture was cooled to room temperature, the precipitate was collected by filtration, washed with cold ethanol and dried to afford 120 mg of a yellow solid. The solid was stirred in HCl-EtOH (5.0 M, 5.0 mL) at 0° C. overnight. The reaction mixture was concentrated and the residue was dissolved in ethanol (15.0 mL) containing piperidine (0.2 mL). The solution was heated at 90° C. for 3 h and then cooled to room temperature and poured into water. The resulting solid was collected by filtration, washed with Et₂O and dried to afford 20 mg of the title compound as a brown solid (35% yield).

Table 1 shows analytical data for certain compounds of Formula (I) prepared using procedures described above.

TABLE 1

| | | ¹H NMR | [M + H⁺] |
|---|---|---|---|
| Compound | Structure | (δ, ppm) | (m/z) |
| 1 | 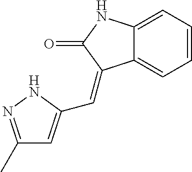 [SMILES: Cc1cc(/C=C2\C(=O)Nc3ccccc32)[nH]n1] | Two tautomers (DMSO-d6, 300 MHz) 2.25 (s, 3H), 6.65-7.23 (m, 4H), 7.68 (m, 2H), 10.6 (s, 0.3H), 11.1 (s, 0.7H), 13.1 (s, 0.3H), 13.9 (s, 0.7H) | 226 |
| | (3Z)-3-[(3-methyl-1H-pyrazol-5-yl) | | |
| | methylene]indolin-2-one | | |
| 2 | 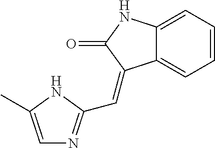 [SMILES: Cc1cnc(/C=C2\C(=O)Nc3ccccc32)[nH]1] | (DMSO-d6, 300 MHz) 1.28 (s, 3H), 6.97 (d, 1H), 7.09 (t, 1H), 7.17 (s, 1H), 7.30 (t, 1H), 7.78 (s, 1H), 7.86 (t, 1H), 11.2 (s, 1H), 14.0 (d, 1H) | 226 |
| | (3Z)-3-[(5-methyl-1H-imidazol-2- | | |
| | yl)methylene]indolin-2-one | | |
| 3 | 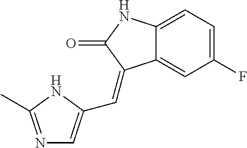 [SMILES: Cc1ncc(/C=C2\C(=O)Nc3ccc(F)cc32)[nH]1] | (DMSO-d6, 300 MHz) 2.44 (s, 3H), 6.84 (m, 1H), 6.98 (m, 1H), 7.53 (m, 2H), 7.81 (s, 1H), 10.9 (s, 1H), 13.5 (s, 1H) | 244 |
| | (3Z)-5-fluoro-3-[(2-methyl-1H-imidazol- | | |
| | 5-yl)methylene]indolin-2-one | | |
| 4 | 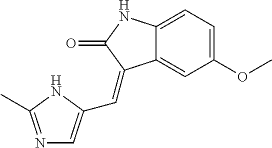 | (DMSO-d6, 300 MHz) 2.45 (s, 3H), 3.76 (s, 3H), 6.77 (m, 2H), 7.33 (s, 1H), 7.51 (s, 1H), 7.80 (s, 1H), 10.8 (s, 1H), 13.6 (s, 1H) | 256 |
| | (3Z)-5-methoxy-3-[(2-methyl-1H- | | |
| | imidazol-5-yl)methylene]indolin-2-one | | |
| 5 | 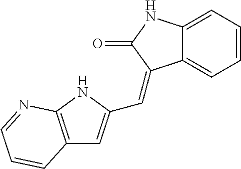 [SMILES: O=C1Nc2ccccc2/C1=C/c1cc2cccnc2[nH]1] | (DMSO-d6, 300 MHz) 6.93 (d, 1H), 7.04-7.29 (m, 4H), 7.76 (d, 1H), 7.98 (s, 1H), 8.12 (d, 1H), 8.40 (s, 1H), 11.2 (s, 1H), 13.3 (s, 1H) | 262 |
| | (3Z)-3-(1H-pyrrolo[2,3-b]pyridin-2- | | |
| | ylmethylene)indolin-2-one | | |
| 6 | 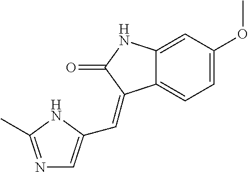 | (DMSO-d6, 300 MHz) 2.44 (s, 3H), 3.76 (s, 3H), 6.45 (s, 1H), 6.60 (d, 1H), 7.45 (s, 1H), 7.55 (m, 2H), 10.9 (s, 1H), 13.3 (s, 1H) | 256 |
| | (3Z)-6-methoxy-3-[(2-methyl-1H-imidazol- | | |
| | 5-yl)methylene]indolin-2-one | | |
| 7 | 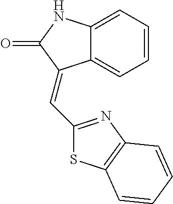 [SMILES: O=C1Nc2ccccc2/C1=C\c1nc2ccccc2s1] | (DMSO-d6, 300 MHz) 6.93 (d, 1H), 7.10 (t, 1H), 7.40 (t, 1H), 7.55- 7.68 (m, 3H), 8.27 (m, 2H), 9.25 (d, 1H), 10.8 (s, 1H) | 279 |
| | (3E)-3-(1,3-benzothiazol-2- | | |
| | ylmethylene)indolin-2-one | | |
| 8 | 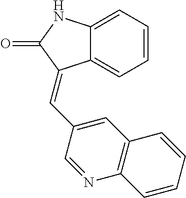 [SMILES: O=C1Nc2ccccc2/C1=C\c1cnc2ccccc2c1] | (DMSO-d6, 300 MHz) 6.87 (d, 1H), 7.04 (t, 1H), 7.26 (t, 1H), 7.64- 7.86 (m, 3H), 8.04 (m, 3H), 9.39 (s, 1H), 9.52 (s, 1H), 10.7 (s, 1H) | 273 |
| | (3E)-3-(3-quinolylmethylene) | | |
| | indolin-2-one | | |
| 9 | 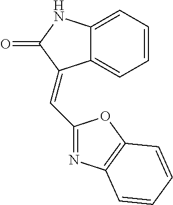 [SMILES: O=C1Nc2ccccc2/C1=C\c1nc2ccccc2o1] | (DMSO-d6, 300 MHz) 6.93 (d, 1H), 7.13 (t, 1H), 7.29 (s, 1H), 7.38- 7.58 (m, 3H), 7.87 (d, 1H), 7.97 (d, 1H), 9.08 (d, 1H), 10.8 (br s, 1H) | 263 |
| | (3E)-3-(1,3-benzoxazol-2- | | |
| | ylmethylene)indolin-2-one | | |
| 10 | 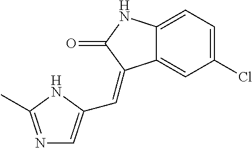 [SMILES: Cc1ncc(/C=C2\C(=O)Nc3ccc(Cl)cc32)[nH]1] | (300 MHz, DMSO-d₆) 13.43 (br s, 1H), 11.09 (br s, 1H), 7.90-7.77 (m, 2H), 7.55 (s, 1H), 7.21 (d, J = 8.1 Hz, 1H), 6.88 (d, J = 8.1 Hz, 1H), 2.49 (s, 3H) | 260 |
| | (3Z)-5-chloro-3-[(2-methyl-1H-imidazol- | | |
| | 5-yl)methylene]indolin-2-one | | |
| 11 | 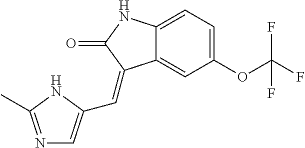 [SMILES: Cc1ncc(/C=C2\C(=O)Nc3ccc(OC(F)(F)F)cc32)[nH]1] | (300 MHz, DMSO-d₆) 13.44 (br s, 1H), 11.15 (br s, 1H), 7.97 (s, 1H), 7.83 (s, 1H), 7.57 (s, 1H), 7.17-7.11(m, 1H), 6.97-6.85 (m, 1H), 2.47 (s, 3H) | 310 |
| | (3Z)-3-[(2-methyl-1H-imidazol-5-yl) | | |
| | methylene]-5-(trifluoromethoxy)indolin-2-one | | |
| 12 | 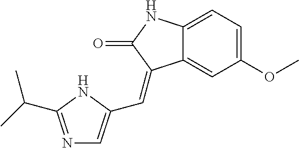 [SMILES: COc1ccc2c(c1)/C(=C/c1cnc(C(C)C)[nH]1)C(=O)N2] | (300 MHz, CDCl₃) 13.88 (br s, 1H), 7.75 (s, 1H), 7.58 (s, 1H), 7.40 (s, 1H), 7.07 (d, J = 2.1 Hz, 1H), 6.85-6.77 (m, 2H), 3.81 (s, 3H), 3.27- 3.20 (m, 1H), 1.45 (s, 6H) | 284 |
| | (3Z)-3-[(2-isopropyl-1H-imidazol-5-yl) | | |
| | methylene]-5-methoxy-indolin-2-one | | |
| 13 | 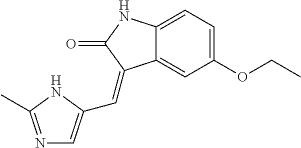 [SMILES: CCOc1ccc2c(c1)/C(=C/c1cnc(C)[nH]1)C(=O)N2] | (300 MHz, DMSO- d₆) 13.52 (br s, 1H), 10.78 (br s, 1H), 7.78 (s, 1H), 7.64 (d, J = 7.5 Hz, 1H), 7.32 (m, 1H), 6.85 (m, 2H), 3.99 (q, 2H), 2.47 (s, 3H), 1.34 (t, 3H) | 270 |
| | (3Z)-5-ethoxy-3-[(2-methyl-1H-imidazol- | | |
| | 5-yl)methylene]indolin-2-one | | |
| 14 | 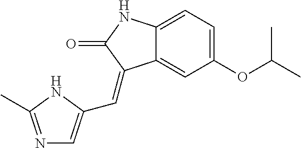 [SMILES: Cc1ncc(/C=C2\C(=O)Nc3ccc(OC(C)C)cc32)[nH]1] | (300 MHz, DMSO- d₆) 13.50 (br s, 1H), 10.30 (br s, 1H), 7.65 (s, 1H), 7.30 (s, 1H), 6.87 (s, 1H), 6.72 (m, 2H), 4.59-4.49 (m, 1H), 2.33 (s, 3H), 1.30 (s, 6H) | 284 |
| | (3Z)-5-isopropoxy-3-[(2-methyl-1H- | | |
| | imidazol-5-yl)methylene]indolin-2-one | | |
| 15 | 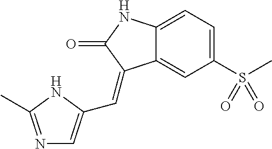 | (300 MHz, DMSO-d₆) 13.30 (br s, 1H), 11.46 (br s, 1H), 8.23 (d, J = 1.5 Hz, 1H), 8.08 (s, 1H), 7.91 (s, 1H), 7.73 (m, 1H), 7.65 (s, 1H), 7.53 (s, 1H), 7.10 (d, J = 8.1 Hz, 1H), 7.01 (d, J = 8.1 Hz, 1H), 3.35 (s, 3H), 2.50 (s, 3H) | 304 |
| | (3Z)-3-[(2-methyl-1H-imidazol-5-yl) | | |
| | methylene]-5-methylsulfonyl-indolin-2-one | | |
| 16 | 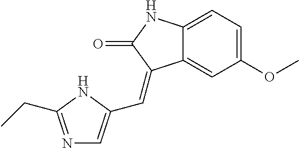 [SMILES: CCc1ncc(/C=C2\C(=O)Nc3ccc(OC)cc32)[nH]1] | (300 MHz, CDCl₃) 13.67 (br s, 1H), 7.94 (s, 1H), 7.56 (s, 1H), 7.39 (s, 1H), 7.06 (m, 1H), 6.84-6.77 (m, 2H), 3.86 (s, 3H), 2.95 (q, J = 7.5 Hz, 2H), 1.27 (t, J = 7.2 Hz, 3H) | 270 |
| | (3Z)-3-[(2-ethyl-1H-imidazol-5-yl) | | |
| | methylene]-5-methoxy-indolin-2-one | | |
| 17 | 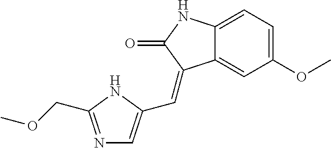 | (300 MHz, CDCl₃) 13.89 (br s, 1H), 7.72 (s, 1H), 7.58 (s, 1H), 7.40 (s, 1H), 7.07 (m, 1H), 6.85-6.77 (m, 2H), 4.45 (s, 2H), 3.96 (s, 3H), 3.81 (s, 3H) | 286 |
| | (3Z)-5-methoxy-3-[[2-(methoxymethyl)-1H- | | |
| | imidazol-5-yl]methylene]indolin-2-one | | |
| 18 | 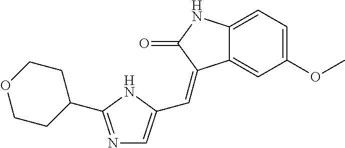 [SMILES: COc1ccc2c(c1)/C(=C/c1cnc(C3CCOCC3)[nH]1)C(=O)N2] | (300 MHz, DMSO-d₆) 10.82 (br s, 1H), 7.84 (s, 1H), 7.55 (s, 1H), 7.35 (s, 1H), 6.82-6.75 (m, 2H), 3.95 (m, 2H), 3.76 (s, 3H), 3.48 (m, 2H), 3.07 (m, 1H), 2.00 (m, 2H), 1.78 (m, 2H) | 326 |
| | (3Z)-5-methoxy-3-[(2-tetrahydropyran-4-yl- | | |
| | 1H-imidazol-5-yl)methylene]indolin-2-one | | |
| 19 | 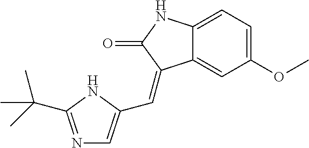 [SMILES: COc1ccc2c(c1)/C(=C/c1cnc(C(C)(C)C)[nH]1)C(=O)N2] | (300 MHz, DMSO-d₆) 14.08 (br s, 1H), 10.80 (br s, 1H), 7.85 (s, 1H), 7.53 (s, 1H), 7.34 (d, J = 2.1 Hz, 1H), 6.78 (m, 2H), 3.76 (s, 3H), 1.26 (s, 9H) | 298 |
| | (3Z)-3-[(2-tert-butyl-1H-imidazol-5-yl) | | |
| | methylene]-5-methoxy-indolin-2-one | | |
| 20 | 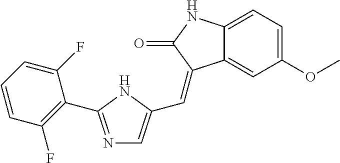 [SMILES: COc1ccc2c(c1)/C(=C/c1cnc(-c3c(F)cccc3F)[nH]1)C(=O)N2] | (300 MHz, DMSO-d₆) 14.67 (br s, 1H), 10.87 (br s, 1 H), 7.97 (s, 1H), 7.81 (m, 1H), 7.59 (m, 1H), 7.53 (m, 3H), 6.83 (m, 2H), 3.77 (s, 1H) | 354 |
| | (3Z)-3-[[2-(2,6-difluorophenyl)-1H-imidazol-5- | | |
| | yl]methylene]-5-methoxy-indolin-2-one | | |
| 21 | 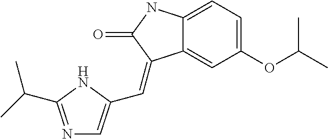 [SMILES: CC(C)Oc1ccc2c(c1)/C(=C/c1cnc(C(C)C)[nH]1)C(=O)N2] | (300 MHz, CDCl₃) 13.81 (br s, 1H), 8.37 (s, 1H), 7.56 (s, 1H), 7.37 (s, 1H), 7.07 (s, 1H), 6.80 (m, 2H), 4.51 (m, 1H), 3.23 (m, 1H), 1.45 (s, 12H) | 312 |
| | (3Z)-5-isopropoxy-3-[(2-isopropyl-1H- | | |
| | imidazol-5-yl)methylene]indolin-2-one | | |
| 22 | 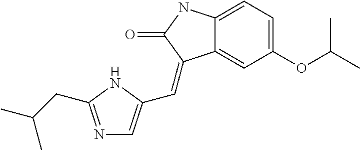 [SMILES: CC(C)Cc1ncc(/C=C2\C(=O)Nc3ccc(OC(C)C)cc32)[nH]1] | (300 MHz, CDCl₃) 13.75 (br s, 1H), 10.74 (s, 1H), 7.80 (s, 1H), 7.64 (s, 1H), 7.33 (s, 1H), 6.79-6.71 (m, 2H), 4.58-4.50 (m, 1H), 2.64 (m, 2H), 2.11-1.99 (m, 1H), 1.27 (s, 6H), 1.08 (s, 6H) | 326 |
| | (3Z)-3-[(2-isobutyl-1H-imidazol-5-yl) | | |
| | methylene]-5-isopropoxy-indolin-2-one | | |
| 23 | 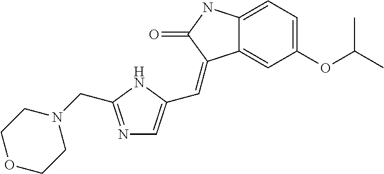 [SMILES: CC(C)Oc1ccc2c(c1)/C(=C/c1cnc(CN3CCOCC3)[nH]1)C(=O)N2] | (300 MHz, DMSO-d₆) 13.87 (br s, 1H), 10.82 (br s, 1H), 7.83 (s, 1H), 7.53 (s, 1H), 7.34 (s, 1H), 6.79-6.72 (m, 2H), 4.57-4.53 (m, 2H), 3.36 (m, 4H), 1.27 (m, 4H), 1.25 (s, 6H) | 369 |
| | (3Z)-5-isopropoxy-3-[[2-(morpholinomethyl)-1H- | | |
| | imidazol-5-yl]methylene]indolin-2-one | | |
| 24 | 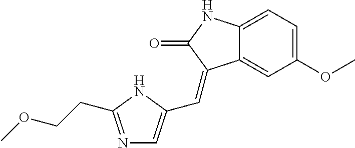 | (300 MHz, CDCl₃) 13.70 (br s, 1H), 8.10 (s, 1H), 7.55 (s, 1H), 7.35 (s, 1H), 7.05 (d, J = 2.1 Hz, 1H), 6.93-6.77 (m, 2H), 3.85 (s, 3H), 3.84 (t, J = 6.0 Hz, 2H), 3.49 (s, 3H), 3.19 (t, J = 6.0 Hz, 3H) | 300 |
| | (3Z)-5-methoxy-3-[[2-(2-methoxyethyl)-1H- | | |
| | imidazol-5-yl]methylene]indolin-2-one | | |
| 25 | 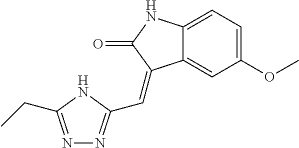 [SMILES: CCc1nnc(/C=C2\C(=O)Nc3ccc(OC)cc32)[nH]1] | (300 MHz, DMSO-d₆): δ 14.78 (br s, 1H), 11.11 (br s, 1H), 8.00 (s, 1H), 7.65 (s, 1H), 6.92- 6.83 (m, 2H), 3.76 (s, 3H), 2.74 (q, J = 7.2 Hz, 2H), 1.28 (t, J = 8.4 Hz, 3H) | 271 |
| | (3Z)-3-[(5-ethyl-4H-1,2,4-triazol-3-yl) | | |
| | methylene]-5-methoxy-indolin-2-one | | |
| 26 | 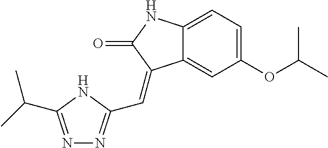 [SMILES: CC(C)Oc1ccc2c(c1)/C(=C/c1nnc(C(C)C)[nH]1)C(=O)N2] | (300 MHz, DMSO-d₆) 14.76 (br s, 1H), 11.10 (br s, 1H), 7.97 (s, 1H), 7.62 (d, J = 2.1 Hz, 1H), 6.86-6.80 (m, 2H), 4.57- 4.53 (m, 1H), 3.07 (m, 1H), 1.36 (s, 6H), 1.28 (s, 6H) | 313 |
| | (3Z)-5-isopropoxy-3-[(5-isopropyl-4H-1,2,4- | | |
| | triazol-3-yl)methylene]indolin-2-one | | |
| 27 | 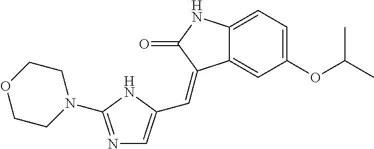 [SMILES: CC(C)Oc1ccc2c(c1)/C(=C/c1cnc(N3CCOCC3)[nH]1)C(=O)N2] | (300 MHz, DMSO-d₆): δ 13.69 (br s, 1H), 10.63 (br s, 1H), 7.62 (s, 1H), 7.43 (s, 1H), 7.23 (s, 1H), 6.74 (d, J = 8.4 Hz, 1H), 6.66 (d, J = 8.4 Hz, 1H), 4.52 (m, 1H), 3.72 (m, 4H), 3.42 (m, 4H), 1.24 (s, 6H) | 355 |
| | (3Z)-5-isopropoxy-3-[(2-morpholino-1H- | | |
| | imidazol-5-yl)methylene]indolin-2-one | | |
| 28 | 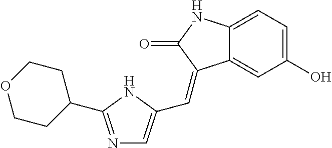 [SMILES: O=C1Nc2ccc(O)cc2/C1=C/c1cnc(C2CCOCC2)[nH]1] | (300 MHz, DMSO-d₆) 13.99 (br s, 1H), 10.71 (br s, 1H), 9.08 (s, 1H), 7.68 (s, 1H), 7.55 (s, 1H), 7.03 (s, 1H), 6.70 (m, 1H), 6.61 (m, 1H), 3.92 (m, 2H), 3.47 (m, 2H), 3.10 (m, 1H), 1.92 (m, 2H), 1.78- 1.69 (m, 2H) | 312 |
| | (3Z)-5-hydroxy-3-[(2-tetrahydropyran-4-yl-1H- | | |
| | imidazol-5-yl)methylene]indolin-2-one | | |
| 29 | 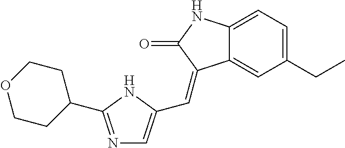 [SMILES: CCc1ccc2c(c1)/C(=C/c1cnc(C3CCOCC3)[nH]1)C(=O)N2] | (300 MHz, DMSO-d₆) 13.89 (br s, 1H), 10.90 (br s, 1H), 7.80 (s, 1H), 7.83 (s, 1H), 7.53 (d, J = 7.8 Hz, 1H), 7.02 (m, 1H), 6.82 (d, J = 7.8 Hz, 1H), 3.92 (m, 2H), 3.49 (m, 2H), 3.12 (m, 1H), 2.62 (m, 2H), 1.97 (m, 2H), 1.93 (m, 2H), 1.78 (m, 3H) | 324 |
| | (3Z)-5-ethyl-3-[(2-tetrahydropyran-4-yl-1H- | | |
| | imidazol-5-yl)methylene]indolin-2-one | | |
| 30 | 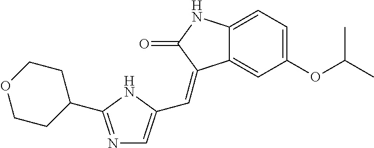 [SMILES: CC(C)Oc1ccc2c(c1)/C(=C/c1cnc(C3CCOCC3)[nH]1)C(=O)N2] | (300 MHz, DMSO-d₆) 13.96 (br s, 1H), 10.80 (br s, 1H), 7.84 (s, 1H), 7.53 (s, 1H), 7.34 (s, 1H), 6.78 (m, 2H), 4.55 (m, 1H), 3.92 (m, 2H), 3.47 (m, 2H), 3.10 (m, 1H), 1.95 (m, 2H), 1.73 (m, 2H), 1.25 (s, 6H) | 354 |
| | (3Z)-5-isopropoxy-3-[(2-tetrahydropyran-4-yl- | | |
| | 1H-imidazol-5-yl)methylene]indolin-2-one | | |
| 31 | 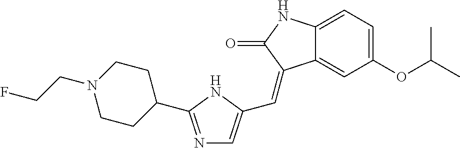 [SMILES: CC(C)Oc1ccc2c(c1)/C(=C/c1cnc(C3CCN(CCF)CC3)[nH]1)C(=O)N2] | (300 MHz, CDCl₃) 13.79 (br s, 1H), 7.82 (s, 1H), 7.55 (s, 1H), 7.37 (m, 1H), 6.80 (m, 2H), 4.71 (m, 1H), 4.52 (m, 2H), 3.10 (m, 2H), 2.88 (m, 1H), 2.82 (m, 1H), 2.71 (m, 1H), 2.33 (m, 1H), 2.27 (m, 2H), 2.06 (s, 6H) | 399 |
| | (3Z)-3-[[2-[1-(2-fluoroethyl)-4-piperidyl]-1H-imidazol- | | |
| | 5-yl]methylene]-5-isopropoxy-indolin-2-one | | |
| 32 | 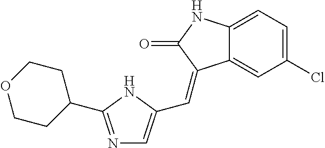 [SMILES: O=C1Nc2ccc(Cl)cc2/C1=C/c1cnc(C2CCOCC2)[nH]1] | (300 MHz, DMSO-d₆) 13.82 (br s, 1H), 11.12 (br s, 1H), 7.96 (s, 1H), 7.79 (s, 1H), 7.20 (s, 1H), 6.90 (m, 1H), 3.92 (m, 2H), 3.47 (m, 2H), 3.12 (m, 1H), 1.92 (m, 2H), 1.80- 1.67 (m, 2H) | 330 |
| | (3Z)-5-chloro-3-[(2-tetrahydropyran-4-yl- | | |
| | 1H-imidazol-5-yl)methylene]indolin-2-one | | |
| 33 | 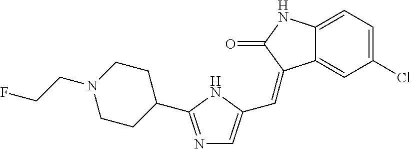 [SMILES: O=C1Nc2ccc(Cl)cc2/C1=C/c1cnc(C2CCN(CCF)CC2)[nH]1] | (300 MHz, CDCl₃) 13.74 (br s, 1H), 8.49 (s, 1H), 7.59 (s, 1H), 7.47 (s, 1H), 7.41 (s, 1H), 7.19 (s, 1H), 6.86 (m, 1H), 4.71 (m, 1H), 4.56 (m, 1H), 3.14 (m, 2H), 2.95 (m, 1H), 2.91 (m, 1H), 2.86 (m, 1H), 2.75 (m, 2H), 2.38 (m, 2H), 2.27 (m, 2H) | 375 |
| | (3Z)-5-chloro-3-[[2-[1-(2-fluoroethyl)-4-piperidyl]- | | |
| | 1H-imidazol-5-yl]methylene]indolin-2-one | | |
| 34 | 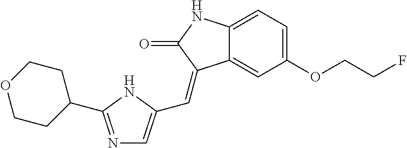 [SMILES: O=C1Nc2ccc(OCCF)cc2/C1=C/c1cnc(C2CCOCC2)[nH]1] | (300 MHz, DMSO-d₆) 13.96 (br s, 1H), 10.81 (br s, 1H), 7.83 (s, 1H), 7.55 (s, 1H), 7.40 (s, 1H), 6.80 (m, 2H), 4.70 (m, 2H), 4.28 (m, 2H), 3.93 (m, 2H), 3.51 (m, 2H), 3.01 (m, 1H), 1.94 (m, 2H), 1.79 (m, 2H) | 358 |
| | (3Z)-5-(2-fluoroethoxy)-3-[(2-tetrahydropyran-4-yl- | | |
| | 1H-imidazol-5-yl)methylene]indolin-2-one | | |
| 35 | 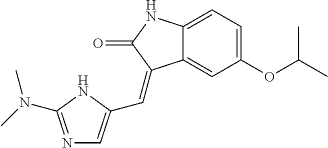 [SMILES: CC(C)Oc1ccc2c(c1)/C(=C/c1cnc(N(C)C)[nH]1)C(=O)N2] | (300 MHz, DMSO-d₆) 13.54 (br s, 1H), 10.57 (br s, 1H), 7.54 (s, 1H), 7.41 (s, 1H), 7.19 (d, J = 1.8 Hz, 1H), 6.73 (d, J = 8.4 Hz, 1H), 6.63 (m, 1H), 4.51 (m, 1H), 3.07 (s, 6H), 1.23 (s, 6H) | 313 |
| | (3Z)-3-[[2-(dimethylamino)-1H-imidazol-5- | | |
| | yl]methylene]-5-isopropoxy-indolin-2-one | | |
| 36 | 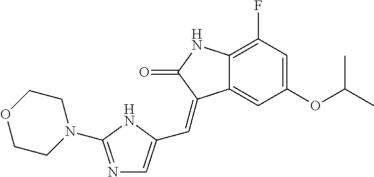 [SMILES: CC(C)Oc1cc(F)c2c(c1)/C(=C/c1cnc(N3CCOCC3)[nH]1)C(=O)N2] | (DMSO-d6, 300 MHz) 1.25 (s, 6H), 3.43 (m, 4H), 3.72 (m, 4H), 4.56 (m, 1H), 6.62 (dd, 1H), 7.10 (s, 1H), 7.50 (s, 1H), 7.69 (s, 1H), 11.1 (br s, 1H), 13.6 (br s, 1H) | 373 |
| | (3Z)-7-fluoro-5-isopropoxy-3-[(2-morpholino-1H- | | |
| | imidazol-5-yl)methylene]indolin-2-one | | |
| 37 | 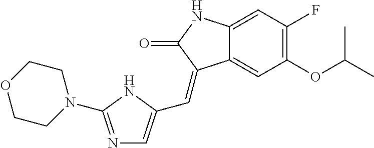 [SMILES: CC(C)Oc1cc2c(cc1F)NC(=O)/C2=C\c1cnc(N2CCOCC2)[nH]1] | (DMSO-d6, 300 MHz) 1.28 (s, 6H), 3.42 (m, 4H), 3.72 (m, 4H), 4.52 (m, 1H), 6.66 (d, 1H), 6.72 (d, 1H), 7.43 (s, 1H), 7.63 (s, 1H), 10.7 (br s, 1H), 13.5 (br s, 1H) | 373 |
| | (3Z)-6-fluoro-5-isopropoxy-3-[(2-morpholino-1H- | | |
| | imidazol-5-yl)methylene]indolin-2-one | | |
| 38 | 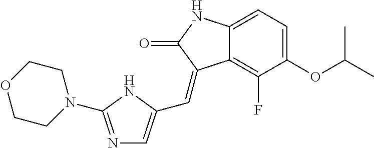 [SMILES: CC(C)Oc1ccc2c(c1F)/C(=C/c1cnc(N3CCOCC3)[nH]1)C(=O)N2] | (DMSO-d6, 300 MHz) 1.25 (s, 6H), 3.43 (m, 4H), 3.71 (m, 4H), 4.37 (m, 1H), 6.62 (d, 1H), 6.89 (d, 1H), 7.51 (s, 1H), 7.64 (s, 1H), 10.9 (br s, 1H), 13.6 (br s, 1H) | 373 |
| | (3Z)-4-fluoro-5-isopropoxy-3-[(2-morpholino- | | |
| | 1H-imidazol-5-yl)methylene]indolin-2-one | | |
| 39 | 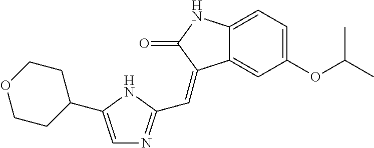 [SMILES: CC(C)Oc1ccc2c(c1)/C(=C/c1ncc(C3CCOCC3)[nH]1)C(=O)N2] | (DMSO-d6, 300 MHz) 1.26 (s, 6H), 1.71 (s, 2H), 1.87 (m, 2H), 3.96 (m, 2H), 4.56 (m, 2H), 4.60 (m, 2H), 6.78 (m, 2H), 7.33 (s, 1H) 7.50 (s, 1H), 7.80 (s, 1H), 10.7 (br s, 1H), 14.0 (br s, 1H) | 354 |
| | (3Z)-5-isopropoxy-3-[(5-tetrahydropyran-4- | | |
| | yl-1H-imidazol-2-yl)methylene]indolin-2-one | | |
| 40 | 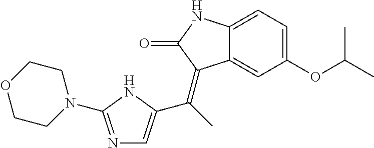 [SMILES: C/C(=C1/C(=O)Nc2ccc(OC(C)C)cc21)c1cnc(N2CCOCC2)[nH]1] | (DMSO-d6, 300 MHz) 1.26 (s, 6H), 2.60 (s, 3H), 3.40 (m, 4H), 3.74 (m, 4H), 4.51 (m, 1H), 6.80 (m, 2H), 7.17 (s, 1H), 7.75 (s, 1H), 10.7 (br s, 1H), 15.3 (br s, 1H) | 369 |
| | (3Z)-5-isopropoxy-3-[1-(2-morpholino-1H- | | |
| | imidazol-5-yl)ethylidene]indolin-2-one | | |
| 41 | 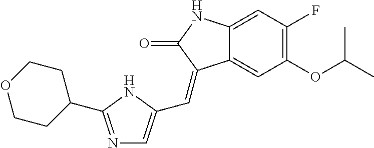 [SMILES: CC(C)Oc1cc2c(cc1F)NC(=O)/C2=C\c1cnc(C2CCOCC2)[nH]1] | (DMSO-d6, 300 MHz) 1.27 (s, 6H), 1.79 (m, 2H), 1.98 (m, 2H), 3.12 (m, 1H), 3.49 (m, 2H), 3.92 (m, 2H), 4.49-4.53 (m, 1H), 6.76 (d, 1H), 7.60 (m, 2H), 7.84 (s, 1H), 10.9 (br s, 1H), 13.8 (br s, 1H) | 372 |
| | (3Z)-6-fluoro-5-isopropoxy-3-[(2-tetrahydropyran- | | |
| | 4-yl-1H-imidazol-5-yl)methylene]indolin-2-one | | |
| 42 | 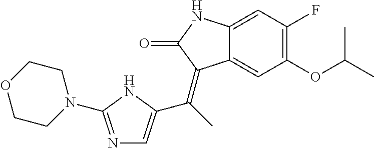 [SMILES: C/C(=C1/C(=O)Nc2cc(F)c(OC(C)C)cc21)c1cnc(N2CCOCC2)[nH]1] | (DMSO-d6, 300 MHz) 1.27 (s, 6H), 2.51 (s, 3H), 3.41 (m, 4H), 3.73 (m, 4H), 4.45 (m, 1H), 6.78 (d, 1H), 7.37 (d, 1H), 7.77 (s, 1H), 10.9 (br s, 1H), 15.1 (br s, 1H) | 387 |
| | (3Z)-6-fluoro-5-isopropoxy-3-[1-(2-morpholino- | | |
| | 1H-imidazol-5-yl)ethylidene]indolin-2-one | | |
| 43 | 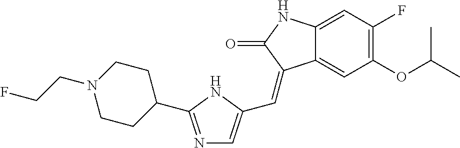 [SMILES: CC(C)Oc1cc2c(cc1F)NC(=O)/C2=C\c1cnc(C2CCN(CCF)CC2)[nH]1] | (DMSO-d6, 300 MHz) 1.27 (s, 6H), 1.75 (m, 2H), 2.02 (m, 2H), 2.21 (m, 2H), 2.71 (m, 2H), 2.85 (m, 1H) 2.97 (m, 2H) 4.62-4.65 (m, 1H), 6.78 (d, 1H), 7.60 (m, 2H), 7.83 (s, 1H), 10.9 (br s, 1H), 13.8 (br s, 1H) | 417 |
| | (3Z)-6-fluoro-3-[[2-[1-(2-fluoroethyl)-4-piperidyl]-1H- | | |
| | imidazol-5-yl]methylene]-5-isopropoxy-indolin-2-one | | |
| 44 | 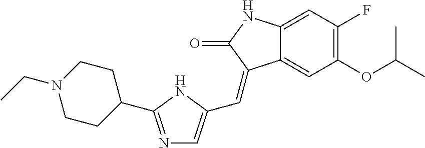 [SMILES: CCN1CCC(c2ncc(/C=C3\C(=O)Nc4cc(F)c(OC(C)C)cc43)[nH]2)CC1] | (DMSO-d6, 300 MHz) 1.26 (t, 3H), 1.27 (s, 6H), 2.32 (m, 2H), 2.51 (m, 4H), 3.32-3.45 (m, 4H), 4.48 (m, 1H), 6.72 (m, 1H), 7.44 (m, 2H), 7.61 (s, 1H), 10.7 (br s, 1H), 13.5 (br s, 1H) | 400 |
| | (3Z)-3-[[2-(4-ethylpiperazin-1-yl)-1H-imidazol-5-yl] | | |
| | methylene]-6-fluoro-5-isopropoxy-indolin-2-one | | |
| 45 | 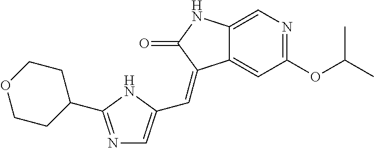 [SMILES: CC(C)Oc1cc2c(cn1)NC(=O)/C2=C\c1cnc(C2CCOCC2)[nH]1] | (DMSO-d6, 300 MHz) 1.26 (s, 6H), 1.72 (s, 2H), 1.96 (m, 2H), 3.09 (m, 1H), 3.34 (m, 2H), 3.96 (m, 2H) 5.19 (s, 1H), 7.09 (s, 1H), 7.77 (m, 1H), 8.01 (s, 1H), 11.0 (br s, 1H), 14.0 (br s, 1H) | 355 |
| | (3Z)-5-isopropoxy-3-[(2-tetrahydropyran-4-yl- | | |
| | 1H-imidazol-5-yl)methylene]-1H-pyrrolo[2,3-c] | | |
| | pyridin-2-one | | |
| 46 | 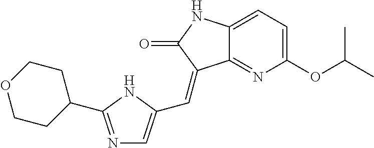 [SMILES: CC(C)Oc1ccc2c(n1)/C(=C/c1cnc(C3CCOCC3)[nH]1)C(=O)N2] | (DMSO-d6, 300 MHz) 1.26 (s, 6H), 1.81-1.97 (m, 4H), 3.51 (m, 2H), 3.96 (m, 2H), 4.97 (m, 1H), 6.78 (m, 2H), 7.30 (s, 1H), 7.55 (s, 1H), 7.88 (s, 1H), 10.7 (br s, 1H), 14.0 (br s, 1H) | 355 |
| | (3Z)-5-isopropoxy-3-[(2-tetrahydropyran-4- | | |
| | yl-1H-imidazol-5-yl)methylene]-1H-pyrrolo | | |
| | [3,2-b]pyridin-2-one | | |

### Example 3

### In vitro Assay

Compounds of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) were tested using DiscoveRx's KINOMEscan™ technology to measure a binding constant (Kd) to wild type and G2019S LRRK2. Kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). All reactions were performed in polystyrene 96-well plates in a final volume of 0.135 ml. The assay plates were incubated at room temperature with shaking for 1 hour and the affinity beads were washed with wash buffer (lx PBS, 0.05% Tween 20). The beads were then re-suspended in elution buffer (lx PBS, 0.05% Tween 20, 0.5 μM non-biotinylated affinity ligand) and incubated at room temperature with shaking for 30 minutes. The kinase concentration in the eluates was measured by qPCR. An 11-point 3-fold serial dilution of each test compound was prepared in 100% DMSO at 100× final test concentration and subsequently diluted to 1× in the assay (final DMSO concentration=2.5%). Most Kds were determined using a compound top concentration=30,000 nM.

IC50s were determined using Invitrogen's SelectScreen® Profiling Service for wild type and LRRK2 G2019S. The test compounds were screened in 1% DMSO (final) in the well.

a) % inhibition is measured at one concentration

b) a 10-point titration is run to ensure the kinase is inhibited within an expected IC₅₀ range

Biochemical Assay Protocol:

Compound potency against wild-type and G2019S LRRK2 is measured via TR-FRET. The IC50, the amount of inhibitor required to inhibitor 50% of the protein under the given assay conditions, is determined by varying the amount of compound (500-0.001 uM) by serial dilution with 12-data points per compound, in duplicate. The substrate (Fluorescein-GAGRLGRDKYKTLRQIRQ) and ATP is fixed at 0.8 uM and 57 uM (KmATP), respectively. All assays are run in Corning low volume 394-well round bottomed plates. The assay is run in 50 mM Tris-HCl pH8.5, 10 mM MgCl2, 0.01% Brij-35, 1 mM EGTA, 2 mMDTT. The TR-FRET assay is an end-point assay where the reaction with protein, substrate, inhibitor and ATP is incubated for 1 hour at room temperature. The compound is pre-incubated with the protein and substrate prior to initiating the reaction with ATP. Phospho-transfer is detected using a Terbium labeled anti-pLRRKtide that binds to phosphorylated substrate creating FRET transfer when excited at 340 nm. Emissions signals are taken for terbium and FRET induced fluorescein at 495 nm and 520 nm, respectively, using a Biotek Synergy 2 spectrophotometer. The dose response is determined by plotting the emission ratio of 520/495 against the log of the inhibitor concentration. The maximum inhibition and non-inhibition controls is run on all plates. Staurosporine is used as the positive inhibition control and DMSO is used as the negative point control. A 12-point dose curve of Staurosporine is run as a dosing control. The IC50 is determined to be the inhibitor concentration at which 50% inhibition is achieved. The protein construct used in the assay is GST tagged 969-2527. The protein is commercially available from Invitrogen.

Table 2 shows in vitro assay data for certain compounds of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d).

TABLE 2

| | | | G2019S | |
|---|---|---|---|---|
| | | G2019S | LRRK2 | G2019S |
| | | LRRK2 | IC₅₀ | LRRK2 |
| Compound | Structure | % inhibition | (μM) | K_{d} (μM) |
| 1 | 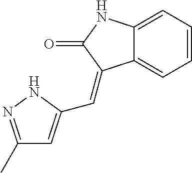 [SMILES: Cc1cc(/C=C2\C(=O)Nc3ccccc32)[nH]n1] | 22% @ 0.1 μM | | |
| | (3Z)-3-[(3-methyl-1H-pyrazol-5-yl) | | | |
| | methylene]indolin-2-one | | | |
| 2 | 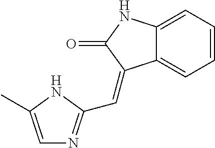 [SMILES: Cc1cnc(/C=C2\C(=O)Nc3ccccc32)[nH]1] | | B 0.181 | |
| | (3Z)-3-[(5-methyl-1H-imidazol-2- | | | |
| | yl)methylene]indolin-2-one | | | |
| 3 | 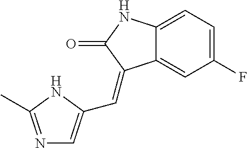 [SMILES: Cc1ncc(/C=C2\C(=O)Nc3ccc(F)cc32)[nH]1] | | A 0.038 | |
| | (3Z)-5-fluoro-3-[(2-methyl-1H-imidazol- | | | |
| | 5-yl)methylene]indolin-2-one | | | |
| 4 | 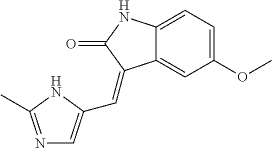 | | A 0.0069 | |
| | (3Z)-5-methoxy-3-[(2-methyl-1- | | | |
| | imidazol-5-yl)methylene]indolin-2-one | | | |
| 5 | 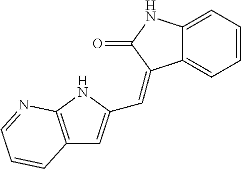 [SMILES: O=C1Nc2ccccc2/C1=C/c1cc2cccnc2[nH]1] | 30% @ 10 μM | | |
| | (3Z)-3-(1H-pyrrolo[2,3-b]pyridin-2- | | | |
| | ylmethylene)indolin-2-one | | | |
| 6 | 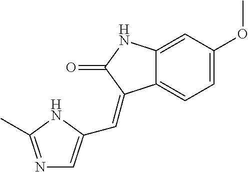 | | B 0.127 | |
| | (3Z)-6-methoxy-3-[(2-methyl-1H-imidazol- | | | |
| | 5-yl)methylene]indolin-2-one | | | |
| 7 | 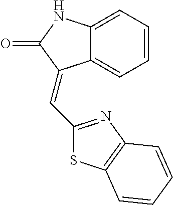 [SMILES: O=C1Nc2ccccc2/C1=C\c1nc2ccccc2s1] | −5% @ 0.1 μM | | |
| | (3E)-3-(1,3-benzothiazol-2- | | | |
| | ylmethylene)indolin-2-one | | | |
| 8 | 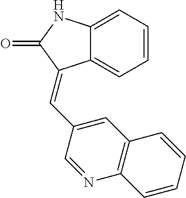 [SMILES: O=C1Nc2ccccc2/C1=C\c1cnc2ccccc2c1] | | C 0.872 | |
| | (3E)-3-(3-quinolylmethylene) | | | |
| | indolin-2-one | | | |
| 9 | 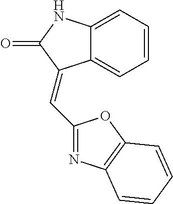 [SMILES: O=C1Nc2ccccc2/C1=C\c1nc2ccccc2o1] | 11% @ 10 μM | | |
| | (3E)-3-(1,3-benzoxazol-2- | | | |
| | ylmethylene)indolin-2-one | | | |
| 10 | 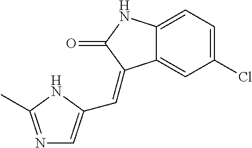 [SMILES: Cc1ncc(/C=C2\C(=O)Nc3ccc(Cl)cc32)[nH]1] | | A 0.0164 | |
| | (3Z)-5-chloro-3-[(2-methyl-1H-imidazol- | | | |
| | 5-yl)methylene]indolin-2-one | | | |
| 11 | 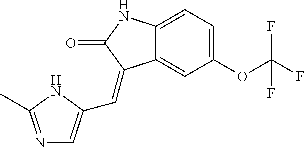 [SMILES: Cc1ncc(/C=C2\C(=O)Nc3ccc(OC(F)(F)F)cc32)[nH]1] | | A 0.0412 | |
| | (3Z)-3-[(2-methyl-1H-imidazol-5-yl) | | | |
| | methylene]-5-(trifluoromethoxy)indolin-2-one | | | |
| 12 | 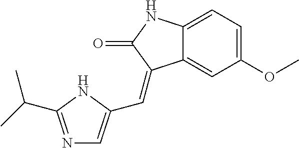 [SMILES: COc1ccc2c(c1)/C(=C/c1cnc(C(C)C)[nH]1)C(=O)N2] | | A 0.0076 | |
| | (3Z)-3-[(2-isopropyl-1H-imidazol-5-yl) | | | |
| | methylene]-5-methoxy-indolin-2-one | | | |
| 13 | 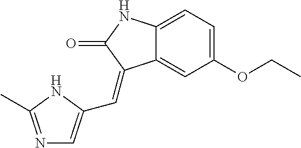 [SMILES: CCOc1ccc2c(c1)/C(=C/c1cnc(C)[nH]1)C(=O)N2] | | A 0.0031 | |
| | (3Z)-5-ethoxy-3-[(2-methyl-1H-imidazol- | | | |
| | 5-yl)methylene]indolin-2-one | | | |
| 14 | 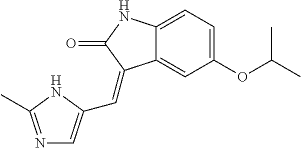 [SMILES: Cc1ncc(/C=C2\C(=O)Nc3ccc(OC(C)C)cc32)[nH]1] | | A 0.0012 | |
| | (3Z)-5-isopropoxy-3-[(2-methyl-1H- | | | |
| | imidazol-5-yl)methylene]indolin-2-one | | | |
| 15 | 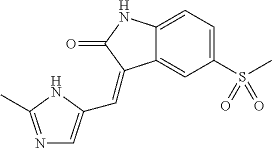 | | B 0.125 | |
| | (3Z)-3-[(2-methyl-1H-imidazol-5-yl) | | | |
| | methylene]-5-methylsulfonyl-indolin-2-one | | | |
| 16 | 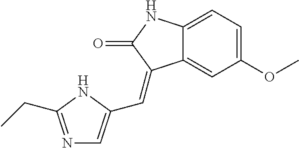 [SMILES: CCc1ncc(/C=C2\C(=O)Nc3ccc(OC)cc32)[nH]1] | | A 0.0035 | |
| | (3Z)-3-[(2-ethyl-1H-imidazol-5-yl) | | | |
| | methylene]-5-methoxy-indolin-2-one | | | |
| 17 | 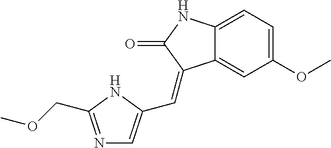 | | A 0.0387 | |
| | (3Z)-5-methoxy-3-[[2-(methoxymethyl)-1H- | | | |
| | imidazol-5-yl]methylene]indolin-2-one | | | |
| 18 | 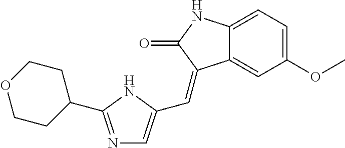 [SMILES: COc1ccc2c(c1)/C(=C/c1cnc(C3CCOCC3)[nH]1)C(=O)N2] | | A 0.0055 | A 0.095 |
| | (3Z)-5-methoxy-3-[(2-tetrahydropyran-4-yl- | | | |
| | 1H-imidazol-5-yl)methylene]indolin-2-one | | | |
| 19 | 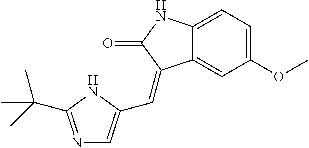 [SMILES: COc1ccc2c(c1)/C(=C/c1cnc(C(C)(C)C)[nH]1)C(=O)N2] | | A 0.0439 | |
| | (3Z)-3-[(2-tert-butyl-1H-imidazol-5-yl) | | | |
| | methylene]-5-methoxy-indolin-2-one | | | |
| 20 | 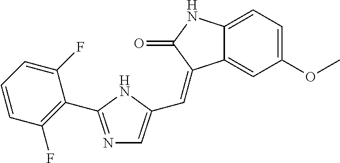 [SMILES: COc1ccc2c(c1)/C(=C/c1cnc(-c3c(F)cccc3F)[nH]1)C(=O)N2] | | | B 0.15 |
| | (3Z)-3-[[2-(2,6-difluorophenyl)-1H-imidazol-5- | | | |
| | yl]methylene]-5-methoxy-indolin-2-one | | | |
| 21 | 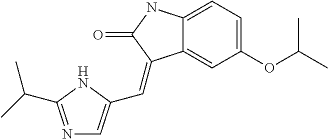 [SMILES: CC(C)Oc1ccc2c(c1)/C(=C/c1cnc(C(C)C)[nH]1)C(=O)N2] | | | A 0.0165 |
| | (3Z)-5-isopropoxy-3-[(2-isopropyl-1H- | | | |
| | imidazol-5-yl)methylene]indolin-2-one | | | |
| 22 | 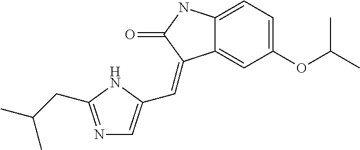 [SMILES: CC(C)Cc1ncc(/C=C2\C(=O)Nc3ccc(OC(C)C)cc32)[nH]1] | | | A 0.0325 |
| | (3Z)-3-[(2-isobutyl-1H-imidazol-5-yl) | | | |
| | methylene]-5-isopropoxy-indolin-2-one | | | |
| 23 | 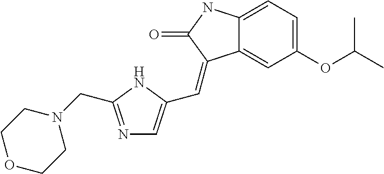 [SMILES: CC(C)Oc1ccc2c(c1)/C(=C/c1cnc(CN3CCOCC3)[nH]1)C(=O)N2] | | | B 0.42 |
| | (3Z)-5-isopropoxy-3-[[2-(morpholinomethyl)-1H- | | | |
| | imidazol-5-yl]methylene]indolin-2-one | | | |
| 24 | 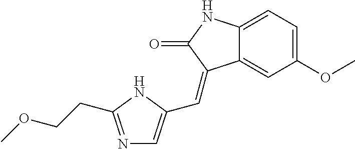 | | | B 0.45 |
| | (3Z)-5-methoxy-3-[[2-(2-methoxyethyl)-1H- | | | |
| | imidazol-5-yl]methylene]indolin-2-one | | | |
| 25 | 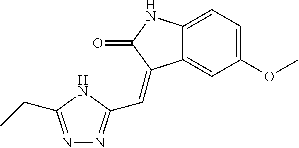 [SMILES: CCc1nnc(/C=C2\C(=O)Nc3ccc(OC)cc32)[nH]1] | | | D >30 |
| | (3Z)-3-[(5-ethyl-4H-1,2,4-triazol-3-yl) | | | |
| | methylene]-5-methoxy-indolin-2-one | | | |
| 26 | 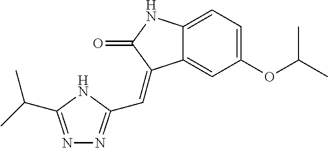 [SMILES: CC(C)Oc1ccc2c(c1)/C(=C/c1nnc(C(C)C)[nH]1)C(=O)N2] | | | D 12.5 |
| | (3Z)-5-isopropoxy-3-[(5-isopropyl-4H-1,2,4- | | | |
| | triazol-3-yl)methylene]indolin-2-one | | | |
| 27 | 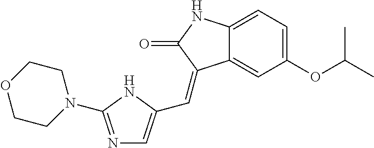 [SMILES: CC(C)Oc1ccc2c(c1)/C(=C/c1cnc(N3CCOCC3)[nH]1)C(=O)N2] | | | A 0.0155 |
| | (3Z)-5-isopropoxy-3-[(2-morpholino-1H- | | | |
| | imidazol-5-yl)methylene]indolin-2-one | | | |
| 28 | 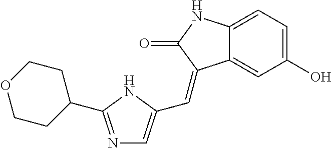 [SMILES: O=C1Nc2ccc(O)cc2/C1=C/c1cnc(C2CCOCC2)[nH]1] | | | A 0.068 |
| | (3Z)-5-hydroxy-3-[(2-tetrahydropyran-4-yl-1H- | | | |
| | imidazol-5-yl)methylene]indolin-2-one | | | |
| 29 | 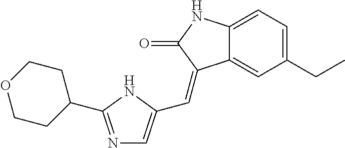 [SMILES: CCc1ccc2c(c1)/C(=C/c1cnc(C3CCOCC3)[nH]1)C(=O)N2] | | | A 0.054 |
| | (3Z)-5-ethyl-3-[(2-tetrahydropyran-4-yl-1H- | | | |
| | imidazol-5-yl)methylene]indolin-2-one | | | |
| 30 | 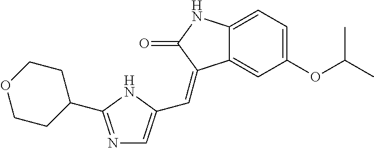 [SMILES: CC(C)Oc1ccc2c(c1)/C(=C/c1cnc(C3CCOCC3)[nH]1)C(=O)N2] | | | A 0.028 |
| | (3Z)-5-isopropoxy-3-[(2-tetrahydropyran-4-yl- | | | |
| | 1H-imidazol-5-yl)methylene]indolin-2-one | | | |
| 31 | 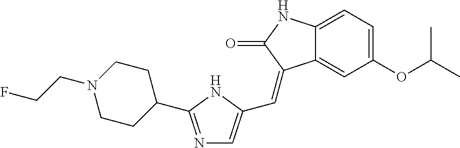 [SMILES: CC(C)Oc1ccc2c(c1)/C(=C/c1cnc(C3CCN(CCF)CC3)[nH]1)C(=O)N2] | | | A 0.043 |
| | (3Z)-3-[[2-[1-(2-fluoroethyl)-4-piperidyl]-1H-imidazol- | | | |
| | 5-yl]methylene]-5-isopropoxy-indolin-2-one | | | |
| 32 | 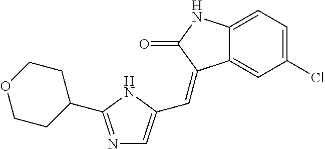 [SMILES: O=C1Nc2ccc(Cl)cc2/C1=C/c1cnc(C2CCOCC2)[nH]1] | | | A 0.059 |
| | (3Z)-5-chloro-3-[(2-tetrahydropyran-4-yl- | | | |
| | 1H-imidazol-5-yl)methylene]indolin-2-one | | | |
| 33 | 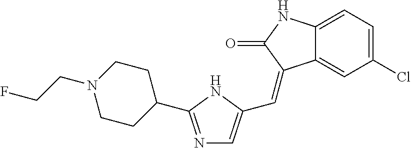 [SMILES: O=C1Nc2ccc(Cl)cc2/C1=C/c1cnc(C2CCN(CCF)CC2)[nH]1] | | | B 0.15 |
| | (3Z)-5-chloro-3-[[2-[1-(2-fluoroethyl)-4-piperidyl]- | | | |
| | 1H-imidazol-5-yl]methylene]indolin-2-one | | | |
| 34 | 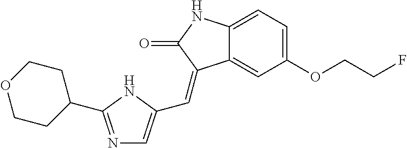 [SMILES: O=C1Nc2ccc(OCCF)cc2/C1=C/c1cnc(C2CCOCC2)[nH]1] | | | A 0.0475 |
| | (3Z)-5-(2-fluoroethoxy)-3-[(2-tetrahydropyran-4-yl- | | | |
| | 1H-imidazol-5-yl)methylene]indolin-2-one | | | |
| 35 | 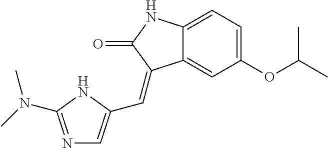 [SMILES: CC(C)Oc1ccc2c(c1)/C(=C/c1cnc(N(C)C)[nH]1)C(=O)N2] | | | A (0.032) |
| | (3Z)-3-[[2-(dimethylamino)-1H-imidazol-5- | | | |
| | yl]methylene]-5-isopropoxy-indolin-2-one | | | |
| 36 | 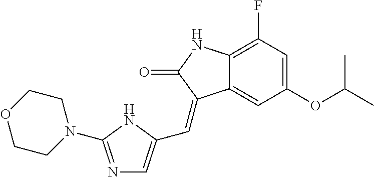 [SMILES: CC(C)Oc1cc(F)c2c(c1)/C(=C/c1cnc(N3CCOCC3)[nH]1)C(=O)N2] | | | A 0.067 |
| | (3Z)-7-fluoro-5-isopropoxy-3-[(2-morpholino-1H- | | | |
| | imidazol-5-yl)methylene]indolin-2-one | | | |
| 37 | 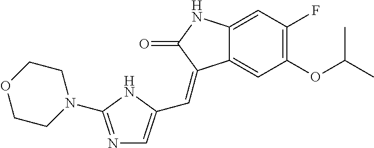 [SMILES: CC(C)Oc1cc2c(cc1F)NC(=O)/C2=C\c1cnc(N2CCOCC2)[nH]1] | | | A 0.024 |
| | (3Z)-6-fluoro-5-isopropoxy-3-[(2-morpholino-1H- | | | |
| | imidazol-5-yl)methylene]indolin-2-one | | | |
| 38 | 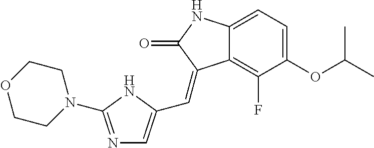 [SMILES: CC(C)Oc1ccc2c(c1F)/C(=C/c1cnc(N3CCOCC3)[nH]1)C(=O)N2] | | | D 2300 |
| | (3Z)-4-fluoro-5-isopropoxy-3-[(2-morpholino- | | | |
| | 1H-imidazol-5-yl)methylene]indolin-2-one | | | |
| 39 | 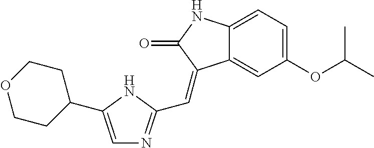 [SMILES: CC(C)Oc1ccc2c(c1)/C(=C/c1ncc(C3CCOCC3)[nH]1)C(=O)N2] | | | 0.123 |
| | (3Z)-5-isopropoxy-3-[(5-tetrahydropyran-4- | | | |
| | yl-1H-imidazol-2-yl)methylene]indolin-2-one | | | |
| 40 | 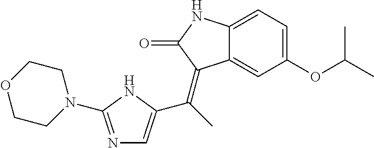 [SMILES: C/C(=C1/C(=O)Nc2ccc(OC(C)C)cc21)c1cnc(N2CCOCC2)[nH]1] | | | A 0.022 |
| | (3Z)-5-isopropoxy-3-[1-(2-morpholino-1H- | | | |
| | imidazol-5-yl)ethylidene]indolin-2-one | | | |
| 41 | 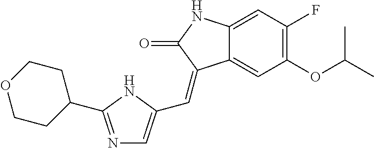 [SMILES: CC(C)Oc1cc2c(cc1F)NC(=O)/C2=C\c1cnc(C2CCOCC2)[nH]1] | | | A 0.025 |
| | (3Z)-6-fluoro-5-isopropoxy-3-[(2-tetrahydropyran- | | | |
| | 4-yl-1H-imidazol-5-yl)methylene]indolin-2-one | | | |
| 42 | 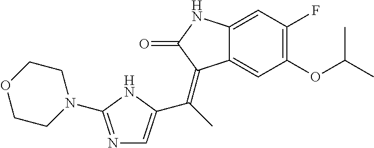 [SMILES: C/C(=C1/C(=O)Nc2cc(F)c(OC(C)C)cc21)c1cnc(N2CCOCC2)[nH]1] | | | A 0.057 |
| | (3Z)-6-fluoro-5-isopropoxy-3-[1-(2-morpholino- | | | |
| | 1H-imidazol-5-yl)ethylidene]indolin-2-one | | | |
| 43 | 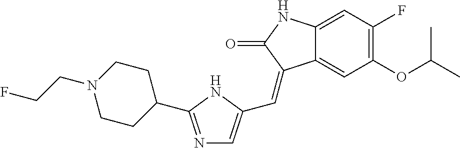 [SMILES: CC(C)Oc1cc2c(cc1F)NC(=O)/C2=C\c1cnc(C2CCN(CCF)CC2)[nH]1] | | | A 0.034 |
| | (3Z)-6-fluoro-3-[[2-[1-(2-fluoroethyl)-4-piperidyl]-1H- | | | |
| | imidazol-5-yl]methylenel-5-isopropoxy-indolin-2-one | | | |
| 44 | 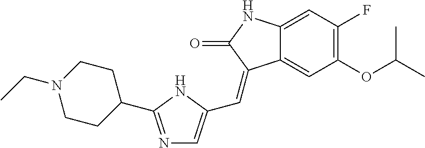 [SMILES: CCN1CCC(c2ncc(/C=C3\C(=O)Nc4cc(F)c(OC(C)C)cc43)[nH]2)CC1] | | | A 0.041 |
| | (3Z)-3-[[2-(4-ethylpiperazin-1-yl)-1H-imidazol-5-yl] | | | |
| | methylene]-6-fluoro-5-isopropoxy-indolin-2-one | | | |
| 45 | 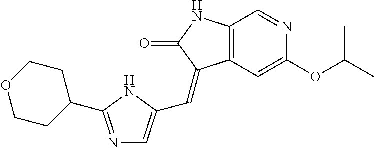 [SMILES: CC(C)Oc1cc2c(cn1)NC(=O)/C2=C\c1cnc(C2CCOCC2)[nH]1] | | | B 0.26 |
| | (Z)-5-isopropoxy-3-((2-(tetrahydro-2H-pyran-4-yl)- | | | |
| | 1H-imidazol-5-yl)methylene)- | | | |
| | 1H-pyrrolo[2,3-c]pyridin-2(3H)-one | | | |
| 46 | 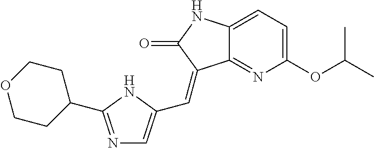 [SMILES: CC(C)Oc1ccc2c(n1)/C(=C/c1cnc(C3CCOCC3)[nH]1)C(=O)N2] | | | B 0.25 |
| | (3Z)-5-isopropoxy-3-[(2-tetrahydropyran-4- | | | |
| | yl-1H-imidazol-5-yl)methylene]-1H-pyrrolo | | | |
| | [3,2-b]pyridin-2-one | | | |

A refers to IC₅₀ or K_{d} of >0 and <100 nM,
B refers to IC₅₀ or K_{d} of ≧100 nM and <500 nM,
C refers to IC₅₀ or K_{d} of ≧500 nM and <1 microM,
D refers to IC₅₀ or K_{d} of ≧1 microM

Alternatively, compounds of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d) were tested using Invitrogen's SelectScreen® Profiling Service for LRRK2 G2019S.

Table 3 shows selectivity data against a broad panel of kinases for specific compounds of formulas (II), (I), (I-A), (I-B), (I-C), (I-D), (I-D-1), (I-D-2), (I-E), (I-F), (I-G), (I-H), (I-I), (I-I-1), (I-I-2), (I-J), (I-K), (I-L), (I-M), (I-N), (I-Z-1), (I-Z-2), (I-Z-2-a), (I-Z-2-b), (I-Z-2-c), (I-Z-2-d), (I-Z-3-a), (I-Z-3-b), (I-Z-3-c), or (I-Z-3-d).

TABLE 3

| | Cpd 18 | Cpd 18 | Cpd 12 | Cpd 14 | Cpd 19 |
|---|---|---|---|---|---|
| | % inhibition | IC₅₀ | IC₅₀ | IC₅₀ | IC₅₀ |
| Kinases | @0.1 uM | (uM) | (uM) | (uM) | (uM) |
| ABL1 | 5 | >10 | >10 | 0.86 | >10 |
| ALK4 | −3 | | | | |
| AKT1 | −2 | | | | |
| AMPK A1/B1/G1 | −1 | | | | |
| AURKA | −1 | >10 | 5.26 | 0.05 | >10 |
| BRAF | 18 | | | | |
| BTK | 4 | | | | |
| CAMK2D | 41 | 0.276 | 0.275 | 0.615 | 1.47 |
| CDK1/cyclinB | 0 | | | | |
| CDK5/p35 | 1 | >10 | >10 | 0.258 | >10 |
| CHEK1 | 3 | >10 | >10 | 0.999 | >10 |
| CK1 gamma2 | 6 | | | | |
| CK2 alpha 1 | 6 | | | | |
| DCK2 | 8 | | | | |
| DYRK3 | 2 | | | | |
| EGFR | −3 | | | | |
| EPHA2 | 2 | | | | |
| HER2 | 4 | | | | |
| IRAK4 | −1 | >10 | 3.44 | 0.655 | >10 |
| FGFR1 | 9 | | | | |
| FLT3 | 8 | | | | |
| mTOR | 12 | | | | |
| GSK3beta | 0 | | | | |
| IGF1R | 7 | | | | |
| IKKbeta | 7 | | | | |
| INSR | 4 | | | | |
| JAK3 | 2 | | | | |
| KDR | 1 | | | | |
| KIT | 4 | | | | |
| LCK | 3 | | | | |
| MARK2 | 5 | 7.94 | 1.84 | 0.434 | >10 |
| MEK1 | 16 | | | | |
| TAK1-TAB1 | −3 | | | | |
| MLK1 | −17 | | | | |
| HGK | −18 | | | | |
| ERK2 | 8 | | | | |
| P38 alpha | 12 | | | | |
| JNK1 | −5 | | | | |
| MAPKAPK2 | 4 | | | | |
| cMet | 7 | | | | |
| NEK1 | 10 | | | | |
| TRKA | 11 | | | | |
| PAK4 | 1 | | | | |
| PDGFRbeta | 4 | | | | |
| PHKG2 | 3 | | | | |
| PIM1 | −2 | | | | |
| PLK1 | 12 | | | | |
| PKA | 2 | | | | |
| PKC beta1 | 4 | | | | |
| RET | 3 | | | | |
| ROCK1 | 1 | | | | |
| RSK2 | 3 | | | | |
| P70S6K | 5 | | | | |
| SRC | 13 | | | | |
| SYK | 8 | | | | |
| Tie2 | −2 | | | | |

### Example 4

### In Vitro Neuroprotection Assay

Primary mouse cortical neurons are prepared from E15.5 embryos. Briefly, cortices are dissected, treated with trypsin and mechanically dissociated. Neurons are suspended in Neurobasal medium supplemented with B27, and plated on poly-D-lysine coated 48 well-plates. Neurons are grown in Neurobasal medium supplemented with B27 supplement and 0.2 mM Glutamine in 5% CO2 at 37° C.

At DIV 7, transient transfection of primary cortical cultures is performed with LipofectAMINE 2000 in OptiMEM as recommended by the manufacturer's protocol. Cells are cotransfected with 0.5 μg of a plasmid encoding full-length LRRK2 (WT or G2019S or other mutant) and 0.05 μg of a plasmid encoding GFP. After 5 h, the cultures are rinsed with fresh Neurobasal medium. Inhibitors are added in the fresh medium after transfection, and remain in the medium until the end of the experiment.

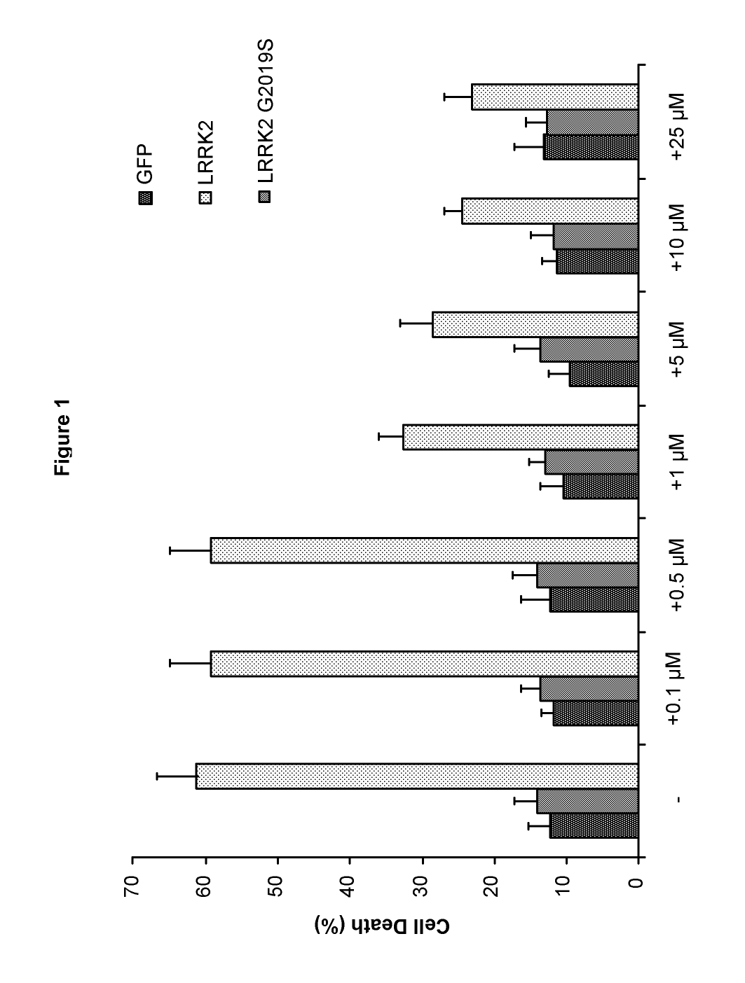

After 48 h, cells are fixed with 4% paraformaldehyde in PBS for 30 minutes. After 3 washes with PBS, cells are treated with 0.8 μg/mL of bisbenzimide (Hoechst). Cells are analyzed under a Zeiss inverted fluorescence microscope and images are digitized automatically from 50 independent fields per well using Axiovision acquisition software. Transfected cells are visualized by GFP, and quantification of cell viability using the Volocity software (Perkin Elmer) is achieved by automated measurement of the average intensity of DAPI stained nuclei of transfected cells. Cells with nuclei greater than a specified threshold for brightness (usually set at around 200%, based on positive control cells) and smaller than a specified threshold for size (usually set at around 50%, based on positive control cell) are counted by the software as non-viable. These measurements are correlated with other indices of cell death such as morphological measurements or capase 3/7 label. FIG. 1 shows that treatment with a compound of Formula I-A reduced intrinsic cell toxicity, i.e., the compound has a neuroprotective effect.

### Example 5

### Clinical Trial for Parkinson's Disease

### Purpose

This is a multi-center, double-blind, placebo controlled clinical trial to test two dosages of orally administered compound of Formula (I-A) for safety, tolerability, and/or futility.

The study will measure disease progression by the change in total UPDRS score between the baseline visit and 44 weeks. The compound will be administered daily once a day.

### Primary Outcome Measures:

Change in total Unified Parkinson's Disease Rating Scale (UPDRS) score from baseline to 44 weeks [Time Frame: 44 weeks]; Change in total UPDRS score from baseline to 44 weeks.

### Secondary Outcome Measures:

Change in Individual Parts I-IV of the Unified Parkinson's Disease Rating Scale (UPDRS) from baseline to 44 weeks [Time Frame: 44 weeks];

Compare the scores of the individual parts of the UPDRS. Part 1 assesses mentation, behavior and mood. Part II assesses activities of daily living in the week prior to the designated visit. Part III assesses the motor abilities at the time of the visit. Part IV assesses complications of therapy, for example (e.g.) dyskinesia, fluctuation, sleep disturbances, symptomatic orthostasis.

Change in Ambulatory Capacity from Baseline to 44 weeks [Time Frame: 44 weeks] This is the sum of the 5 UPDRS questions regarding ambulatory capacity: falling, freezing, walking, gait, postural stability.

Change in Schwab and England scale from baseline to 44 weeks [Time Frame: 44 weeks] The Schwab & England scale is an investigator and subject assessment of the subject's level of independence. The subject will be scored on a percentage scale reflective of his/her ability to perform acts of daily living in relation to what he′she did before Parkinson's disease appeared.

Change in Parkinson's Disease Questionnaire (PDQ-39) from baseline to 44 weeks [Time Frame: 44 weeks] The Parkinson's Disease Questionneire (PDQ-39) is a short, 39 item measure of quality of life in subjects with Parkinson's disease. The questionnaire covers 8 aspects of quality of life: mobility, activities of daily living, emotional well-being, stigma, social support, cognitions, communication and bodily discomfort.

Change in the Mattis Dementia Rating Scale (DRS-2) from baseline to 44 weeks [Time Frame: 44 weeks]. The Mattis dementia rating scale is a psychometric instrument designed to assess the extent and nature of dementia. The scale consists of content that covers: attention, initiation/preservation, construction, conceptualization, and memory.

Change in the 15-item Geriatric Depression Scale (GDS-15) from baseline to 44 weeks [Time Frame: 44 weeks]. The Geriatric Depression Scale—15 is a short 15 yes or no question instrument for assessing depression in the elderly. It has been found to be particularly useful in assessing depression in Parkinson's Disease.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A compound, or salt thereof, of formula (I-Z-2): 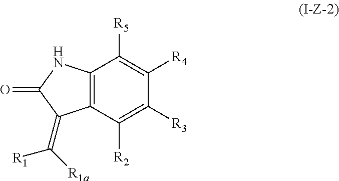
wherein
R₁ is pyrazole, imidazol-5-yl, triazolyl, triazolonyl, indoly-2-yl, indol-4-yl, indol-5-yl, indole-6-yl, indol-7-yl, benzimidazolyl, azabenzimidazolyl, azaindolyl, benzothiazolyl or benzoxazolyl, where R₁ is optionally substituted with one, two, three, four, or five R₆;
each R₆ is independently hydroxy, halo, optionally substituted alkyl, optionally substituted alkoxy, optionally substituted cycloalkyl, optionally substituted aryl, optionally substituted heteroaryl, optionally substituted heteroalkyl optionally substituted heteroalicyclyl, optionally substituted alkylcycloalkyl, optionally substituted alkylheteroalicyclyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₁ₐ is hydrogen, optionally substituted alkyl, optionally substituted heteroalkyl, optionally substituted cycloalkyl or optionally substituted heteroalicyclyl;
each of R₂ and R₄ is independently hydrogen, hydroxy, halo, haloalkyl, haloalkoxy, alkyl, cycloalkyl, alkoxy, aryl, heteroaryl, or heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₃ is hydroxy, halo, optionally substituted alkyl, optionally substituted cycloalkyl, optionally substituted alkoxy, optionally substituted cycloalkyloxy, optionally substituted aryl, optionally substituted heteroaryl, optionally substituted heteroalkyl, C(O)R, C(O)OR, NR′R″, NR′C(O)R″, NRC(O)NR′R″, NR′S(O)₂R″, C(O)NR′R″, S(O)₂R, or S(O)₂NR′R″;
R₅ is hydrogen, halo, haloalkyl or alkyl; and
each of R, R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

2. The compound of claim 1, or salt thereof, having the structure of formula (I-Z-2-a), formula (I-Z-2-b), formula (I-Z-2-c) or formula (I-Z-2-d):

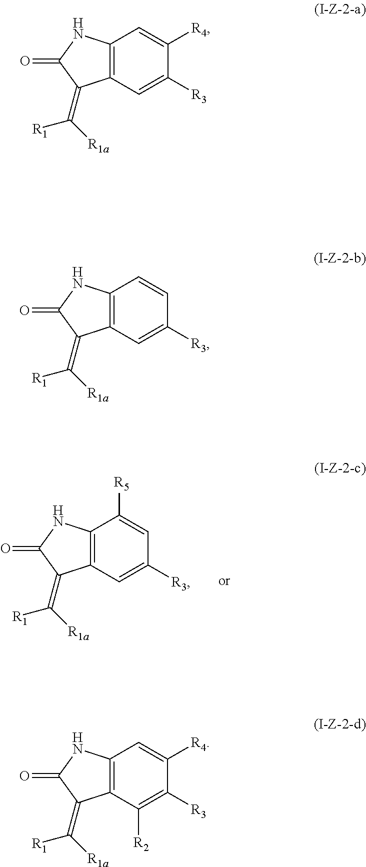

3. The compound of claim 1, or salt thereof, wherein R₁ₐ is hydrogen.

4. The compound of claim 1, or salt thereof, wherein R₁ₐ is C₁-C₃ alkyl.

5. The compound of claim 1, or salt thereof, wherein R₁ₐ is optionally substituted alkyl or optionally substituted heteroalkyl.

6. The compound of claim 1, or salt thereof, wherein R₁ₐ is optionally substituted cycloalkyl or optionally substituted heteroalicyclyl.

7. The compound of claim 1, or salt thereof, wherein R₁ is pyrazolyl, yl, triazolyl, triazolonyl, indoly-2-yl, indol-4-yl, indol-5-yl, indole-6-yl, indol-7-yl, benzimidazolyl, or azaindolyl where R₁ is optionally substituted with one or two R₆.

8. The compound of claim 1, or salt thereof, wherein R₁ is imidazol-5-yl and has a structure of: 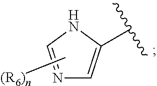
and
n is 0, 1, 2, or 3.

9. The compound of claim 8, or salt thereof, wherein R₁ is imidazol-5-yl, optionally substituted with one R₆.

10. The compound of claim 1, wherein R₆ is independently hydroxy, halo, optionally substituted C₁-C₆alkyl, optionally substituted C₂-C₆ heteroalkyl, optionally substituted C₃-C₇cycloalkyl, optionally substituted C₁-C₆alkoxy, optionally substituted aryl, optionally substituted heteroaryl, optionally substituted C₃-C₇heteroalicyclyl, or NR′R″, where alkyl, alkoxy, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₆alkoxy, haloC₁-C₆alkoxy, amino(C₁-C₆alkyl), amino(C₁-C₆alkyl)₂, C₂-C₆heteroalkyl, SO₂(C₁-C₃alkyl), NR′C(O)R″, C(O)NR′R″, C₃-C₆cycloalkyl, and C₃-C₇heteroaliyclyl; and where R′ and R″ are independently hydrogen, C₁-C₆alkyl, haloC₁-C₆alkyl, C₃-C₇cycloalkyl, aryl, heteroaryl, or C₁-C₆heteroalkyl.

11. The compound of claim 1, wherein R₆ is independently hydroxy, F, Cl, Br, I, CH₂F, CHF₂, CF₃, CH₂CH₂F, CH₂CHF₂, CH₂CF₃, methyl, ethyl, propyl, butyl, pentyl, hexyl, iso-propyl, cyclopropyl, cyclobutyl, cyclopentyl, cyclohexyl, methoxy, ethoxy, propoxy, iso-propoxy, phenyl, pyridyl, OCH₂F, OCHF₂, OCF₃, OC(═O)Me, CO₂Me, CO₂Et, CO₂H, NHC(O)Me, C(O)NMe₂, C(O)NH₂, C(O)NHMe, SO₂Me, SO₂Et, or SO₂NMe₂.

12. The compound of claim 1, wherein R₆ is independently optionally substituted C₁-C₆alkyl, optionally substituted C₂-C₆heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl.

13. The compound of claim 1, wherein R₆ is methyl, ethyl, propyl, isopropyl, butyl, isobutyl, tert-butyl, pentyl, neo-pentyl, or isobutyl; or R₆ is tetrahydropyranyl, piperidinyl, piperazinyl, morpholinyl, pyrrolidinyl, pyrrolyl, phenyl, pyridinyl, or pyrimidinyl, where tetrahydropyranyl, piperidinyl, piperazinyl, morpholinyl, pyrrolidinyl, pyrrolyl, phenyl, pyridinyl, or pyrimidinyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl.

14. The compound of claim 1, wherein R₂ is hydrogen, hydroxy, F, Cl, CH₂F, CHF₂, CF₃, CH₂CH₂F, CH₂CHF₂, CH₂CF₃, methyl, ethyl, propyl, iso-propyl, cyclopropyl, cyclobutyl, cyclopentyl, methoxy, ethoxy, iso-propoxy, OCF₃, C(O)NMe₂, or SO₂Me.

15. The compound of claim 1, wherein R₂ is hydrogen.

16. The compound of claim 1, wherein R₃ is halo, haloalkyl, haloalkoxy, alkyl, or alkoxy.

17. The compound of claim 1, wherein
R₃ is hydroxy, halo, halo C₁-C₆alkyl, halo C₁-C₆alkoxy, C₁-C₆alkyl, hetero C₁-C₆alkyl, C₃-C₆cycloalkyl, aryl, heteroaryl, C₁-C₆alkoxy, NR′R″, NR′C(O)R″, NRC(O)NR′R″, NR′S(O)₂R″, C(O)NR′R″, S(O)₂R, or S(O)₂NR′R″;
each of R′ and R″ are independently hydrogen, alkyl, haloalkyl, cycloalkyl, aryl, heteroaryl, or heteroalkyl; or
R′ and R″ taken together with the nitrogen to which they are attached form a ring structure that optionally includes an additional heteroatom selected from N or O and is optionally substituted.

18. The compound of claim 1, wherein R₃ is hydroxy, CH₂F, CHF₂, CF₃, CH₂CH₂F, CH₂CHF₂, CH₂CF₃, methyl, ethyl, methoxy, ethoxy, propoxy, iso-propyl, iso-propoxy, cyclopropyl, cyclobutyl, cyclopentyl, OCF₃, C(O)NMe₂, or SO₂Me.

19. The compound of claim 1, wherein R₃ is methoxy, ethoxy, propoxy, or iso-propoxy.

20. The compound of claim 1, wherein R₄ is hydrogen, hydroxy, halo, haloC₁-C₆alkyl, C₁-C₆alkyl or C₁-C₆alkoxy.

21. The compound of claim 1, wherein R₄ is hydrogen, hydroxy, F, Cl, Br, I, CH₂F, CHF₂, CF₃, CH₂CH₂F, CH₂CHF₂, CH₂CF₃, methyl, methoxy, ethyl, ethoxy, propyl, propoxy, iso-propyl or isopropoxy.

22. The compound of claim 1, wherein R₄ is hydrogen or fluoro.

23. The compound of claim 1, wherein R₄ is C₁-C₃ alkoxy.

24. The compound of claim 1, wherein R₅ is hydrogen, halo, alkyl or haloalkyl.

25. The compound of claim 1, wherein R₅ is hydrogen or halo.

26. The compound of claim 1, wherein R₅ is hydrogen or fluoro.

27. The compound of claim 1, wherein
R₁ is imidazol-5-yl optionally substituted with one or two R₆;
R₆ is independently optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl; and
R₃ is hydroxy, halo, haloalkyl, haloalkoxy, alkyl, or alkoxy.

28. The compound of claim 1, wherein
R₁ is imidazol-5-yl optionally substituted with one or two R₆;
R₆ is independently optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl; and
R₄ is hydrogen, halo, haloalkyl, haloalkoxy, alkyl, or alkoxy.

29. The compound of claim 1, wherein
R₁ is imidazol-5-yl optionally substituted with one or two R₆;
R₆ is independently optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl; and
R₃ is hydroxy, halo, haloalkyl, haloalkoxy, alkyl, or alkoxy;
R₄ is hydrogen, hydroxy, halo, haloalkyl, haloalkoxy, alkyl, or alkoxy; and
R₂ and R₅ are hydrogen.

30. The compound of claim 1, wherein
R₁ is imidazol-5-yl optionally substituted with one or two R₆;
R₆ is independently optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, optionally substituted heteroaryl, or NR′R″, where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂(C₁-C₃alkyl), C₃-C₆cycloalkyl, and C₃-C₇heteroaliyclyl; and R′ and R″ are independently selected from hydrogen and C₁-C₆ alkyl; and
R₃ is hydroxy, halo, haloalkyl, haloalkoxy, alkyl, or alkoxy;
R₅ is hydrogen, or halo; and
R₂ and R₄ are hydrogen.

31. A compound, or salt thereof, of Formula (I-Z-3-a): 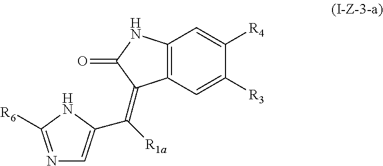
wherein
R₁ₐ is hydrogen, optionally substituted C₁-C₆ alkyl, optionally substituted C₃-C₇ cycloalkyl or optionally substituted C₃-C₇ heteroalicyclyl;
R₆ is optionally substituted C₁-C₆alkyl, optionally substituted C₂-C₆ heteroalkyl, optionally substituted C₃-C₇cycloalkyl, optionally substituted C₃-C₇heteroalicyclyl, optionally substituted aryl, or optionally substituted heteroaryl;
R₃ is halo, alkoxy or haloalkoxy;
R₄ is hydrogen or halo;
where alkyl, heteroalkyl, cycloalkyl, aryl, heteroaryl and heteroalicyclyl are optionally substituted with one or two groups selected from oxo, hydroxy, amino, cyano, halo, C₁-C₆alkyl, haloC₁-C₆alkyl, C₁-C₆alkoxy, haloC₁-C₆alkoxy, amino(C₁-C₆alkyl), amino(C₁-C₆alkyl)₂, C₂-C₆heteroalkyl, SO₂ (C₁-C₃alkyl), C₃-C₆cycloalkyl, and C₃-C₇heteroaliyclyl.

32. A compound selected from
(3Z)-3-[(3-methyl-1H-pyrazol-5-yl)methylene]indolin-2-one
(3Z)-5-methoxy-3-[(2-methyl-1H-imidazol-5-yl)methylene]indolin-2-one
(3Z)-3-(1H-pyrrolo[2,3-b]pyridin-2-ylmethylene)indolin-2-one
(3E)-3-(1,3-benzoxazol-2-ylmethylene)indolin-2-one
(3Z)-6-methoxy-3-[(2-methyl-1H-imidazol-5-yl)methylene]indolin-2-one
(3E)-3-(1,3-benzothiazol-2-ylmethylene)indolin-2-one
(3E)-3-(3-quinolylmethylene)indolin-2-one
(3Z)-5-chloro-3-[(2-methyl-1H-imidazol-5-yl)methylene]indolin-2-one
(3Z)-3-[(2-methyl-1H-imidazol-5-yl)methylene]-5-(trifluoromethoxy)indolin-2-one
(3Z)-3-[(2-isopropyl-1H-imidazol-5-yl)methylene]-5-methoxy-indolin-2-one
(3Z)-5-ethoxy-3-[(2-methyl-1H-imidazol-5-yl)methylene]indolin-2-one
(3Z)-3-[(2-methyl-1H-imidazol-5-yl)methylene]-5-methylsulfonyl-indolin-2-one
(3Z)-3-[(2-ethyl-1H-imidazol-5-yl)methylene]-5-methoxy-indolin-2-one
(3Z)-5-methoxy-3-[[2-(methoxymethyl)-1H-imidazol-5-yl]methylene]indolin-2-one
(3Z)-3-[(2-tert-butyl-1H-imidazol-5-yl)methylene]-5-methoxy-indolin-2-one
(3Z)-3-[[2-(2,6-difluorophenyl)-1H-imidazol-5-yl]methylene]-5-methoxy-indolin-2-one
(3Z)-5-isopropoxy-3-[(2-isopropyl-1H-imidazol-5-yl)methylene]indolin-2-one
(3Z)-3-[(2-isobutyl-1H-imidazol-5-yl)methylene]-5-isopropoxy-indolin-2-one
(3Z)-5-isopropoxy-3-[[2-(morpholinomethyl)-1H-imidazol-5-yl]methylene]indolin-2-one
(3Z)-5-methoxy-3-[[2-(2-methoxyethyl)-1H-imidazol-5-yl]methylene]indolin-2-one
(3Z)-5-isopropoxy-3-[(5-isopropyl-4H-1,2,4-triazol-3-yl)methylene]indolin-2-one
(3Z)-5-isopropoxy-3-[(2-morpholino-1H-imidazol-5-yl)methylene]indolin-2-one
(3Z)-5-hydroxy-3-[(2-tetrahydropyran-4-yl-1H-imidazol-5-yl)methylene]indolin-2-one
(3Z)-5-ethyl-3-[(2-tetrahydropyran-4-yl-1H-imidazol-5-yl)methylene]indolin-2-one
(3Z)-5-isopropoxy-3-[(2-tetrahydropyran-4-yl-1H-imidazol-5-yl)methylene]indolin-2-one
(3Z)-3-[[2-[1-(2-fluoroethyl)-4-piperidyl]-1H-imidazol-5-yl]methylene]-5-isopropoxy-indolin-2-one
(3Z)-5-chloro-3-[(2-tetrahydropyran-4-yl-1H-imidazol-5-yl)methylene]indolin-2-one
(3Z)-5-chloro-3-[[2-[1-(2-fluoroethyl)-4-piperidyl]-1H-imidazol-5-yl]methylene]indolin-2-one
(3Z)-5-(2-fluoroethoxy)-3-[(2-tetrahydropyran-4-yl-1H-imidazol-5-yl)methylene]indolin-2-one
(3Z)-3-[[2-(dimethylamino)-1H-imidazol-5-yl]methylene]-5-isopropoxy-indolin-2-one
(3Z)-7-fluoro-5-isopropoxy-3-[(2-morpholino-1H-imidazol-5-yl)methylene]indolin-2-one
(3Z)-6-fluoro-5-isopropoxy-3-[(2-morpholino-1H-imidazol-5-yl)methylene]indolin-2-one
(3Z)-4-fluoro-5-isopropoxy-3-[(2-morpholino-1H-imidazol-5-yl)methylene]indolin-2-one
(3Z)-5-isopropoxy-3-[1-(2-morpholino-1H-imidazol-5-yl)ethylidene]indolin-2-one
(3Z)-6-fluoro-5-isopropoxy-3-[(2-tetrahydropyran-4-yl-1H-imidazol-5-yl)methylene]indolin-2-one
(3Z)-6-fluoro-5-isopropoxy-3-[1-(2-morpholino-1H-imidazol-5-yl)ethylidene]indolin-2-one
(3Z)-6-fluoro-3-[[2-[1-(2-fluoroethyl)-4-piperidyl]-1H-imidazol-5-yl]methylene]-5-isopropoxy-indolin-2-one and
(3Z)-3-[[2-(4-ethylpiperazin-1-yl)-1H-imidazol-5-yl]methylene]-6-fluoro-5-isopropoxy-indolin-2-one.

33. A pharmaceutical composition comprising a compound of claim 1, and a pharmaceutically acceptable carrier, excipient, or binder.

34. A method of treating an individual suffering from or susceptible to a neurodegenerative disease comprising administration of a compound of claim 1 to the individual in need thereof.

35. A method for inhibiting a leucine-rich repeat kinase-2 (LRRK2) kinase, the method comprising contacting an LRRK2 kinase with a compound of claim 1.

36. A method for treating a disorder or condition that is treated by inhibiting LRRK2 activity in a subject in need of treatment thereof, the method comprising administering to the subject a therapeutically effective amount of a compound of claim 1.

37. The method of claim 34, wherein the compound is radiolabeled.
